SEQUENCE LISTING (5 sequences)

SEQ ID NO: 1 - protein, 14 aa, synthetic construct
GYINPRNDYTEYNQ

SEQ ID NO: 2 - protein, 14 aa, synthetic construct
GYINPRNDYTEYNQ

SEQ ID NO: 3 - protein, 6 aa, synthetic construct
RNDYTE

SEQ ID NO: 4 - protein, 21 aa, synthetic construct
  DISULFID 1..21: note = a disulfide bond between the N- and C-terminal cysteines
CLGIIWGDGRTDYNSALKSRC

SEQ ID NO: 5 - protein, 69 aa, synthetic construct
RALLSIFGSLDHRHHHRTCNTTHYRVTTMSHPQFEKKKKLRMKMSHPQLINTTHYRGGPTMGGSPSRQV

(19) United States
(12) Patent Application Publication
(10) Pub. No.: US 2024/0216490 A1
(43) Pub. Date: Jul. 4, 2024

(54) Autoantigens Engineered to Suppress Autoimmune Response

(71) Applicant: Osprey Biopharmaceuticals, Inc., San Diego, CA (US)

(72) Inventors: Richard James Glynne, San Diego, CA (US); Tigran Aivazian, San Diego, CA (US); James Paulson, San Diego, CA (US)

(21) Appl. No.: 18/351,367

(22) Filed: Jul. 12, 2023

Related U.S. Application Data
(60) Provisional application No. 63/388,887, filed on Jul. 13, 2022.

(51) Int. Cl. A61K 39/00; A61P 37/06

(52) U.S. Cl. CPC A61K 39/001113; A61P 37/06; A61K 2039/545

(57) ABSTRACT

The present disclosure provides engineered antigen compositions that reduce immune response to the antigen in a subject having an immune response to the antigen. The present disclosure also provides pharmaceutical compositions that include such engineered autoantigens and methods for making such engineered autoantigens. Methods for using such engineered antigens as therapeutics and in research are also disclosed.

### CROSS REFERENCE TO APPLICATIONS

This application claims priority to U.S. Provisional Application 63/388,887, filed Jul. 13, 2022, which is incorporated herein by reference.

### INCORPORATION BY REFERENCE OF SEQUENCE LISTING PROVIDED AS AN XML FILE

A Sequence Listing is provided herewith as a Sequence Listing XML, “NHWK-002_SEQ_LIST” created on Jul. 12, 2023 and having a size of 5,915 bytes. The contents of the Sequence Listing XML are incorporated by reference herein in their entirety.

### FIELD OF THE INVENTION

This invention pertains to autoantigens that have been engineered to suppress an immune response to the autoantigens in patients.

### BACKGROUND OF THE INVENTION

Autoimmune disorders are caused when the immune system destroys native tissues, cells, or biomolecules by, e.g., mounting an immune response to self-antigens. Immune disorders are often treated with systemic immune suppression, e.g., by administering antibodies, steroids, gene therapy, etc.. Such treatments are not always target specific and/or effective, resulting in compromised immune responses and side effects. Such treatments, when effective, may only be effective transiently.

### SUMMARY OF THE INVENTION

Engineered autoantigens that suppress or eliminate an immune response to the native autoantigen in an individual are provided. The present disclosure also provides pharmaceutical compositions that include such engineered autoantigens and methods for making such engineered autoantigens. Methods for using engineered antigens as therapeutics and in research are also disclosed.

### BRIEF DESCRIPTION OF THE DRAWINGS

FIG. 1 depicts an aspect of the model for CD22-engaging autoantigens that suppress, anergize, and/or delete pathogenic, autoantigen-specific B cells: B cell receptor—Siglec-2 co-engagers (including Autoantigen-Siglec Ligand conjugates and Autoantigen-CD22 binder fusions) suppress, silence, and/or delete autoantigen-specific, pathogenic B cells by virtue of the physical recruitment of the inhibitory CD22 receptor to the B cell receptor complex.

FIG. 2 depicts another aspect of the model for CD22-engaging autoantigens that suppress, anergize, and/or delete pathogenic, autoantigen-specific B cells: B cell receptor—Siglec-2 co-engagers (including Autoantigen-Siglec Ligand conjugates and Autoantigen-CD22 binder fusions) act only on autoantigen-specific B cells (suppression, silencing, and/or deletion) without exerting such effects on non-autoantigen-specific B cells and thus leaving the non-autoantigen-specific portion of the B cell repertoire intact.

FIG. 3 depicts multiple formats for Siglec-B cell receptor-coengaging biologics, based on disease-driving autoantigens, that tolerize pathogenic B cells that cause an autoimmune disease: Format 1. Siglec Ligand-Modified Autoantigen: Non-Enzymatic or enzymatic conjugation. Site-specific or non-site specific. Format 2. Protein/Peptide Fusion: CD22-Binding Domains or Peptides are incorporated into the autoantigen through in-frame fusion or conjugation. Format 3: Siglec Ligand-Modified Glycan (in-cell or in vitro): Biosynthetic, enzymatic glycan modification (e.g., during protein expression) or in vitro Enzymatic Glycan Modification post-expression.

FIG. 4 depicts the results of an experiment demonstrating the induction of tolerance in naïve animals to adalimumab and the prevention of anti-adalimumab antibody generation following adalimumab challenge. Prophylactic tolerization of mice to adalimumab was achieved through the pre-administration of one or two doses of adalimumab-CD22 ligand conjugates. Anti-adalimumab antibody responses were measured after 4 subcutaneous doses with 4 mg/kg adalimumab, evaluating the effects of adalimumab-SigL or vehicle predosing on subsequent responses to adalimumab antigen. See panel A. “Adal” is adalimumab. “SigL-Adal” is adalimumab-CD22 ligand conjugate. Adalimumab-CD22 ligand conjugate bears BPC-Neu5Gc-based Siglec Ligand-Linker structures. Serum IgG levels against adalimumab/adalimumab conjugate were measured 28 days after the first adalimumab dose. FIG. 4, panel B shows individual animal serum IgG titer values against adalimumab and/or adalimumab-SigL conjugate (n=5 mice).

FIG. 5 depicts the results of an experiment demonstrating the lack of anti-drug antibody induction from adalimumab-SigL dosing in animals that had been pre-dosed with unmodified adalimumab. To evaluate the dependence of anti-drug antibody responses on the number of adalimumab doses, unmodified adalimumab was dosed subcutaneously (4 mg/kg) from 1 to 4 times, each dose 1 week apart. Panel A shows the dosing scheme for 5 different groups (A to E). Panel B shows individual animal serum IgG titer values (n=5 mice) against adalimumab and/or adalimumab-SigL conjugate. “Adal” is adalimumab. “SigL-Adal” is adalimumab-CD22 ligand conjugate. Adalimumab-CD22 ligand conjugate bears BPC-Neu5Gc-based Siglec Ligand-Linker structures. Serum IgG levels against adalimumab/adalimumab conjugate were measured 27 days after the first adalimumab dose.

FIG. 6 depicts an aspect of the model for autoantigen-CD22 binder fusion proteins that suppress, anergize, and/or delete pathogenic, autoantigen-specific B cells: B cell receptor—Siglec-2 binder fusion proteins suppress, silence, and/or delete autoantigen-specific, pathogenic B cells by virtue of the physical recruitment of the inhibitory CD22 receptor to the B cell receptor complex.

FIG. 7 depicts another aspect of the model for autoantigen-CD22 binder fusion proteins that suppress, anergize, and/or delete pathogenic, autoantigen-specific B cells: B cell receptor—Siglec-2 binder fusion proteins act only on autoantigen-specific B cells (suppression, silencing, and/or deletion) without exerting such effects on non-autoantigen-specific B cells and thus leaving the non-autoantigen-specific portion of the B cell repertoire intact.

FIG. 8, panels A and B, depict an in vitro B cell activation assay where mouse primary B cells are treated with beads coated with different combinations of B cell receptor agonizing anti-IgD antibody, anti-CD22 antibody, and/or isotype controls. The treatment groups in FIG. 8 are: 1) beads alone, 2) isotype antibody-coated beads, 3) anti-IgD beads, 4) beads coated with both anti-IgD and anti-CD22, 5) anti-CD22 beads, and 6) pooled beads coated separately with anti-IgD and anti-CD22. Splenocytes from C57BL6 mice were incubated with coated beads and compared for activation, as measured by CD69 upregulation. CD69 levels on the different treatment groups are evaluated through the percentage of cells that are CD69-positive (FIG. 8, panel B).

FIG. 9 depicts an in vitro B cell activation assay, using human primary B cells and either a B cell receptor agonizing anti-IgM antibody or an anti-IgM x anti-CD22 fusion bispecific. The domain structures of the two test articles are shown in FIG. 9, panel A. The B cell stimulatory activities of anti-IgM and anti-IgM-Siglec Ligand conjugates are compared in dose titration experiments with an activation readout of CD69 upregulation (FIG. 9, panel B). CD69 levels on the different treatment groups are evaluated through the percentage of cells that are CD69-positive.

FIG. 10 depicts the evaluation of anti-IgM x anti-CD22 fusion bispecific binding to human primary B cells, in comparison with the parental, unconjugated anti-IgM antibody. Binding is evaluated by fluorescence cytometry, in a competition assay format with Alexa-647-labeled anti-IgM antibody. FIG. 10, panel A depicts dose-response results for concentration-dependent inhibition of fluorescently-labeled anti-IgM binding to IgM+ B cells by unlabeled anti-IgM x anti-CD22 fusion bispecifics and parental, unlabeled anti-IgM antibody. Parental anti-IgM and anti-IgM x anti-CD22 fusion bispecific show similar inhibition of labeled anti-IgM and thus are similar in activity for surface IgM binding on B cells. FIG. 10, panel B is a schematic for the binding assay system.

FIG. 11 depicts the results of a continuation of the study depicted in FIG. 4 with further measurement of serum anti-adalimumab or adalimumab-Siglec Ligand titers. In this depiction, mouse anti-adalimumab titers were further monitored out to time points of 100-110 days post-first dose. As described in FIG. 4, prophylactic tolerization of mice to adalimumab was achieved through the pre-administration of one (FIG. 11, panel A) or two (FIG. 11, panel B) doses of adalimumab-CD22 ligand conjugates. Anti-adalimumab antibody responses were measured after 4 subcutaneous doses with 4 mg/kg adalimumab, evaluating the effects of 4 mg/kg adalimumab-SigL or vehicle pre-dosing on subsequent responses to adalimumab antigen. “Adal” is adalimumab. “SigL-Adal” is adalimumab-CD22 ligand conjugate. Adalimumab-CD22 ligand conjugate bears BPC-Neu5Gc-based Siglec Ligand-Linker structures.

FIG. 12 depicts tolerization of mice to subsequent dosing with immunogenic adalimumab antibody. Mice were administered either 4 weekly doses of 4 mg/kg adalimumab-Siglec Ligand LDR 9.0 conjugate (solid circles) or 4 mg/kg adalimumab (open circles), followed by 4 doses of 4 mg/kg adalimumab at days 91, 147, 174, and 202. Serum was sampled for anti-drug antibody analysis at days 0, 28, 112, 146, 168, 201, and 232. As in the experiments depicted in FIG. 4 and FIG. 11, mice pre-administered with adalimumab-Siglec Ligand show strong suppression of anti-drug antibody. In addition, mice dosed with adalimumab at much later time points continue to show resistance to adalimumab immunogenicity, demonstrating strong and durable tolerization induced by the Siglec Ligand conjugate. The mice are specifically tolerized to subsequent dose with adalimumab immunogen even as late as day 202.

FIG. 13 depicts the cytometry analysis of splenic, antigen-specific B cells from mice dosed subcutaneously with either vehicle, 4 mg/kg adalimumab, or 4 mg/kg adalimumab-Siglec Ligand LDR 6.4. Adalimumab-specific B cells (CD45⁺CD19⁺IgD⁻Adal⁺) were evaluated by flow cytometry 7 days post adalimumab or SigL-adalimumab challenge. Antigen-specific B cells were detected in the spleens in all mice dosed with adalimumab but absent in the spleens of mice dosed with adalimumab-Siglec Ligand.

FIG. 14 depicts cytometry analysis of splenic antigen-specific B cells and germinal center B cells after dosing with sequences of adalimumab, adalimumab-Siglec Ligand, and/or drug vehicle. Mice were dosed subcutaneously on either day 0 or day 7 with vehicle (PBS), 4 mg/kg adalimumab, or 4 mg/kg adalimumab-Siglec Ligand LDR 8.4. Dosing sequences for the different animal groups are shown in FIG. 14, panel A. Adalimumab-specific B cells (CD45⁺CD19⁺IgD⁻Adal⁺) were evaluated by flow cytometry at day 14 (FIG. 14, panel B). Germinal center B cells (CD19⁺CD95⁺GL7⁺) were harvested on day 14 after adalimumab, adalimumab-Siglec Ligand, or vehicle challenges on days 0 and 7 (FIG. 14, panel C).

FIG. 15 depicts cytometric analysis of splenic antigen-specific B cells after dosing with sequences of adalimumab, adalimumab-Siglec Ligand, and/or drug vehicle. Mice were dosed subcutaneously on days 0 and 3 with either vehicle (PBS), 4 mg/kg adalimumab, or 4 mg/kg adalimumab-Siglec Ligand LDR 9.2 as shown in FIG. 15, panel A. Adalimumab-specific B cells (CD45⁺CD19⁺IgD⁻Adal⁺) were evaluated by flow cytometry at day 7 (FIG. 15, panel B). Dosing of mice with adalimumab-Siglec Ligand 3 days after adalimumab dosing was sufficient to block the differentiation and expansion of adalimumab-specific B cells.

FIG. 16 depicts an experiment evaluating the immunogenicity in mice of adalimumab versus engineered adalimumab x anti-mCD22 scFv bispecific. FIG. 16, panel A is a schematic for the bispecific adalimumab-anti-mouseCD22 scFv fusion protein and the parental adalimumab monoclonal antibody. FIG. 16, panel B depicts anti-drug antibody responses in mice for adalimumab and adalimumab-anti-mCD22-scFv fusion protein at day 20. FIG. 16, panel C depicts anti-drug antibody responses in mice for adalimumab and adalimumab-anti-mCD22-scFv fusion protein at day 27. Mice received a single 4 mg/kg subcutaneous dose of adalimumab or adalimumab-anti-mCD22-scFv fusion protein. Serum IgG levels against adalimumab/adalimumab conjugates were measured at days 20 (FIG. 16, panel B) and 27 (FIG. 16, panel C). FIG. 16, panel B and FIG. 16, panel C show individual and median animal serum IgG titer values (n=5 mice).

### DETAILED DESCRIPTION OF THE INVENTION

Engineered autoantigens (as referred to as engineered AutoAg) are provided that suppress autoimmune response to the AutoAg in an individual. These engineered AutoAg are generated by engineering the AutoAg to comprise a modified Siglec ligand or binder profile relative to a corresponding non-engineered AutoAg. The Siglec ligand profile may be modified by conjugating to the AutoAg a Siglec ligand (SigL). The SigL may be a molecule comprising sialic acid or a Siglec (e.g., CD22)-binding polypeptide. An engineered AutoAg as disclosed herein may be used for treating a subject that has an autoimmune disease where the subject produces antibodies that bind to the AutoAg. The engineered AutoAg binds AutoAg-specific B-cell receptors on AutoAg-specific B cells and not to B cells that are not specific for the AutoAg. Due to the presence of the SigL in the same molecule, the AutoAg-SigL recruits CD22 to the AutoAg-specific B cell receptor triggering an AutoAg B cell receptor-CD22 clustering event. The triggered AutoAg B cell receptor-CD22 clustering event prevents activation of the B cell, resulting in prevention of differentiation of the B cell into a plasma cell that secretes antibodies that bind to the autoantigen. In addition, AutoAg B cell receptor-CD22 clustering also drives dominant, suppressive signals that trigger anergic, apoptotic, and/or other deletion mechanisms that result in suppression or elimination of autoimmune disease-driving, AutoAg-specific B cell clones.

Also provided herein is a method for reducing an immune response to an antigen in a subject having the immune response. The method may include administering an engineered antigen to the subject, where the engineered antigen comprises a modified Siglec ligand (SigL) profile relative to a corresponding non-engineered antigen. The SigL may be sialic acid or a polypeptide that binds to a siglec. The siglec may be present on a B cell and the engineered antigen may simultaneously bind to the siglec and to a BCR specific for the antigen portion of the engineered antigen.

These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a ligand” includes a plurality of such ligands and reference to “the peptide” includes reference to one or more peptides and equivalents thereof, e.g., polypeptides, known to those skilled in the art, and so forth.

The transitional term “comprising” is inclusive or open-ended and does not exclude additional, unrecited elements or method steps. The transitional phrase “consisting of” excludes any element, step, or ingredient that is not specified. The transitional phrase “consisting essentially of” defines the scope to the specified elements, materials or steps and those that do not materially affect the basic and novel characteristics of the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Autoantigens Engineered to Suppress Autoimmune Response

Disclosed herein are autoantigens engineered to suppress and/or eliminate immune responses to disease-driving autoantigens, methods for making such engineered autoantigens, and methods for use of such an autoantigen to suppress immune response to the autoantigen.

As used herein, an “autoantigen” (abbreviated as “AutoAg”) refers to an endogenous molecule present in a subject which the subject's immune system does not recognize as an endogenous molecule (i.e., as a self-antigen) and thus mounts an immune response to the self-antigen. Such an immune response is referred to as an autoimmune response. The autoantigen is encoded by an endogenous gene present in a subject and may be a polypeptide, e.g., a soluble or membrane-localized polypeptide, a lipidated, glycosylated, or otherwise post-translationally modified polypeptide; a nucleic acid (e.g., DNA, RNA); or a complex thereof. In some embodiments, the autoantigen is any autoantigen that elicits a B-cell driven immune response in an individual, where the individual produces B cells that bind to the autoantigen via B-cell receptors (BCRs) and upon binding to the autoantigen differentiate into plasma cells that produce autoantibodies that bind to the autoantigen.

In contrast to a biotherapeutic, an autoantigen is a molecule that is endogenously produced by the subject, whereas a biotherapeutic is a molecule that the subject does not produce and is exogenously supplied to the person, e.g., as a polypeptide or a gene encoding the polypeptide. Thus, an immune response to a biotherapeutic is not considered an autoimmune response. Rather, an immune response to a biotherapeutic is a normal immune response. A biotherapeutic may be composed of sugars, amino acids, proteins, lipids or nucleic acids or complex combinations of these substances. Unlike an engineered autoantigen, a biotherapeutic is not intended as an autoimmune-suppressive mimic of a non-suppressive and endogenous disease-driving antigen. Nonlimiting examples of biotherapeutics include protein therapeutics, e.g., antibody therapeutics, fusion protein therapeutics, enzyme therapeutics, viral therapeutics, cell therapeutics, and nucleic acid therapeutics.

Specific examples of biotherapeutics include a monoclonal antibody, a bispecific antibody, an scFv, a Fab, a camelid, or a nanobody, e.g., adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, or tocilizumab. In certain embodiments, the biotherapeutic is erythropoietin, thrombopoietin, human growth hormone, tissue factor, IFNβ-1b, IFNβ3-1a, IL-2 or the IL-2 mimetic aldesleukin, exenatide, albiglutide, alefacept, palifermin, or belatacept.

In certain embodiments, the biotherapeutic is an enzyme, such as, asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), Tripeptidyl peptidase (TPP1), Pseudomonas elastase (PaE), Pseudomonas alkaline protease (PaAP), or Streptococcal pyrogenic exotoxin B (SpeB). In certain embodiments, the biotherapeutic is not Factor VIII.

In some embodiments, an autoantigen, as provided herein, is a naturally occurring antigen in a healthy individual. Using PANTHER, (Protein ANalysis THrough Evolutionary Relationships), autoantigens can be classified into the following varieties based on function played by the antigen in-vivo that may have a role to play as, for example, an enzyme, intercellular adhesive protein, cell junction protein, cytoskeletal protein, extracellular matrix protein, cellular receptor, transcription or translational protein, gene editing protein, structural protein, or the like.

Autoantigens have been found to be associated with several autoimmune disorders. A comprehensive list of disorders and associated autoantigens can be found at the database AAgAtlas 1.0 database accessed by typing into a web browser http followed by://biokb.ncpsb.org/followed by aagatlas/). Nonlimiting examples of autoantigens and their associated disorders include: p53-associated autoimmune diseases lupus and scleroderma; Desmoglein (Dsg) 1 and 3 autoantigens associated with Pemphigus vulgaris; autoantigens Gliadin and type 2 transglutaminase associated with Celiac disease; autoantigens PDC-E2 (Pyruvate dehydrogenase complex component E2) and BCOADC-E2 (Branched chain 2-oxo-acid dehydrogenase complex component E2) associated with Primary Biliary Cholangitis; autoantigens PLA2R (Phospholipase A2 Receptor) and THSD7A (thrombospondin type-1 domain containing protein 7A) associated with Membranous Nephropathy; autoantigen TSHR (Thyroid-Stimulating Hormone Receptor) associated with Graves' Disease; autoantigens AChR (Acetylcholine Receptor), MuSK (Muscle-Specific Kinase), and LRP4 (associated with Myasthenia Gravis; and autoantigens associated with rheumatoid arthritis (RA) (e.g., citrullinated peptides and proteins, carbamylated proteins, acetylated proteins) and systemic lupus erythematosus (SLE) (e.g., anti-nuclear antibodies, anti-dsDNA antibodies, anti-nucleosome antibodies, and others), and the like.

In certain aspects, an engineered autoantigen that suppresses an ongoing autoimmune response to the autoantigen in a subject includes a siglec ligand. The engineered autoantigen is configured to both bind to BCRs on a B cell that recognizes the autoantigen and to bind to a siglec present on the B cell. The autoantigen portion of the engineered autoantigen provides specificity for targeting only B cells that bind to the autoantigen while the siglec ligand, while not specific to a particular B cell clone, may bind to any B cell expressing the siglec, prevents activation of the B cell. In some instances, the siglec ligand provides a therapeutic benefit to the individual by suppressing the individual's immune response to the autoantigen, where the immune response is reduced by 50% or more when the engineered autoantigen is administered to an individual relative to when the individual is administered the non-engineered version of the autoantigen. Further, in some instances, the immune reaction is reduced by 60%, 70%, 80% or more, for example 85%, 90%, 95% or more, in certain cases 98%, 99%, or 100%, i.e., such that the immune response is undetectable, i.e., the autoantigen is rendered nonimmunogenic.

In some aspects of the disclosure, an engineered autoantigen is provided, wherein the engineered autoantigen (referred to interchangeably as the “clonally immunosuppressive autoantigen”, “hypoimmunogenic autoantigen”, “modified autoantigen” or simply “subject autoantigen”) is engineered to have an altered Sialic acid-binding immunoglobulin-type lectin (Siglec) ligand (SigL) profile.

Thus, disclosed herein are engineered autoantigens which may retain the epitope(s) recognized by autoimmune antibodies, while comprising one or more modifications comprising addition of a SigL that render the autoantigen capable of suppressing an antigen-specific immune response in an individual to which it has been administered as compared to the unmodified autoantigen. In some embodiments, the immune response is a humoral immune response i.e., a B cell-driven response, e.g., an IgG response.

Disclosed herein is an engineered autoantigen, comprising an autoantigen which has been engineered to comprise a modified SigL profile relative to a corresponding unengineered autoantigen (e.g., native autoantigen) while the autoantigen comprises an epitope recognized by an autoimmune antibody, or multiple epitopes recognized by several autoimmune antibodies, as described above. By a “Siglec” it is meant a member of the family of proteins that are found primarily on the surface of leukocytes and that bind sialic acids. By a “Siglec ligand profile”, it is meant the amount and/or location of Siglec ligands that are covalently bound to an autoantigen. In some instances, the engineered autoantigen comprises a naturally occurring sialic acid, where the naturally occurring sialic acid is covalently bound to the autoantigen. In many instances, the modification is an increase in the amount and/or change of location of Siglec ligands that are covalently bound to an autoantigen, wherein the increase and/or different location renders the autoantigen able to suppress B cell activation more than the corresponding unmodified autoantigen.

In other instances, the modification is the genetic, in-frame fusion of the autoantigen sequence with sequences (protein-based Siglec binders) that have specific binding activity for Siglec proteins.

There are 14 different mammalian Siglecs, which are expressed on different types of leukocytes and which may exert inhibitory or activating effects on the cells on which they are expressed depending on whether they comprise an inhibitory motif or activating motif. Siglecs show distinct binding preferences for different sialic acids, and the type of linkage and type of underlying sugar also affect recognition of sialic acids. (Varki, A. and Crocker, P. R. (2009) I-type lectins. In Essentials of Glycobiology (2nd ed) (Varki, A. et al., eds), pp. 459-474, Cold Spring Harbor Laboratory Press; Crocker, P. R. et al. (2007) Nat. Rev. Immunol. 7, 255-266). Together, this provides for an array of alternative Siglec ligands that may be deployed to modulate an immune response to an autoantigen. Of particular interest is the suppression of an immune response to an autoantigen, and more particularly, of a B cell response to the autoantigen. Accordingly, in some embodiments, the Siglec ligand is a ligand for a Siglec that is expressed on B lymphocytes, for example Siglec-2 (also called CD22), Siglec-5 (CD170), Siglec-6, Siglec-9 (CD329), or Siglec-10 (Siglec-G). In some embodiments, the Siglec is Siglec-2 (also called CD22). In some embodiments, the Siglec is Siglec-5. In some embodiments, the Siglec is Siglec-6. In some embodiments, the Siglec is Siglec-9. In some embodiments, the Siglec is Siglec-10. In some embodiments, the autoantigen has been engineered to comprise sialic acid ligand for one Siglec. In other embodiments, the hypoimmunogenic autoantigen has been engineered to comprise Siglec ligands for two or more Siglecs, e.g., for 3 Siglecs or for 4 Siglecs, in certain cases, for 5 Siglecs or more.

In some cases, the engineered autoantigen is of formula (I):

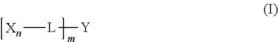

wherein X is a sialic acid group, L is an optional linker, Y is the autoantigen, and n is an integer of 1 or more, and m is an integer of 1 or more. The combination of X and L groups, i.e. [Xₙ-L], is collectively referred to as the Siglec ligand herein. In certain aspects, Y is not a biotherapeutic. In contrast to a biotherapeutic, a subject produces an endogenous form of the autoantigen, while the subject does not produce a biotherapeutic. Further, immune response to an autoantigen is an autoimmune response whereas immune response to a biotherapeutic is a normal immune response that a subject normally mounts against a foreign antigen. Another distinction between Y and a biotherapeutic is that a biotherapeutic has a primary pharmacology (i.e., clinical benefit, e.g., TNFalpha neutralization, Her2+ tumor cell killing) while autoantigen Y has no inherent intended pharmacology (i.e., clinical benefit) on its own. The primary pharmacology of the engineered autoantigen is of the entire SigL-Autoantigen structure, where that pharmacology is the specific suppression or deletion of autoantigen-specific B cell clones.

As discussed in the context of method for treating an individual having an immune response to an antigen, the engineered antigen administered to the subject may be Y as defined in formula (I). In certain aspects, Y may be an autoantigen or a biotherapeutic.

### Sialic Acid Group, X

X is a sialic acid group, wherein the term “sialic acid” refers to alpha-keto acid sugars with a nine-carbon backbone. Thus, since X is a sialic acid group, X comprises a sialic acid or a derivative thereof. The sialic acid or derivative thereof can be naturally occurring or non-naturally occurring. In some cases, X comprises neuraminic acid, which is one example of a sialic acid, or a derivative thereof.

In some embodiments, the sialic acid is a naturally occurring sialic acid. The sialic acid family comprises approximately 50 naturally occurring members. Most common amongst these are N-acetylneuraminic acid (Neu5Ac), N-glycolylneuraminic acid (Neu5Gc, enzymatically produced from Neu5Ac by adding a single oxygen atom (i.e., hydroxylation)), 2-keto-3 deoxynonulosonic acid (Kdn), and neuraminic acid (Neu); others are well known in the art, as reviewed in, e.g., Schauer (2000) Glycoconjugate J 17:485-499. Thus, for example, when the Siglec to be targeted is CD22, the Siglec ligand may be a naturally occurring CD22 ligand, i.e. α2,6-linked sialic acid such as Neu5Acα2-6Galβ-4GlcNAc-6S; when the Siglec is Siglec-5, the Siglec ligand may be a naturally occurring Siglec-5 ligand, i.e. Neu5Aca8-8Neu5Ac and Neu5Aca2-6GaINAc; when the Siglec is Siglec-6, the Siglec ligand may be a naturally occurring Siglec-6 ligand, i.e. Neu5Aca2-6GaINAc; when the Siglec is Siglec-9, the Siglec ligand may be a naturally occurring Siglec-9 ligand, i.e. Neu5Aca2-3Galβ-4GlcNAc-6Sα-3fucose; or when the Siglec is Siglec-10/G, the Siglec ligand may be a naturally occurring Siglec-10 ligand, i.e. α2,6-linked sialic acid or α2,3-linked sialic acid, such as Neu5Aca2-6Galβ-4GlcNAc (O'Reilly, M. K. and Paulson, J. C. (2009) Trends Pharmacol. Sci. 30, 240-248). In some embodiments, the hypoimmunogenic autoantigen has been engineered to comprise the sialic acid ligands for two or more Siglecs, e.g., for 3 Siglecs or for 4 Siglecs, in certain cases, for 5 Siglecs, or more.

In some embodiments, the sialic acid is a non-naturally occurring, i.e. synthetic, sialic acid. A “synthetic sialic acid” also known in the art and referred to herein as a “sialic acid mimetic” or “SAM”, refers to a sialic acid that does not occur in nature, i.e., an alpha-keto acid sugar derivative comprising a nine-carbon backbone that is non-naturally occurring. Compared with Siglec ligands comprising natural sialic acids, which have weak monovalent binding affinities for Siglecs (0.1-3 mM), Siglec ligands comprising SAMs can feature binding affinities in the nanomolar range (Crocker, P. R. et al. (2007) Nat. Rev. Immunol. 7, 255-266; Prescher, H. et al. (2014) ACS Chem. Biol. 9, 1444-1450). SAMs that find use in the subject compositions include those in which one or more positions of a natural sialic acid ranging from the aglycone (C-2) to the rest of the backbone (C-3 to C-9) have been modified to improve Siglec binding. For example, the modifications C-9-NH2 (9-NH2-Neu5Ac/Me) and C-5-FAc (Neu5FAc/Me) improve Siglec-2 binding due to an increase in hydrogen bonding and lipophilic interactions between the SAM and Siglec-2, and incorporating a lipophilic group has since been used to rationally design additional SAMs having an increased binding affinity for Siglec-2 (van Rossenberg, S. M. W. et al. (2001) J. Biol. Chem. 276, 12967-12973; Kelm, S. et al. (2002) J. Exp. Med. 195, 1207-1213; Zaccai, N. R. et al. (2003) Structure 11, 557-567). Other nonlimiting examples of SAMs that find use in the present application include 9-N-biphenylcarboxyl-NeuAcα2-Galβ1-4GlcNAc (6′-BPCNeuAc), NeuAcα2-6Galβ1-4GlcNAc, NeuAcα2-6Galβ1-4(6-sulfo)GlcNAc; those SAMs disclosed in Bull et al. (2016) Sialic Acid Mimetics to Target the Sialic Acid-Siglec Axis. Trends Biochem Sci. 41(6):519-531 and Prescher, H. et al. (2014) Discovery of multifold modified sialosides as human CD22/Siglec-2 ligands with nanomolar activity on B-cells. ACS Chem. Biol. 9, 1444-1450; and those SAMs disclosed in U.S. Pat. Nos. 8,357,671, 9,522,183, 9,981,023, the full disclosures of which are incorporated herein by reference. In certain embodiments, the SAM is a SAM provided in Table 1 below.

TABLE 1

| Siglec | SAM | IC50 | Reference |
|---|---|---|---|
| Siglec- 2 (CD22) | 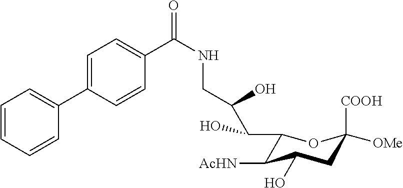 | 4 μM | Kelm, S. et al. J. Exp. Med. 2002 |
| | 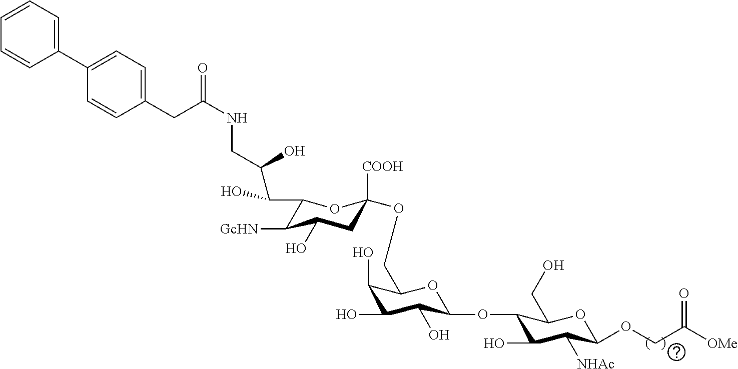 | 2.1 μM | Collins, BE et al. J. Immunol 2006 |
| | 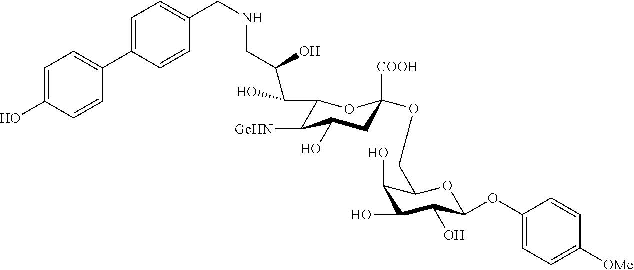 | 0.24 μM | Abdu- Allah, HHM et al. J. Med Chem 2008 |
| | 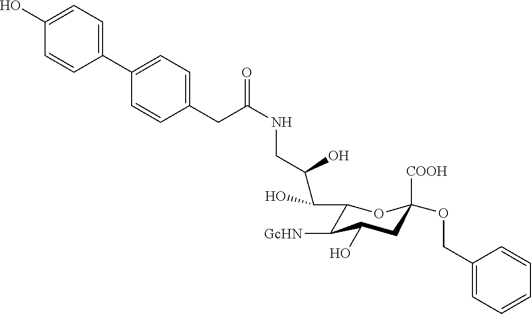 | 0.10 μM | Abdu- Allah, HHM et al. Bioorg. Med. Chem Lett 2009; Abdu- Allah, HHM et al. Bioorg Med Chem 2011 |
| | 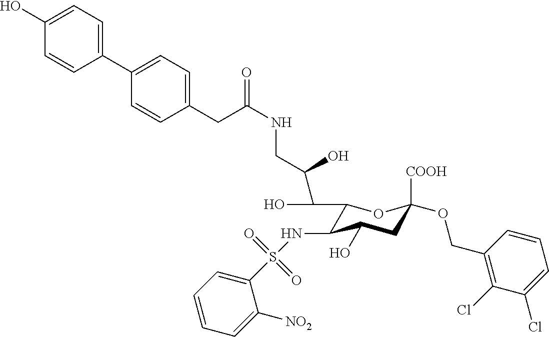 | 0.06 μM | Mesch S et al. Chem Med Chem 2012 |
| | 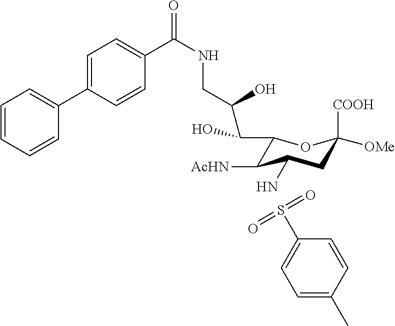 | 0.15 μM | Kalm S. et al. Angew. Chem. Int. Ed. Engl 2012 |
| | 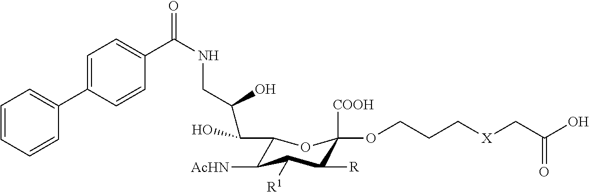 | 6 nM or 2 nM | Prescher, H. et al. ACS Chem. Biol. 2014 |
| | R = OH, R¹ = NH(CO)Et, X = CH₂, IC50 = 6 nm | | |
| | R = H, R¹ = OSO₃Na, X = SCH₂, IC50 = 2 nm [S1] | | |
| Siglec- 5 (CD170) | 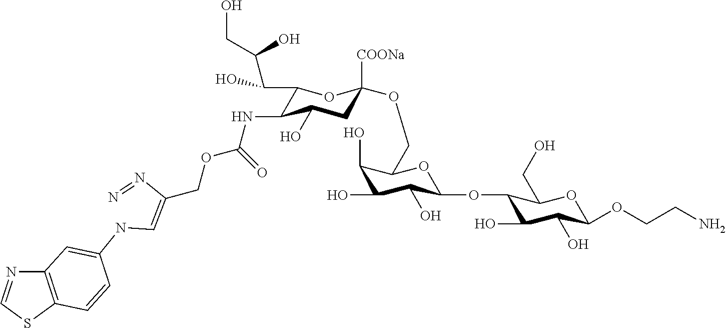 | NA | Rillahan, CD et al. Angew. Chem. Int. Ed. Engl 2012 |
| Siglec- 9 (CD329) | 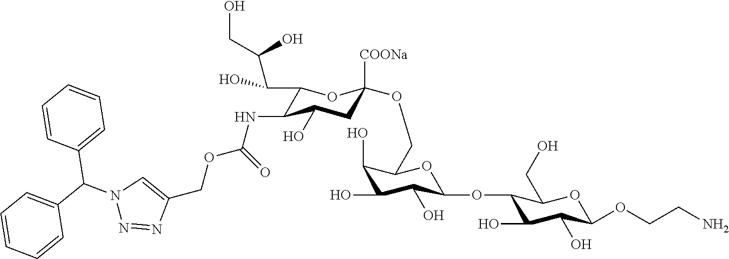 | NA | Rillahan, CD et al. Angew. Chem. Int. Ed. Engl 2012 |
| Siglec- 10 (Siglec- G) | 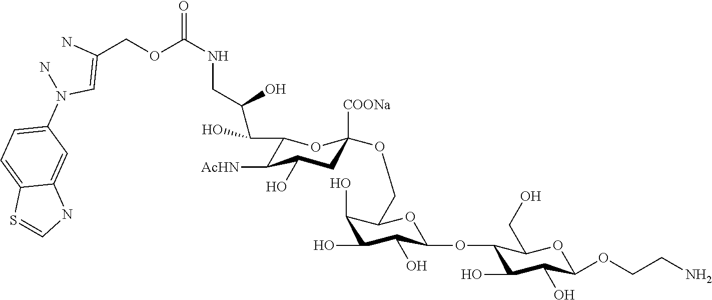 | NA | Rillahan, CD et al. Angew. Chem. Int. Ed. Engl 2012 |

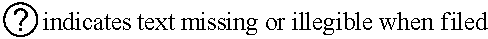

As described in formula (I) above, n is an integer of 1 or more, such as an integer from 1 to 20, or from 1 to 15, or from 1 to 10, or from 1 to 5. In some cases, n is 1, 2, 3, 4, or 5. In some cases, n is 1. In some cases, n is 2. In some cases, n is 3. In some cases, n is 4. In some cases, n is 5.

If more than one X is present, i.e., if n is greater than 1, then the X groups can be the same or different from each other. If L is present, then each X is directly covalently bonded to L, and L is directly covalently bonded to Y. If L is absent, then each X is directly covalently bonded to Y. In some cases, n is 1. In some cases, n is an integer of 2 or more, such as 2, 3, 4, 5, 6, 7, 8, 9, or 10.

As stated above, m is an integer of 1 or more. In some cases, m is 2 or more, 3 or more, 5 or more, or 10 or more. In some cases, m ranges from 1 to 20, such as from 2 to 10.

In some embodiments, the sialic acid group, X, has a structure of Formula (II):

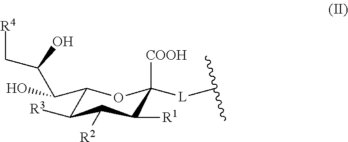

In some embodiments, R¹, R², R³ and R⁴ are each independently selected from hydrogen, alkyl, substituted alkyl, alkenyl, substituted alkenyl, alkynyl, substituted alkynyl, hydroxyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, cycloalkyl, substituted cycloalkyl, heterocyclyl, and substituted heterocyclyl, aryl, substituted aryl, heteroaryl, and substituted heteroaryl.

In some embodiments, L is a linker as described herein.

### Linker, L

L is an optional linker. As such, in some cases the engineered autoantigen has the linker, and the engineered autoantigen can be described by the formula [Xₙ-L]ₘ-Y. In other cases, the engineered autoantigen does not have the linker, and the engineered autoantigen can be described by the formula [Xₙ]ₘ-Y.

Embodiments of the engineered autoantigen can be used to demonstrate possible configurations of [Xₙ-L]ₘ-Y. As described above, X is a sialic acid group comprising a sialic acid or a derivative thereof. In the compound shown below, a single neuraminic acid group is present, corresponding to a single X group. Hence, in the embodiment below, n is 1. The group shown to the left of the neuraminic acid with the formula (phenyl)-C(O)-phenylene- can be considered to be a part of the X group. Although the autoantigen Y is not shown in the compound, the —C(O)—O—C₆F₅ group can undergo a chemical reaction that forms a covalent bond with a autoantigen Y.

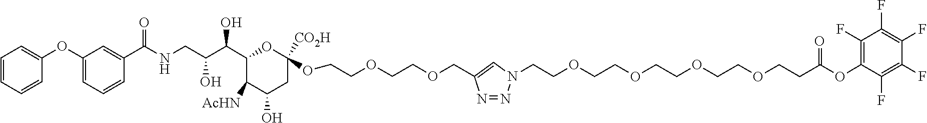 [SMILES: CC(=O)N[C@H]1[C@H]([C@H](O)[C@H](O)CNC(=O)c2cccc(Oc3ccccc3)c2)O[C@](OCCOCCOCc2cn(CCOCCOCCOCCOCCC(=O)Oc3c(F)c(F)c(F)c(F)c3F)nn2)(C(=O)O)C[C@@H]1O]

In the embodiment shown below, three neuraminic acid groups are shown, indicating that there are three X groups, and thus n is 3. In addition, the three X groups are covalently bonded to each other through a branching group comprising derivatives of lysine residues. This branching group is part of linker L. Stated in another manner, this embodiment includes the optional linker L, wherein L is a branching group that covalently connects the three X groups to one another. Linker L also covalently connects the X groups to the —C(O)—O—C₆F₅ group that can undergo a chemical reaction that forms a covalent bond with an autoantigen Y. Hence, linker L also covalently links the X groups to the autoantigen Y.

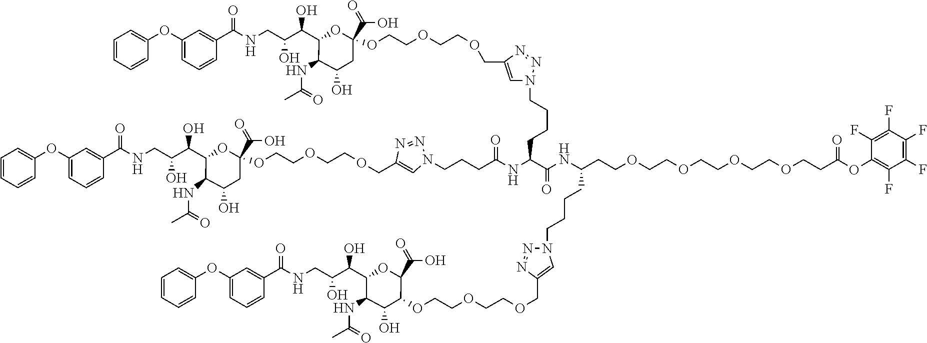 [SMILES: CC(=O)N[C@@H]1[C@@H](O)[C@@H](OCCOCCOCc2cn(CCCC[C@@H](CCOCCOCCOCCOCCC(=O)Oc3c(F)c(F)c(F)c(F)c3F)NC(=O)[C@H](CCCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)NC(=O)CCCn3cc(COCCOCCO[C@]4(C(=O)O)C[C@H](O)[C@@H](NC(C)=O)[C@H]([C@H](O)[C@H](O)CNC(=O)c5cccc(Oc6ccccc6)c5)O4)nn3)nn2)[C@H](C(=O)O)O[C@H]1[C@H](O)[C@H](O)CNC(=O)c1cccc(Oc2ccccc2)c1]

As described above, the combination of X and L groups is collectively referred to as a Siglec ligand. In other words, [Xₙ-L] is a Siglec ligand.

In some embodiments, each sialic acid group of each X is covalently connected to an autoantigen Y through a chain of atoms that does not include a sugar group. For instance, each X group includes a single sialic acid group but does not include any other sugar groups. Furthermore, in some embodiments, if L is present, L directly covalently connects each X to Y and L does not comprise a sugar group. The term “sugar” as used herein refers to monosaccharides and disaccharides. As such, even if L includes a trisaccharide, such a L would not be within the scope of such embodiments because a trisaccharide includes a disaccharide unit and a monosaccharide unit. Stated in another manner, the only sugar groups present in [Xₙ-L] is a single sialic acid group for each X. No sialic acid groups are directly covalently bonded to one another.

In some embodiments, each sialic acid group of each X is covalently connected to an autoantigen Y through a chain of atoms that does not include an oxygen-containing heterocyclic group. Notably, monosaccharide sugars such as glucose and galactose are heterocyclic groups containing an oxygen atom in the ring. In some embodiments, each sialic acid group of each X is covalently connected to an autoantigen Y through a chain of atoms that consists of one or more chemical moieties selected from the group consisting of: alkyl, alkenyl, alkynyl, polyethylene glycol, aryl, heteroaryl, sulfur atom-containing heterocycle, nitrogen atom-containing heterocycle, amino acid residue, amino, acyl, halo, hydroxy, carboxy, sulfoxy, and substituted analogs thereof.

In some embodiments, if present, linker L consists of one or more chemical moieties selected from the group consisting of: alkyl, alkenyl, alkynyl, polyethylene glycol, aryl, heteroaryl, sulfur atom-containing heterocycle, nitrogen atom-containing heterocycle, amino acid residue, amino, acyl, halo, hydroxy, carboxy, sulfoxy, and substituted analogs thereof. In addition, the section of X between the sialic acid and L or Y consists of one or more chemical moieties selected from the group consisting of: alkyl, alkenyl, alkynyl, polyethylene glycol, aryl, heteroaryl, sulfur atom-containing heterocycle, nitrogen atom-containing heterocycle, amino acid residue, amino, acyl, halo, hydroxy, carboxy, sulfoxy, and substituted analogs thereof.

In some cases wherein L is present, L is a branched linker. In other words, L directly covalently connects an autoantigen Y to two or more X groups. In some cases, a branching location of L includes an amino acid residue or a derivative thereof, e.g. lysine or a derivative thereof. For instance, the branching location of L can have the formula shown below, wherein each location marked with an asterisk (*) is a site for heading towards an X group or the Y group.

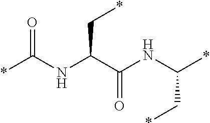 [SMILES: *C[C@@H](*)NC(=O)[C@H](C*)NC(*)=O]

In some cases, the branching location of L does not comprise an aryl group or a heteroaryl group. In some cases, the branching location of L comprises an alkyl group, an amide group, an amino acid residue group, or a combination thereof.

In some embodiments, linker L comprises a polyethylene glycol group, a triazole group, or a combination thereof. In some cases, the section of X between the sialic acid group and L or Y comprises a polyethylene glycol group, a triazole group, or a combination thereof. In some cases, the triazole group is part of a covalent connection between the X and L groups.

In some embodiments, the linker, L, can include one or more linker subunits (LS), such as 2, 3, 4, 5, 6, 7, 8, 9 or 10, or even more linker subunits (LS). For example, some embodiments of the linker can include 1 to 10 linker subunits (LS) described by Formula (III):

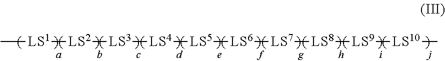

where LS¹, LS², LS³, LS⁴, LS⁵, LS⁶, LS⁷, LS⁸, LS⁹ and LS¹⁰ are each independently a linker subunit, and a, b, c, d, e, f, g, h, i and j are each independently 0 or 1. In some embodiments, the sum of a to j is 1 (e.g., a is 1 and b to j are each 0). In these embodiments, the linker subunit LS¹ is attached at one end to Y and at the other end to X. In some embodiments, the sum of a to j is 2 (e.g., a and b are each 1, and c to j are each 0). In these embodiments, the linker subunit LS¹ is attached to Y and the linker subunit LS² is attached to X. In some embodiments, the sum of a to j is 3 (e.g., a to c are each 1, and d to j are each 0). In these embodiments, the linker subunit LS¹ is attached to Y and the linker subunit LS³ is attached to X. In some embodiments, the sum of a to j is 4 (e.g., a to d are each 1, and e to j are each 0). In these embodiments, the linker subunit LS¹ is attached to Y and the linker subunit LS⁴ is attached to X. In some embodiments, the sum of a to j is 5 (e.g., a to e are each 1, and f to j are each 0). In these embodiments, the linker subunit LS¹ is attached to Y and the linker subunit LS⁵ is attached to X. In some embodiments, the sum of a to j is 6 (e.g., a to f are each 1, and g to j are each 0). In these embodiments, the linker subunit LS¹ is attached to Y and the linker subunit LS⁶ is attached to X. In some embodiments, the sum of a to j is 7 (e.g., a to g are each 1, and h to j are each 0). In these embodiments, the linker subunit LS¹ is attached to Y and the linker subunit LS⁷ is attached to X. In some embodiments, the sum of a to j is 8 (e.g., a to h are each 1, and i and j are each 0). In these embodiments, the linker subunit LS¹ is attached to Y and the linker subunit LS⁹ is attached to X. In some embodiments, the sum of a to j is 9 (e.g., a to i are each 1, and j is 0). In these embodiments, the linker subunit LS¹ is attached to Y and the linker subunit LS⁹ is attached to X. In some embodiments, the sum of a to j is 10 (e.g., a to j are each 1). In these embodiments, the linker subunit LS¹ is attached to Y and the linker subunit LS¹⁰ is attached to X.

Any convenient functional group can be used in each linker subunit (LS) in the linker. In some embodiments, a linker subunit (LS) may include a group selected from, but not limited to, alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl.

In some embodiments, a linker subunit includes a functional group independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, substituted heterocyclyl, (PEG)ₙ, and (AA)ₚ, where each n is independently an integer from 1 to 50 and each p is independently an integer from 1 to 20. As used herein, “PEG” refers to polyethylene glycol.

As used herein, “AA” refers to an amino acid residue. Amino acid residues include amino acids commonly found in naturally occurring proteins (e.g., Ala or A, Cys or C, Asp or D, Glu or E, Phe or F, Gly or G, His or H, lie or I, Lys or K, Leu or L, Met or M, Asn or N, Pro or P, Gln or Q, Arg or R, Ser or S, Thr or T, Val or V, Trp or W, Tyr or Y). In some embodiments, amino acid residues used in the linkers and linker subunits described herein also include amino acid analogs and amino acid derivatives, which are natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or attachment of another group to the side chain or backbone, or combinations thereof. For example, amino acid analogs may include α-hydroxy acids, and α-amino acids, and the like. In some instances, an amino acid analog or amino acid derivative can include another group, such as another sialic acid moiety (X), attached to the side chain or backbone of the amino acid analog or amino acid derivative through an optional linker.

In some embodiments, a linker subunit includes a functional group independently selected from (C₁-C₁₂)alkyl or substituted (C₁-C₁₂)alkyl. In some embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂).

In some embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene. In some instances, substituted (C₁-C₁₂)alkyl is a substituted C₂-alkylene.

In some embodiments, a linker subunit includes a functional group independently selected from aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from aryl or substituted aryl. In some instances, the linker subunit includes an aryl. For example, the aryl can be phenyl. In some instances, the linker subunit includes a substituted aryl. In some cases, the substituted aryl is a substituted phenyl. The substituted aryl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from heteroaryl or substituted heteroaryl. In some cases, the linker subunit includes a heteroaryl. In some cases, the linker subunit includes a substituted heteroaryl. The substituted heteroaryl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from cycloalkyl or substituted cycloalkyl. In some cases, the linker subunit includes a cycloalkyl. In some cases, the linker subunit includes a substituted cycloalkyl. The substituted cycloalkyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some instances, a linker subunit includes a functional group independently selected from heterocyclyl or substituted heterocyclyl. In some cases, the linker subunit includes a heterocycloalkyl. For example, the linker subunit can include a triazole (e.g., 1,2,3-triazole). In some cases, the linker subunit includes a substituted heterocycloalkyl. The substituted heterocyclyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl.

In some embodiments, the linker does not include a natural saccharide.

In some embodiments, the linker (L) includes one or more tether groups adjacent to or in between one or more linker subunits (LS) in the linker. The tether groups may facilitate attachment between two linker subunits, between a linker subunit and a reactive termini for conjugation to the moiety of interest (Y), or between a linker subunit and the sialic acid moiety (X). The tether groups may include convenient functional groups that facilitate these attachments, such as, but not limited to, amino, carbonyl, amido, oxycarbonyl, carboxy, thioether, sulfonyl, sulfoxide, sulfonylamino, aminosulfonyl, thio, oxy, phospho, phosphoramidate, thiophosphoraidate, and the like. In some embodiments, the tether groups are each independently selected from a covalent bond, —CO—, —NR¹⁵—, —NR¹⁵(CH₂)_{q}—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In some embodiments, q is an integer from 1 to 6 (e.g., 1, 2, 3, 4, 5 or 6). In some embodiments, q is 1. In some embodiments, q is 2. In some embodiments, q is 3. In some embodiments, q is 4. In some embodiments, q is 5. In some embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, R¹⁵ is hydrogen. In some embodiments, each R¹⁵ is hydrogen. In some embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In some embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In some embodiments, R¹⁵ is alkynyl or substituted alkynyl. In some embodiments, R¹⁵ is alkoxy or substituted alkoxy. In some embodiments, R¹⁵ is amino or substituted amino. In some embodiments, R¹⁵ is carboxyl or carboxyl ester. In some embodiments, R¹⁵ is acyl or acyloxy. In some embodiments, R¹⁵ is acyl amino or amino acyl. In some embodiments, R¹⁵ is alkylamide or substituted alkylamide. In some embodiments, R¹⁵ is sulfonyl. In some embodiments, R¹⁵ is thioalkoxy or substituted thioalkoxy. In some embodiments, R¹⁵ is aryl or substituted aryl, such as C₅_₈ aryl or C₅_₈ substituted aryl, such as a C₅ aryl or C₅ substituted aryl, or a C₆ aryl or C₆ substituted aryl. In some embodiments, R¹⁵ is heteroaryl or substituted heteroaryl, such as C₅_₈ heteroaryl or C₅_₈ substituted heteroaryl, such as a C₅ heteroaryl or C₅ substituted heteroaryl, or a C₆ heteroaryl or C₆ substituted heteroaryl. In some embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In some embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₈ substituted heterocyclyl.

In some embodiments, a linker subunit (LS) may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In some embodiments, the polymer is a polyalkylene glycol. In some embodiments, the polymer is a polyethylene glycol (PEG).

In some cases, the linker is not branched and connects one X group to the Y group, and thus may be referred to as monovalent. In some cases, the linker is a branched linker that is divalent and connects two X groups to the Y group. In certain cases, the linker is a branched linker that is trivalent and connects three X groups to the Y group. In some instances, the linker is a branched linker of a higher multivalency and connects multiple X groups to the Y group. In some cases, the linker has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms of the cyclic group are included in the backbone.

In some embodiments, a linker subunit (LS) may be a branched subunit. A branched subunit may be a linker subunit that is attached to two or more sialic acid moieties, either directly or through a respective linker for each sialic acid moiety. For example, a branched subunit may be attached to two sialic acid moieties. In some embodiments, a branched subunit includes an amino acid (AA). For instance, a branched subunit may include an amino acid where the backbone of the amino acid forms part of the linker attached to a first sialic acid moiety and where the side chain of the amino acid is conjugated to a second sialic acid moiety either directly or through a linker of the branch (i.e., “a branch linker”). For example, in some embodiments, a branched subunit includes a lysine where the backbone of the lysine forms part of the linker attached to a first sialic acid moiety and where the side chain of the lysine is conjugated to a second sialic acid moiety either directly or through a branch linker. In some embodiments, the second sialic acid moiety is conjugated to the lysine by attachment at the terminal amine of the lysine side chain. In some embodiments, the branch linker is a linker as described by Formula (II) above.

Examples of linkers according to the present disclosure include, but are not limited to, the following:
LS¹ is PEG (e.g., PEG₄); LS² is heterocyclyl (e.g., 1,2,3-triazole); LS³ is PEG (e.g., PEG₂); and d to j are each 0;
LS¹ is PEG (e.g., PEG₄); LS² is a branched subunit (e.g., lysine); LS³ is alkyl (e.g., C₃-alkyl); LS⁴ is PEG (e.g., PEG₂); LS¹ is heterocyclyl (e.g., 1,2,3-triazole); LS⁶ is PEG (e.g., PEG₂); and g to j are each 0; and where the LS² branched subunit is attached to a branch linker described by the following: LS¹ is PEG (e.g., PEG₂); LS² is heterocyclyl (e.g., 1,2,3-triazole); LS³ is PEG (e.g., PEG₂); and d to j are each 0;
LS¹ is PEG (e.g., PEG₄); LS² is heterocyclyl (e.g., 1,2,3-triazole); LS³ is alkyl (e.g., C₂-alkyl); and d to j are each 0;
LS¹ is PEG (e.g., PEG₄); LS² is a branched subunit (e.g., lysine); LS³ is alkyl (e.g., C₃-alkyl); LS⁴ is PEG (e.g., PEG₂); LS¹ is heterocyclyl (e.g., 1,2,3-triazole); LS⁶ is alkyl (e.g., C₂-alkyl); and g to j are each 0; and where the LS² branched subunit is attached to a branch linker described by the following: LS' is PEG (e.g., PEG₂); LS² is heterocyclyl (e.g., 1,2,3-triazole); LS³ is alkyl (e.g., C₂-alkyl); and d to j are each 0;
LS¹ is PEG (e.g., PEG₄); LS² is a branched subunit (e.g., lysine); LS³ is a branched subunit (e.g., lysine); LS⁴ is alkyl (e.g., C₃-alkyl); LS¹ is PEG (e.g., PEG₂); LS⁶ is heterocyclyl (e.g., 1,2,3-triazole); LS¹ is alkyl (e.g., C₂-alkyl); and h to j are each 0; where the LS² branched subunit is attached to a branch linker described by the following: LS¹ is PEG (e.g., PEG₂); LS² is heterocyclyl (e.g., 1,2,3-triazole); LS³ is alkyl (e.g., C₂-alkyl); and d to j are each 0; and where the LS³ branched subunit is attached to a branch linker described by the following: LS¹ is PEG (e.g., PEG₂); LS² is heterocyclyl (e.g., 1,2,3-triazole); LS³ is alkyl (e.g., C₂-alkyl); and d to j are each 0;
LS¹ is PEG (e.g., PEG₄); LS² is a branched subunit (e.g., lysine); LS³ is a branched subunit (e.g., lysine); LS⁴ is alkyl (e.g., C₃-alkyl); LS¹ is PEG (e.g., PEG₂); LS⁶ is heterocyclyl (e.g., 1,2,3-triazole); LS¹ is PEG (e.g., PEG₂); and h to j are each 0; where the LS² branched subunit is attached to a branch linker described by the following: LS¹ is PEG (e.g., PEG₂); LS² is heterocyclyl (e.g., 1,2,3-triazole); LS³ is PEG (e.g., PEG₂); and d to j are each 0; and where the LS³ branched subunit is attached to a branch linker described by the following: LS¹ is PEG (e.g., PEG₂); LS² is heterocyclyl (e.g., 1,2,3-triazole); LS³ is PEG (e.g., PEG₂); and d to j are each 0;
LS¹ is a branched subunit (e.g., lysine); LS² is a branched subunit (e.g., lysine); LS³ is alkyl (e.g., C₃-alkyl); LS⁴ is heterocyclyl (e.g., 1,2,3-triazole); LS¹ is PEG (e.g., PEG₂); and f to j are each 0; where the LS¹ branched subunit is attached to a branch linker described by the following: LS¹ is heterocyclyl (e.g., 1,2,3-triazole); LS² is PEG (e.g., PEG₂); and c to j are each 0; and where the LS² branched subunit is attached to a branch linker described by the following: LS¹ is heterocyclyl (e.g., 1,2,3-triazole); LS² is PEG (e.g., PEG₂); and c to j are each 0;
LS¹ is PEG (e.g., PEG₄); LS² is a branched subunit (e.g., lysine); LS³ is a branched subunit (e.g., lysine); LS⁴ is alkyl (e.g., C₃-alkyl); LS¹ is heterocyclyl (e.g., 1,2,3-triazole); LS⁶ is PEG (e.g., PEG₂); and g to j are each 0; where the LS² branched subunit is attached to a branch linker described by the following: LS¹ is heterocyclyl (e.g., 1,2,3-triazole); LS² is PEG (e.g., PEG₂); and c to j are each 0; and where the LS³ branched subunit is attached to a branch linker described by the following: LS¹ is heterocyclyl (e.g., 1,2,3-triazole); LS² is PEG (e.g., PEG₂); and c to j are each 0; and
LS¹ is PEG (e.g., PEG₄); LS² is a branched subunit (e.g., lysine); LS³ is alkyl (e.g., C₃-alkyl); LS⁴ is heterocyclyl (e.g., 1,2,3-triazole); LS¹ is PEG (e.g., PEG₂); and f to j are each 0; and where the LS² branched subunit is attached to a branch linker described by the following: LS¹ is heterocyclyl (e.g., 1,2,3-triazole); LS³ is PEG (e.g., PEG₂); and c to j are each 0.

The linkers described above may include one or more tether groups to facilitate attachment between two linker subunits, between a linker subunit and a reactive termini for conjugation to the moiety of interest (Y), or between a linker subunit and the sialic acid moiety (X).

Siglec Ligand [Xₙ-L]ₘ

In the formula [Xₙ-L]ₘ-Y, [Xₙ-L] represent the Siglec ligand, and m represent an integer from 1-25. It is conceived that one or more Siglec ligands, e.g. 2, 3, 4, or 5 or more Siglec ligand, in some cases 6, 7, 8, 9, or 10 or more Siglec ligands, in some such cases 11, 12, 13, 14, 15 or more Siglec ligands, in some cases 16, 17, 18, 19, 20 or more Siglec ligands, sometimes 21, 22, 23, 24 or 25 Siglec ligands, may be conjugated to the autoantigen, either appended to the same or to different amino acids of the autoantigen. In some embodiments, the Siglec ligand comprises a sialic acid. The Siglec ligand can be naturally occurring, i.e., a moiety comprising a naturally occurring sialic acid and a naturally occurring glycan, wherein the sialic acid and glycan are typically found in nature in association with one another to form a Siglec ligand. The Siglec ligand can be non-naturally occurring, e.g., a moiety comprising a naturally occurring sialic acid and a linker, a moiety comprising a non-naturally occurring sialic acid and a glycan found in nature as part of a Siglec ligand, a moiety comprising a non-naturally occurring sialic acid and a linker, a moiety comprising a peptide or protein having an affinity for a Siglec, and the like.

### Autoantigen, Y

In the formula [Xₙ-L]ₘ-Y, Y is the autoantigen. Y by itself is referred to as an unengineered autoantigen, which term is used interchangeably with the term parental autoantigen. However, the combination of elements that is [Xₙ-L]ₘ-Y is referred to as the engineered autoantigen. Y, in and of itself, can be a naturally occurring protein. Y, in some instances, is an enzyme, intercellular adhesive protein, cell junction protein, cytoskeletal protein, extracellular matrix protein, cellular receptor, transcription or translational protein, gene editing protein, structural protein, or the like. Y is generally naturally present in all individuals; however, some individuals may develop an autoimmune disease where the individuals produce antibodies that bind to Y.

In some embodiments, [Xₙ-L] provides a therapeutic activity to the protein. Stated in another way, the [Xₙ-L] enables the autoantigen Y to function in-vivo by treating an autoimmune disease in a subject who is producing antibodies against the autoantigen.

Any protein, glycan, lipid, or nucleic acid autoantigen may serve as the autoantigen that is engineered to include a SigL according to the present disclosure, including, for example, a protein, e.g., enzyme, intercellular adhesive protein, cell junction protein, cytoskeletal protein, extracellular matrix protein, cellular receptors, transcription or translational protein, gene editing protein, structural protein, or the like. In certain embodiments, an autoantigen may be a naturally occurring protein.

The autoantigen may be an engineered protein, for example, an engineered DSG-3 and/or DSG-1, an engineered Gliadin and type 2 transglutaminase, an engineered PDC-E2, an engineered PLA2R (Phospholipase A2 Receptor) to which a siglec ligand has been conjugated.

In some embodiments, the engineered autoantigen is a variant of a naturally occurring protein. By “variant” it is meant a mutant having deletions and/or substitutions relative to the native autoAg. In certain aspects, the engineered autoantigen comprises an amino acid sequence having less than 100% sequence identity to the native autoAg. For example, the engineered autoantigen may comprise an amino acid sequence having 10%, 20%, 30%, 40%, 50%, 60% sequence identity or more with a full-length native protein, e.g., 65%, 70%, 75%, or 80% or more identity, such as 85%, 90%, or 95% or more identity, for example, 98% or 99% identity with the full-length native protein. Variants also include fragments of naturally occurring proteins, particularly those having one or more epitopes to which autoimmune antibodies bind. In certain embodiments, a fragment of an autoantigen may be generated. The fragment may include one or more immunodominant epitopes present in the autoantigen. The fragment may include a contiguous amino acid sequence present in the autoantigen or may be a fusion of two or more non-contiguous amino acid sequences present in the autoantigen, e.g., amino acid sequences from different regions of the autoantigen. The autoantigen sequence may be derived from any source, e.g., human or non-human.

In some embodiments, the engineered autoantigen may include a variant of PLA2R, where the variant PLA2R includes an immunodominant region of a naturally-occurring PLA2R. Primary membranous nephropathy (pMN) is a common nephrotic syndrome and an example of an autoimmune disease driven by a well-defined autoantigen (Couser, W. G. (2017) Clin. J. Am. Soc. Nephrol. 12, 983-997). The kidney podocyte membrane protein PLA2R is known to be a key disease-driving autoantigen for pMN, with approximately 70% of patients showing circulating IgG autoantibodies and another 15% of patients who are seronegative but with positive biopsy staining specific for PLA2R (Beck, L. H., et al., (2009) New. Engl. J. Med. 361, 1). High levels of PLA2R autoantibodies predict poor spontaneous MN remission rates (Timmermans, S. A. M. E. G., et al. (2015) Am. J. Nephrol. 42, 72-77). In patients treated with the B cell-depleting antibody, Rituximab, PLA2R antibody titers are predictive of clinical response, and depletion of PLA2R antibodies is a predictor of therapeutic success (Ruggenenti, et al. (2015) J. Am. Soc. Nethrol. 25, 2545-2558).

The PLA2R protein is a membrane receptor containing a large extracellular region of 10 domains (CysR-FNII-CTLD1-CTLD2-CTLD3-CTLD4-CTLD5-CTLD6-CTLD7-CTLD8) that contains most or all epitopes for PLA2R-reactive antibodies in pMN patients. Immunodominant epitopes of PLA2R have been mapped to the three N-terminal domains (CysR, FnII, and CTLD1) of the extracellular region (Kao, et al. (2015) J Am. Soc. Nephrol. 26, 291-301). The fifth domain, CTLD3, has been identified as immunogenic in an independent study (Fresquet, M., et al. (2015) J. Am. Soc. Nephrol. 26, 302-313). 90% of serum samples from 43 pMN patients showed reactivity with a region of PLA2R containing domains 1 to 5 (CyR-FnII-CTLD1-CTLD2-CTLD3), thus indicating this N-terminal portion of the autoantigen as immunodominant in patients (Fresquet, M., et al. (2015) J. Am. Soc. Nephrol. 26, 302-313). Patients also show epitope spreading, including CTLD1 and the ninth domain, CTLD7, that correlates with poor renal prognosis (Seitz-Polski, et al. (2016) J Am Soc Nephrol 27, 1517-1533).

In certain embodiments, the autoAg-SigL construct disclosed herein may include as the AutoAg part, a portion or the entirety of the 10-domain extracellular region of PLA2R. Such an autoAg-SigL may be used for binding to the clonotypic B cell receptors of disease-driving, PLA2R-specific B cells. Upon engagement of the siglec present on the B cell (e.g., CD22), the construct would suppress and attenuate ongoing autoimmunity to self PLA2R on glomerular podocytes, thus addressing the renal disease pathology of pMN.

In certain embodiments, the autoAg portion of an autoAg-SigL construct provided herein may include one or more of CyR, FnII, CTLD1, CTLD2, and CTLD3 domain of PLA2R. In certain embodiments, the autoAg-SigL construct provided herein is PLA2R CyR-SigL, PLA2R FnII-SigL, PLA2R CTLD1-SigL, PLA2R CTLD2-SigL, PLA2R CTLD3-SigL, PLA2R CyR-FnII-SigL, PLA2R CyR-FnII-CTLD1-SigL, PLA2R CyR-FnII-CTLD1-CTLD2-SigL, or PLA2R CyR-FnII-CTLD1-CTLD2-CTLD3-SigL. In certain embodiments, the autoAg-SigL construct provided herein is PLA2R CTLD1-CTLD7-SigL.

As discussed above, unlike a parental autoantigen that has not been engineered to comprise an altered Siglec ligand profile, the engineered autoantigen of the present disclosure will suppress the development of an immune response to the autoantigen. As such, an engineered autoantigen of the present disclosure may be functionally distinguished from the unengineered, i.e., parental, autoantigen from which it is derived, by assessing the extent to which the engineered autoantigen attenuates the activity of immune cells. By attenuating an activity, it is meant slowing an increase in activity, reducing the activity, or preventing the activity, e.g., by silencing, inhibiting, deleting, etc. the cell or population of cells. Thus, for example, attenuating the activity of a B cell or a population of B cells may include preventing B cells from differentiating into an autoantigen-specific mature B cells, e.g., plasma cells or memory cells, preventing B cells from producing antigen-specific antibodies, preventing the upregulation of activation markers such as CD69, and/or promoting a decrease in viability of an autoantigen-specific B cell population. Typically, the activity of the unengineered, i.e., parental, autoantigen does not include attenuating the activity of an immune cell. More typically, the activity of the unengineered, i.e., parental, autoantigen does not include attenuating the activity of a B cell or a population of B cells.

The ability of an autoantigen engineered according to the present disclosure to suppress an immune response can be readily measured in any number of ways in vitro or in vivo. In vitro, immunosuppression can be measured as, for example, the extent to which a population of B cells is activated by the autoantigen, where less activation is indicative of greater immunosuppression. Any approach known in the art for measuring B cell activation may be used. For example, the extent to which the cells of the population upregulate CD69 expression when contacted with the engineered autoantigen can be assessed, e.g., by measuring the percent of CD69⁺ cells by FACS, by assessing the mean fluorescence intensity (MFI) of the B cells, by assessing the activity of the B cells, and the like. In such analyses it is expected that the engineered autoantigen will activate B cells at least about 2.5-fold less robustly than an unengineered autoantigen, in some instances at least about 5-fold less robustly, at least about 7.5-fold less robustly, or at least about 10-fold less robustly, in some instances about 20-fold less robustly. In vivo, immunosuppression may be measured as, for example, the extent to which the engineered autoantigen elicits “anti-autoantigen antibodies”, or AAAs, relative to the AAAs elicited in an individual, e.g., a mouse injected intramuscularly or intravenously, e.g., in the presence or absence of an immunological adjuvant such as Alum, or, e.g. a human, upon administration of the corresponding unmodified autoantigen, where less AAAs is indicative of greater immunosuppression. In some instances, the anti-autoantigen antibody titer to the engineered autoantigen is reduced by 50% or more relative to the corresponding unmodified autoantigen, for example 60%, 70%, 80% or more, in certain instances 85%, 90%, 95% or more, preferably 98%, 99%, or 100%, i.e., so as to be undetectable. Put another way, the AAA titer that is elicited by the engineered autoantigen can be 50% or less of that which is elicited by a corresponding unengineered autoantigen or less, for example, 40%, 30%, or 20% or less, in certain instances, 15%, 10%, 5% or less, preferably only 2%, 1% or less of that which is elicited by a corresponding unengineered autoantigen.

Methods for detecting antibodies, including but not limited to enzyme-linked immunosorbent assay (ELISA), microparticle ELISA, ELISPOT, radio-immunoprecipitation assays, Electrochemiluminescence immunoassay (ECLIA), DELFIA (dissociation-enhanced lanthanide fluorescence immunoassay), Time-Resolved Fluorescence (TRF) Assay, Surface plasmon resonance immunoassay (SPRIA), Western blotting (immunoblotting), and the like, are well known in the art, any of which may be used to detect antibodies in the serum of an individual to determine if AAAs have been generated. AAA titer may be assessed in an individual's serum following administration of the engineered autoantigen, where the level of AAA detected in serum collected 24 hours or more, e.g. 48 hours or 78 hours or more, in some instances 1, 2, 3, or 4 weeks or more, e.g. 6 weeks or 8 weeks after administration of the engineered autoantigen will be lower than the level of AAAs detected in serum from a control individual treated with the same dosing regimen for the same duration with a corresponding unengineered autoantigen.

As another example, immunosuppression may be observed in vitro as a reduction in leukocyte response upon exposure to the engineered autoantigen relative to a corresponding unengineered autoantigen. For example, greater activation of downstream signaling pathways (e.g., Erk phosphorylation, NFAT nuclear translocation) will be observed in a B cell comprising a CD22 Siglec that is exposed to an unengineered autoantigen as compared to a B cell comprising a CD22 Siglec that is exposed to an autoantigen that has been modified to comprise more CD22 ligand. As yet another example, a CD22 Siglec—and likewise, a cell expressing a CD22 Siglec—will have higher binding affinity for an autoantigen that has been engineered to comprise more CD22 ligand than an unengineered autoantigen.

In some embodiments, the AAAs to the engineered autoantigen are lower in a treated individual's serum after administering the subject autoantigen for one day or more, for example, one month or more, 6 months or more, 9 months or more, or 1 year or more, relative to the level of AAAs detected in serum from a control individual treated with the same dosing regimen and for the same duration with a corresponding unengineered autoantigen. In certain embodiments, the AAAs to the engineered autoantigen are undetectable in an individual's serum after administering the subject autoantigen for one month or more, e.g., 6 months or more, 9 months or more, or 1 year or more, whereas can be detected in serum from a control individual treated with the same dosing regimen and for the same duration with a corresponding unengineered autoantigen. Such administration may be daily, weekly, biweekly, monthly, quarterly, semi-annually, annually, bi-annually, once every 3 years, once every 4 years, once every 5 years, or once every 10 years.

In some cases, when Y is a polypeptide, the polypeptide conjugation reactive terminus of the linker is in some cases a site that is capable of conjugation to the polypeptide through a cysteine thiol or lysine amine group on the polypeptide, and so can be a thiol-reactive group such as a maleimide or a dibromomaleimide, or as defined herein, or an amine-reactive group such as an active ester (e.g., perfluorophenyl ester or tetrafluorophenyl ester), or as defined herein. Stated in another manner, the connection between Y and L or X can be the product of a reaction between cysteine, thiol or lysine amine group on the polypeptide and a thiol-reactive group such as a maleimide or dibromomaleimide on the L or X group, or an amine-reactive group on the L or X group.

In some embodiments, the X or L group is covalently bound to a terminal end of an amino acid residue or a glycan on the autoantigen Y that is not typically sialylated, i.e., the sialic acid residue is heterologous to the amino acid residue or glycan moiety. As used herein, the term “heterologous” refers to a component of a composition that is non-native to the composition, i.e., not typically found in nature in association with the rest of the entity to which it is being compared. For example, the sialic acids may be covalently bound to a glycan structure such as G0, G1, G2, G0F, G1F, or G2F. In some embodiments, the sialic acid is covalently bound to structure that is typically sialylated in a glycan such as G1S, G2S, G2S2, G1FS, G2FS, and G2FS2. In some embodiments, the sialic acid is covalently bound to a native glycan, N- or O-linked, present in the corresponding unengineered autoantigen lacking the sialic acid modification. In other such embodiments, the sialic acid is covalently bound to a novel N-linked glycan site. In other embodiments, the sialic acid is covalently bound directly to an amino acid of the autoantigen, e.g., a random lysine or cysteine, an engineered or endogenous transglutaminase site, an engineered or endogenous sortase site, an engineered Catalent formylglycine aldehyde site using formylglycine-generating enzyme (FGE), N-terminus-selective conjugation to biotherapeutics containing an N-terminal 2-hydroxyethylamine (Serine) moiety (SeriMab technology), or a novel O-linked glycan site. Any approach for determining the sites of sialylation or Siglec ligand conjugation on a biotherapeutic, including, e.g., proteolyzed product LC/MS (peptide mapping LC/MS), and LC/MS of larger product fragments (e.g., antibody Fc vs light chain, Fd′), may be used to determine the placement of Siglec ligand within the autoantigen.

In some embodiments, the X or L is covalently bound to a native element, e.g., glycan, of the autoantigen Y.

As discussed above, in some aspects of the disclosure, an engineered autoantigen is provided, wherein the engineered autoantigen (referred to hereafter as the “hypoimmunogenic autoantigen”, “modified autoantigen” or simply “subject autoantigen”) is an autoantigen that has been engineered to comprise an altered Siglec ligand profile.

In some embodiments, the altered Siglec ligand profile may include an enrichment for sialic acid relative to the parental autoantigen. Put another way, the engineered autoantigen is enriched for sialic acid, i.e., it is “hypersialylated”. For example, the engineered autoantigen may comprise one or more sialic acid moieties, e.g. 1, 2, 3, 4 or more sialic acid moieties, in some cases 5, 6, 7, 8, 9, or 10 or more sialic acid moieties, in some such instances, 11, 12, 13, 14, 15, 16, 17, 18, 19, or 20 or more moieties, whereas the parental autoantigen comprises no sialic acid moieties As another example, the subject autoantigen may comprise two or more sialic acid moieties, e.g., 2, 3, 4 or more sialic acid moieties, in some cases 5, 6, 7, 8, 9, or 10 or more sialic acid moieties, in some such instances, 11, 12, 13, 14, 15, 16, 17, 18, 19, or 20 or more sialic acid moieties, whereas the parental autoantigen comprises only one sialic acid moiety. In some embodiments, the autoantigen comprises 2-fold sialic acid or more than a corresponding unengineered autoantigen that would induce an immune reaction in the individual, for example, 3-fold more, 4-fold more, 5-fold more, 6-fold more, 7-fold more, 8-fold more, 9-fold more 10-fold more, 11-fold more, 12-fold more, 13-fold more, 14-fold more, 15-fold more, 16-fold more, 17-fold more, 18-fold more, 19-fold more, or even 20-fold more sialic acid than the unengineered autoantigen. In some embodiments, 50% or more of the glycan moieties of the engineered autoantigen, e.g., 60%, 70%, 80%, 85%, 90%, 95%, 98% or 100% of the glycan moieties, comprise a sialic acid.

In some embodiments, 50% or more of the engineered autoantigen in a sample is hypersialylated, e.g., 60%, 70%, 80%, 85%, 90%, 95%, 98% or 100% of the engineered autoantigen in a sample is hypersialylated. For example, 50% or more of the engineered autoantigen in a sample can be hypersialylated to the same extent or greater, which as described above includes embodiments where the engineered autoantigen comprises more sialic acid than a corresponding unengineered autoantigen that would induce an immune reaction in the individual. In some embodiments, 60%, 70%, 80%, 85%, 90%, 95%, 98% or 100% of the engineered autoantigen in a sample is hypersialylated to the same extent or greater.

Any approach for measuring the sialylation, i.e. the sialic acid content, of an autoantigen composition, including, e.g., glycoprotein LC/MS, Glycan LC/MS, protein LC/MS, intact drug LC/MS, capillary gel electrophoresis glycan analysis, analytical ion exchange HPLC, analytical reverse phase HPLC, analytical hydrophobic interaction chromatography HPLC, analytical mixed mode chromatography HPLC, total sialic acid or Siglec ligand analysis by plate-based assay, UV/Vis absorbance spectroscopy, surface plasmon resonance-based Siglec ligand quantitation assay, biolayer interferometry-based Siglec ligand quantitation assay, etc. may be used to determine the amount of Siglec ligand appended to an autoantigen.

In some embodiments, the non-naturally occurring Siglec ligand comprises a naturally occurring sialic acid and a non-naturally occurring linker. In some embodiments, the non-naturally occurring Siglec ligand consists essentially of a naturally occurring sialic acid and a non-naturally occurring linker. In some embodiments, the non-naturally occurring Siglec ligand comprises a non-naturally occurring sialic acid. In some embodiments, the non-naturally occurring Siglec ligand comprises a non-naturally occurring linker. In some embodiments, the non-naturally occurring Siglec ligand consists essentially of a non-naturally occurring sialic acid and a non-naturally occurring linker.

### Autoantigen Fused to CD22-Binding Polypeptide

In some embodiments, the Siglec ligand comprises a Siglec binding fragment from a Siglec-specific antibody, e.g. the CDR, the Fab, the Fab′, the Fv, the nanobody, etc. from, e.g., a monoclonal antibody, an scFv, a minibody, a diabody, a triabody, a tetrabody, a darpin, a camelid nanobody, an affimer, a fynomer, a bispecific antibody, a trispecific antibody, or the like that is specific for a Siglec. In some embodiments, the Siglec ligand comprises a Siglec binding fragment from a Siglec specific chimeric antigen receptor (“CAR”). In some embodiments, the Siglec-specific antibody or Siglec-specific CAR is specific for Siglec-2 (aka CD22). In some such embodiments, the Siglec-2 specific antibody is selected from the group consisting of epratuzumab, inotuzumab, suciraslimab, bectumomab, pinatuzumab, GTB-1550, hLL2, RFB4, JNJ-75348780, HB-22.7, m971, H10-2-4, and moxetumomab. In some such embodiments the Siglec ligand comprises a Siglec binding fragment derived from an scFv polypeptide sequence designed from epratuzumab, or a peptide selected from the group consisting of PV1 (GYINPRNDYTEYNQ; SEQ ID NO:1), PV2 (CGYRNPRNDYREYCNQ; SEQ ID NO:2), and PV3 (RNDYTE; SEQ ID NO:3), the chemical structures for which may be found in Table 2 (Kim, B. et al. Nanoscale 2020, 12, 11672-11683). In certain such embodiments, the Siglec binding fragment consists essentially of an scFv polypeptide sequence designed from epratuzumab or a peptide selected from the group consisting of PV1 (GYINPRNDYTEYNQ; SEQ ID NO:1), PV2 (CGYRNPRNDYREYCNQ; SEQ ID NO:2), and PV3 (RNDYTE; SEQ ID NO:3).

TABLE 2
Chemical structures for PV1 (SEQ ID NO: 1), PV2 (SEQ ID NO: 2), and
PV3 (SEQ ID NO: 3).

| | PV1 | PV2 | PV3 |
|---|---|---|---|
| Chemical Structure | 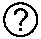 | 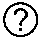 | 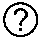 |

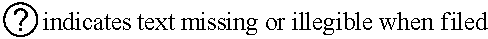

In some such embodiments, the Siglec ligand is a synthetic derived from monoclonal antibody polypeptides that include HB-22.5, 22.7, 22.23, 22.33, 22.13, and HB22.196, as described in Pearson, et al. (International Journal of Peptide Research and Therapeutics, 14, 3, 237-246 (2008)). One such peptide is “Peptide 5”, derived from the CDR2 region of monoclonal antibody HB22-7, with amino acid sequence CLGIIWGDGRTDYNSALKSRC (SEQ ID NO:4) and a disulfide bond between the N- and C-terminal cysteines.

In some embodiments, the Siglec ligand comprises a Siglec binding fragment from a Siglec-specific aptamer. Nonlimiting examples of Siglec-specific aptamers that comprise a Siglec binding fragment that finds use in the subject autoantigen include TD-05, TD-05.1, and TD-05.17.

In some such embodiments, the Siglec ligand comprises a synthetic, non-antibody-derived Siglec binding peptide, where the peptide binds with measurable affinity and high specificity to CD22. For example, peptides may be those described in WO2014044793, e.g., “Peptide 26”, otherwise known as “G635BVI071M1TK” with amino acid sequence

(SEQ ID NO: 5)
RALLSIFGSLDHRHHHRTCNTTHYRVTTMSHPQFEKKKKLRMKMSHPQ
LINTTHYRGGPTMGGSPSRQV”.

Accordingly, in some embodiments, the subject engineered autoantigen comprises an autoantigen conjugated to one or more naturally occurring Siglec ligands, i.e., a moiety comprising a naturally occurring sialic acid and a naturally occurring glycan, wherein the sialic acid and glycan are typically found in nature in association with one another to form a Siglec ligand. In other embodiments, the subject engineered autoantigen comprises an autoantigen conjugated to one or more non-naturally occurring Siglec ligands, e.g. a moiety comprising a naturally occurring sialic acid and a linker, a moiety comprising a non-naturally occurring sialic acid and a glycan found in nature as part of a Siglec ligand, a moiety comprising a non-naturally occurring sialic acid and a linker, a moiety comprising a peptide having an affinity for a Siglec, and the like. For example, in some embodiments of the engineered autoantigen, the Siglec ligand is a non-naturally occurring Siglec ligand.

In certain aspects, the engineered autoantigen may be administered to a subject in need thereof in the form of a nucleic acid encoding a fusion protein comprising the autoantigen and a CD22-binding polypeptide. The nucleic acid may be administered using a viral vector, e.g., rAAV virions that are configured to infect certain cell types and deliver the nucleic acid into the cells, where the nucleic acid is transcribed and translated to produce the engineered autoantigen. Such embodiments are also applicable to a hypoimmunogenic biotherapeutic which is a fusion protein comprising a therapeutic polypeptide and a CD22 binding polypeptide.

“AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), avian AAV, bovine AAV, canine AAV, equine AAV, primate AAV, non-primate AAV, and ovine AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc.

An “rAAV vector” as used herein refers to an AAV vector comprising a polynucleotide sequence not of AAV origin (i.e., a polynucleotide heterologous to AAV), typically a sequence of interest for the genetic transformation of a cell, such as, a nucleic acid encoding an engineered autoantigen or a hypoimmunogenic biotherapeutic described herein. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome, such as a nucleic acid encoding an engineered autoantigen or a hypoimmunogenic biotherapeutic to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

### Methods of Manufacture

As discussed above, the engineered autoantigens are autoantigens which have been modified to comprise heterologous Siglec ligands (be they heterologous to the autoantigen or heterologous to the amino acid to which they are appended) and/or elevated amounts of Siglec ligand(s) that naturally occur on said autoantigens. Typically, the modification is not simply by associating the Siglec ligand with the autoantigen via a formulation, e.g., a liposomal formulation. Rather, the modification is a covalent binding of Siglec ligand to the autoantigen.

Any convenient methods of covalently binding sialic acids to autoantigen may be used, any of which may be deployed to modify an autoantigen of choice to become an engineered autoantigen of the present disclosure. For example, the modification may be performed by biosynthesis. By “biosynthesis”, it is meant a synthesis process that is mediated by cells. For example, in the Golgi apparatus, a subset of the 20 known sialyltransferases attach sialic acids to underlying monosaccharides such as galactose via three different types of linkage (α2,3, α2,6, and α2,8). By engineered biosynthesis, it is meant a synthesis process that is mediated by cells that have been engineered to perform the process, in some instances de novo, in other instances, in a modified way. Thus, for example, a producer cell line may be genetically engineering to express one or more sialyl transferases, e.g. sialyltransferase (EC 2.4.99), beta-galactosamide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8); lactosylceramide alpha-2,3-sialyltransferase (EC 2.4.99.9), or other enzymes in an enzymatic pathway, e.g. CMP-Neu5Ac hydroxylase, sialate-4-O-acetyl transferase, sialate-4-O-acetylesterase, sialate-7(9)-O-acetyltransferase, sialate-8-O-methyl transferase, sialate-9-)-acetyltransferase, etc. that drives the covalent binding of a specific sialic acid to the autoantigen or that targets specific novel amino acid residues for covalent modification with sialic acid. As another example, a producer cell line could be fed a precursor substrate that will be incorporated by the producer line into the manufactured autoantigen as a specific Siglec ligand. Any producer cell that finds use in the expression of proteins for use as an autoantigen may be used in this process, for example a mammalian cell (CHO, HEK, etc.), an insect cell (SF9, etc.), a bacterium, a protozoan (Leishmania, etc.). as disclosed in, e.g., WO2017093291, WO2019002512, WO2019234021, the full disclosures of which are incorporated herein in their entirety by reference.

As another example, the modification may be performed by chemical conjugation. By “chemical conjugation”, it is meant a process that occurs exogenous to a cell. Thus, for example, the Siglec ligand might be enzymatically or chemically linked to the autoantigen after biosynthesis from producer cell line. Nonlimiting examples of such in vitro processes are disclosed in U.S. Pat. Nos. 7,220,555 and 6,376,475B, and U.S. Pat. No. 5,409,817, the full disclosures of which are incorporated herein by reference. In some such embodiments, a linker may be deployed to covalently link the sialic acid to the autoantigen. Many examples of linkers exist in the art, any of which may be used to chemically conjugate sialic acid(s) to the autoantigen to arrive at engineered autoantigens of the present disclosure.

As a third example, specifically directed to embodiments in which the Siglec ligand is a peptide or polypeptide sequence, e.g., an scFv or peptide derived from epratuzumab, e.g., PV1, PV2 or PV3, the modification may be performed by genetic engineering of the autoantigen to include a fusion of the peptide/polypeptide sequence with the autoantigen. For example, the polynucleotide used to produce the autoantigen may be modified by standard molecular biology cloning techniques to include a polynucleotide sequence encoding the peptide/polypeptide in the same translational reading frame (“In frame”), such that upon transcription and translation of the autoantigen in a producing cell, the autoantigen is fused to the peptide/polypeptide sequence covalently. The peptide/polypeptide sequence will be genetically engineered into a domain of the autoantigen that does not form the epitope recognized by autoimmune antibodies against the autoantigen.

In certain aspects, the engineered autoantigen that includes a fusion of the autoantigen to a siglec ligand (SigL), such as, a polypeptide that binds to a siglec may be included in a pharmaceutical composition as a nucleic acid encoding the fusion protein comprising the autoantigen and the SigL. In certain aspects, the pharmaceutical composition may include a recombinant adenoassociated virus (rAAV) that includes a payload that is a nucleic acid encoding the fusion protein comprising the autoantigen and the SigL. The rAAV may include a capsid that targets the rAAV to a particular type of cell, such as, kidney or liver cells.

In certain aspects, a rAAV that includes a payload that is a nucleic acid encoding a fusion protein comprising a biotherapeutic and a SigL, e.g., an anti-CD22 antibody, a peptide that binds to CD22, and the like is also contemplated. Nonlimiting examples of biotherapeutics include protein therapeutics, e.g., antibody therapeutics, fusion protein therapeutics, enzyme therapeutics; viral therapeutics; cell therapeutics; and nucleic acid therapeutics.

The present invention further provides host cells, e.g., isolated (genetically modified) host cells, comprising a subject nucleic acid. A subject host cell can be an isolated cell, e.g., a cell in in vitro culture. A subject host cell is useful for producing a subject rAAV virion, as described herein. Where a subject host cell is used to produce a subject rAAV virion, it is referred to as a “packaging cell.” In some embodiments, a subject host cell is stably genetically modified with a subject nucleic acid. In other embodiments, a subject host cell is transiently genetically modified with a subject nucleic acid.

A subject nucleic acid is introduced stably or transiently into a host cell, using established techniques, including, but not limited to, electroporation, calcium phosphate precipitation, liposome-mediated transfection, and the like. For stable transformation, a subject nucleic acid will generally further include a selectable marker, e.g., any of several well-known selectable markers such as neomycin resistance, and the like.

A subject host cell is generated by introducing a subject nucleic acid into any of a variety of cells, e.g., mammalian cells, including, e.g., murine cells, and primate cells (e.g., human cells). Suitable mammalian cells include, but are not limited to, primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. A subject host cell can also be made using a baculovirus to infect insect cells such as Sf9 cells, which produce AAV (see, e.g., U.S. Pat. No. 7,271,002; U.S. patent application Ser. No. 12/297,958).

In some embodiments, a subject genetically modified host cell includes, in addition to a nucleic acid comprising a nucleotide sequence encoding a variant AAV capsid protein, as described above, a nucleic acid that comprises a nucleotide sequence encoding one or more AAV rep proteins. In other embodiments, a subject host cell further comprises an rAAV vector. An rAAV virion can be generated using a subject host cell. Methods of generating an rAAV virion are described in, e.g., U.S. Patent Publication No. 2005/0053922 and U.S. Patent Publication No. 2009/0202490.

### Methods of Use

In some aspects of the invention, methods are provided for the treatment of individuals having an undesired immune response to an antigen, e.g., pathogenic immune response.

In some aspects of the invention, methods are provided for the treatment of an individual having an autoimmune disease where the individual produces an immune response to an autoantigen, where the method comprises administering to the individual an engineered autoantigen to reduce the immune response to the antigen.

In some aspects of the invention, methods are provided for the treatment of an individual having an immune response to a biotherapeutic.

The terms “treatment”, “treating” and the like are used herein to generally mean obtaining a desired pharmacologic and/or physiologic effect. The effect may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The therapeutic agent may be administered during or after the onset of disease. The treatment of ongoing disease, where the treatment stabilizes or reduces the undesirable clinical symptoms of the patient, is of particular interest.

The terms “individual,” “subject,” “host,” and “patient,” are used interchangeably herein and refer to any mammalian subject for whom diagnosis, treatment, or therapy is desired, particularly humans.

The engineered autoantigen compositions of the present disclosure find particular use in the treatment of diseases that require modulation of the immune response to be effective. Such engineered autoantigens may find use in the treatment of diseases that require repeat or chronic administration of the engineered autoantigen to be effective. There are many instances of such conditions, like autoimmune conditions, of which a few nonlimiting examples are provided below and elsewhere. It is expected that the ordinarily skilled artisan will be able to extrapolate from these examples to other indications and autoantigens as known in the art.

For example, the individual may be suffering from an autoimmune disease, e.g., Pemphigus Vulgaris, celiac disease, primary biliary cholangitis, membranous nephropathy, rheumatoid arthritis, Systemic lupus erythematosus, psoriatic arthritis, ankylosing spondylitis, Crohn's disease, ulcerative colitis, psoriasis, hidradenitis suppurativa, uveitis, or juvenile idiopathic arthritis.

In particular, the individual may be suffering from pemphigus vulgaris. In such instances, the method may comprise administering to the individual an engineered DSG-1 and/or DSG-3 in an amount effective to treat the pemphigus vulgaris.

In particular, the individual may be suffering from Celiac disease. In such instances, the method may comprise administering to the individual an engineered Gliadin and/or type 2 transglutaminase in an amount effective to treat the Celiac disease.

As another example, the individual may be suffering from primary biliary cholangitis. In such cases, the method may comprise administering to the individual an engineered PDC-E2 in an amount effective to treat the primary biliary cholangitis.

As another example, the individual may be suffering from membranous nephropathy. In such cases, the method may comprise administering to the individual an engineered PLA2R in an amount effective to treat the membranous nephropathy.

As another example, the individual may be suffering from arthritis, e.g., rheumatoid arthritis. In such instances, the method may comprise administering to the individual an engineered autoantigen citrullinated protein or peptide, carbamylated protein or peptide, or acetylated protein or peptide in an amount effective to treat the arthritis.

As another example, the individual may be suffering from Systemic lupus erythematosus. In such instances, the method may comprise administering to the individual an engineered autoantigen, such as double-stranded DNA (dsDNA) or ribonucleoproteins (e.g., nucleosomes or the the Sm antigens of the U-1 small nuclear ribonucleoprotein complex) in an amount effective to treat the Systemic lupus erythematosus.

The engineered autoantigen may be administered to a subject having an autoimmune disease for a period of 1 month or more, where the engineered autoantigen is administered daily, weekly, bi-weekly, or any frequency in between. The engineered autoantigen may be administered to a subject having an autoimmune disease for a period of 3 months or more, 6 months or more, 1 year or more, where the engineered autoantigen is administered daily, weekly, bi-weekly, monthly, quarterly or semi-annually, or any frequency in between. The frequency and time period may be adjusted based on the subject's response. For example, the frequency and/or time period of treatment may be increased if the subject has relatively high titers of autoantibodies against the autoantigen and the frequency and/or time period of treatment may be decreased if the subject has relatively low titers of autoantibodies against the autoantigen.

### Pharmaceutical Compositions

In methods of treating an individual with the subject engineered autoantigen, the patient will typically be administered a pharmaceutical composition comprising the subject engineered autoantigen. By a pharmaceutical composition, it is meant an engineered autoantigen of the present disclosure that has been formulated with a pharmaceutically acceptable carrier. As used herein, a “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The pharmaceutical compositions of the disclosure are administered at a therapeutically effective dosage, e.g., a dosage sufficient to provide treatment for the disease states previously described. Administration of the compounds of the disclosure or the pharmaceutically acceptable salts thereof can be via any of the accepted modes of administration for agents that serve similar utilities. While human dosage levels have yet to be optimized for the compounds of the disclosure, these can be readily extrapolated from doses administered to a relevant animal model, e.g., mice that results in treatment of the disease or disorder in that animal model. Generally, an individual human dose is from about 0.01 to 2.0 mg/kg of body weight, preferably about 0.1 to 1.5 mg/kg of body weight, and most preferably about 0.3 to 1.0 mg/kg of body weight. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered will, of course, be dependent on any or all of the following: the subject and disease state being treated, the severity of the affliction, the manner and schedule of administration and the judgment of the prescribing physician. It will also be appreciated that amounts administered will depend upon the molecular weight of the autoantigen, the amount of Siglec ligand covalently bound, and the size of the linker.

While all typical routes of administration are contemplated (e.g., oral, topical, transdermal, injection (intramuscular, intravenous, or intra-arterial)), it is presently preferred to provide liquid dosage forms suitable for injection. Generally, depending on the intended mode of administration, the pharmaceutically acceptable composition will contain about 0.1% to 95%, preferably about 0.5% to 50%, by weight of the subject engineered autoantigen of the disclosure, the remainder being suitable pharmaceutical excipients, carriers, etc. Dosage forms or compositions containing active ingredient in the range of 0.005% to 95% with the balance made up from non-toxic carrier can be prepared.

The subject pharmaceutical compositions can be administered either alone or in combination with other pharmaceutical agents. These compositions can include other medicinal agents, pharmaceutical agents, carriers, and the like, including, but not limited to other active agents that can act as immune-modulating agents and more specifically can have inhibitory effects on B-cells, including anti-folates, immune suppressants, cyostatics, mitotic inhibitors, and anti-metabolites, or combinations thereof.

Liquid pharmaceutically administrable compositions can, for example, be prepared by dissolving, dispersing, etc. an active composition of the disclosure (e.g., a lyophilized powder) and optional pharmaceutical adjuvants in a carrier, such as, for example, water (water for injection), saline, aqueous dextrose, glycerol, glycols, ethanol or the like (excluding galactoses), to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands. Actual methods of preparing such dosage forms are known, or will be apparent, to those skilled in this art; for example, see Remington: The Science and Practice of Pharmacy, Pharmaceutical Press, 22nd Edition, 2012. The composition or formulation to be administered will, in any event, contain a quantity of the active compound in an amount effective to treat the symptoms of the subject being treated.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

All synthetic chemistry was performed in standard laboratory glassware unless indicated otherwise in the examples. Commercial reagents were used as received. Microwave reactions were performed in an Anton Paar Monowave 400 using the instrument software to control heating time and pressure. Analytical LC/MS was performed either on a Waters Acquity UPLC Instrument with PDA and Single Quadrupole Detector (with alternating positive and negative ion scans) using Masslynx Software or a Shimadzu LCMS-2020 using LabSolutions software. Retention times were determined from the extracted 214 and/or 254 nm UV chromatogram. Prep HPLC was performed either on a Waters Autopurification System consisting of Fraction module 2767, Pump 2545 and 2998 PDA detector using Masslynx software/Agilent 1260 Infinity Autopurification system with DAD detector or on a Gilson system using a 215 liquid handler, 333 and 334 pumps, UV/VIS-155 detector, and Trilution Ic software. ¹H NMR was performed either on a Bruker Avance 400 MHz or a Bruker Fourier 300 MHz using Topspin software. Analytical thin layer chromatography was performed on silica (Sigma Aldrich TLC Silica gel 60 F₂₅₄ aluminum or glass TLC plate, silica gel coated with flourescent indicator F254) and is visualized under UV light. Silica gel chromatography was performed manually, or with Teledyne ISCO CombiFlash NextGen 300⁺ automated chromatography for gradient elution.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1: Concept for Class of CD22-Engaging Recombinant Autoantigen Derivatives that Treat Patients for Autoantigen-Driven Diseases by Eliminating Autoantigen-Specific Pathogenic B Cells while Leaving Non-Pathogenic B Cells Unaffected (Non-Tolerized)

Pathogenic autoantibodies are driving factors for a number of autoimmune diseases, including rheumatoid arthritis, systemic lupus erythematosus, membranous nephropathy, IgA nephropathy, pemphigus vulgaris, Graves' disease, and myasthenia gravis. The B cell and its clonotypic B cell receptor (BCR) sit at the heart of autoantibody generation, being responsible for the selection and secretion of autoantibody from pathogenic, autoantigen-specific B cell clones. A novel class of biomolecules with suppressing, anergizing, and/or deleting activity for pathogenic, autoantigen-specific B cells has been designed. The B cell-clone-targeted biologics function based on the principal that specific B cell clones can be inhibited through Siglec inhibitory receptor recruitment to clonotypic anti-drug BCRs. The Siglecs are a class of immunoinhibitory, sialic acid-binding lectin proteins, expressed on most or all types of hematopoietic cells. Such deletion of autoantigen-specific B cells effects disease suppression and/or reversion.

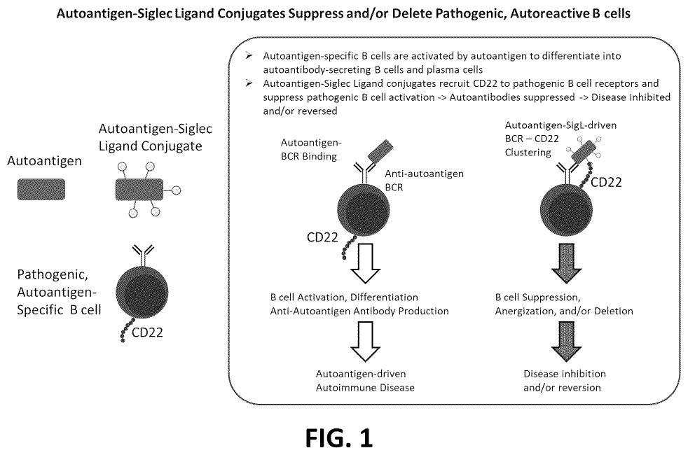

FIG. 1 depicts one aspect of the model for CD22-engaging recombinant autoantigen derivatives that suppress, anergize, and/or delete pathogenic, autoantigen-specific B cells. Clonotypic BCR—Siglec-2 co-engagers (including Autoantigen-Siglec Ligand conjugates and Autoantigen-CD22 binder fusions) suppress, silence, and/or delete autoantigen-specific, pathogenic B cells by virtue of the physical recruitment of the inhibitory CD22 receptor to the BCR complex after autoantigen interaction with the BCR on a clonotypic, autogenic-specific B cell. Such deletion of autoantigen-specific B cells effects disease suppression and/or reversion.

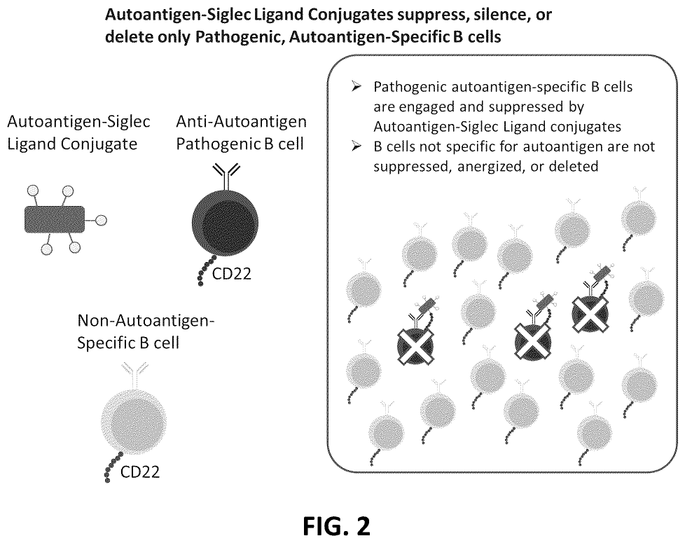

FIG. 2 depicts another aspect of the model for CD22-engaging autoantigen derivatives that suppress, anergize, and/or delete pathogenic, autoantigen-specific B cells. Clonotypic BCR—Siglec-2 co-engagers (including Autoantigen-Siglec Ligand conjugates and Autoantigen-CD22 binder fusions) act only on autoantigen-specific B cells (suppression, silencing, and/or deletion) without exerting such effects on non-autoantigen-specific B cells and thus leaving the non-autoantigen-specific portion of the B cell receptor intact.

### Example 2: Different Formats for Siglec-2/CD22-Engaging, Tolerizing Autoantigen Derivatives

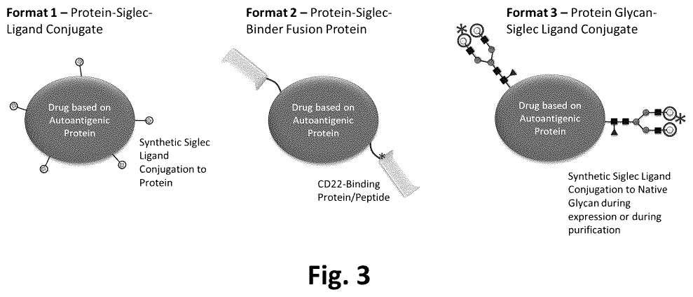

Three formats of engineered, Siglec-2/CD22-engaging, autoantigen-tolerizing biologics are proposed. One representative structure of each format is shown in FIG. 3. The proteinaceous component of each biologic is based on disease-driving autoantigens (e.g., PLA2R for Membranous Nephropathy, Dsg1 and Dsg3 for Pemphigus Vulgaris, MuSK for Myasthenia Gravis, gliadin peptides for celiac disease). The autoantigen is engineered for CD22-binding activity using the 3 presented formats. Formats 1 and 3 use chemically, enzymatically, or biosynthetically-incorporated, synthetic, small-molecule Siglec ligands to impart Siglec-2 binding activity to the given molecule.

“Format 1” is a protein covalently modified on its polypeptide chains with one or more conjugatable Siglec ligand-linker structures. Conjugation of the Siglec-2 ligand-linker structure can be achieved through site-specific or non-site-specific methodologies.

“Format 2” for engineering of a pathogenic B cell-targeting biologic uses protein- or peptide-based CD22 binder incorporation into the polypeptide chain of an autoantigen-based protein. Examples of such CD22 binders would include: 1) immunoglobulin-based binders, such as Fab domains, single-chain Fv (scFv) fragments, diabodies, and single-domain antibody fragments (camelid V_{H}H or shark V_{NAR}), 2) non-immunoglobulin-based binding domains, such as affibodies, fynomers, monobodies, DARPins, Knottins, Variable Lymphocyte Receptors (VLRs), and affimers, 3) CD22-binding peptides, such as peptide aptamers, and 4) oligonucleotide-based Siglec binders, such as oligonucleotide aptamers.

“Format 3” is a biologic modified on a natural or engineered glycan with Siglec ligand structures, where Siglec-2 ligand incorporation occurs either biosynthetically during drug expression in cells or via chemical and/or chemoenzymatic conjugation after purification of the biologic. Format 3 includes approaches where a Siglec-2 ligand-based substrate would be fed to cells during drug expression to enable biosynthetic incorporation in drug glycans. Incorporation of Siglec ligand into glycan could also be achieved through treatment with Siglec-2 ligand-based enzyme substrate in an in vitro protein translation system.

All three of the illustrated formats would enable CD22 recruitment to clonotypic B cells during B cell receptor binding, with consequent suppression of B cell activation, proliferation, and differentiation, ultimately driving autoantigen-specific B cell suppression, anergization, and/or deletion, blockage of autoantibody production, and consequent tolerization to the disease-driving autoantigen.

### Example 3: Production and Characterization of Protein-Siglec Ligand Conjugates

### Purpose

To produce Siglec ligand-linker conjugates of proteins for evaluation in in vivo immunogenicity and tolerization experiments. Proteins were either produced in-house or procured from commercial sources, as described below.

### Materials and Methods

Antibody (adalimumab anti-hTNFα hIgG1) Expression, Purification, and Analytics

For antibody expression, the ExpiFectamine 293 Transfection kit (Life Technologies, A14524) was used to transfect suspension Expi293F cells (Life Technologies, A14527) with Heavy Chain and Light Chain plasmids (pTT5-based) at a 1:1 ratio. Media was harvested 3-6 days post-transfection by centrifugation and filtered using 0.2 μm PES vacuum sterile single-use filter unit (ThermoScientific, 5670020).

Purification was performed with 1.5 mL MabSelect Sure resin (Cytiva/GE Cat #: 17-5438-03) for each 250 mL culture supernatant. Briefly, each column was equilibrated with PBS pH 7.2 and loaded with culture supernatant. After the loading step, the column was washed with PBS pH 7.2 and eluted with 10 mL IgG Elution buffer (Thermo Scientific Ref 21004). The pH of the elution pool was adjusted with 1 mL 1 M Sodium Phosphate pH 6.5 for each 10 mL elution pool. Finally, buffer exchange was performed with PBS pH 7.2 using a 30 kDa Amicon Ultra-15 Centrifugal Filter Unit.

Analysis of endotoxin content was performed using the Charles River Endosafe PTS 0.01-1 EU/ml detection. Size exclusion chromatography was performed on an Agilent Chemstation HPLC-SEC with a Sepax-Zenix SEC-300, 200 mm×7.8 mm ID, 3 uM column. Capillary gel electrophoresis (cGE) was performed on a Caliper LabChip GXII Protein 200 with the Perkin Elmer Chip (Cat #760499). LC-MS analysis was performed on SciEX LC 5600+, ExionLC AD, Analyst TF 1.8.1 with an Agilent AdvanceBio Desalting-RP, Column 1000A, 10 um.

### Protein-Siglec Ligand Conjugation

Pentafluorophenyl (PFP) conjugatable Siglec Ligand linker was added to reaction mixtures at a molar ratio of 4-30 times above protein based on desired degree of labeling in the presence of 10% v/v of 50 mM Sodium Tetraborate pH 8.5 and 10% v/v DMSO. Reactions were incubated for 3 hours at 25° C. After the 3 h incubation period, 10% v/v of 1 M Tris-HCl pH 8.0 was added to quench the unreacted linker-payload. Neutralized reactions were then allowed to incubate at 25° C. for 15 min.

### Protein Siglec-Ligand Conjugate Purification

Quenched conjugation reactions are purified by preparative size exclusion chromatography at 4° C. using either Superdex 200 Increase 10/300 GL or HiLoad 16/600 Superdex 200 pg at a flow rate of 0.75 mL/min, with PBS pH 7.2.

### Analysis of Protein-Siglec Ligand Conjugates

### LC-MS

Mass spectrometer: SciEX LC 5600⁺, ExionLC AD, Analyst TF 1.8.1
HPLC: Agilent AdvanceBio Desalting-RP, Column 1000A, 10 um
2.1 mm×12.5 mm, flow rate 400 ul/min, Sample load: 5 ug
Buffer A: Water+0.1% Formic Acid
Buffer B: Acetonitrile+0.1% Formic Acid

### Analytical SEC

Superose 6 Increase 10/300 GL
Injection Volume: 25 uL at 1 mg/mL of sample.
Buffer: 50 mM Sodium Phosphate+400 mM Sodium Perchlorate, pH 6.2
Flow rate: 0.7 mL/min
Run time 40 min at 25° C.

### Capillary Gel Electrophoresis

Caliper LabChip GXII Protein 200

### Endotoxin Measurement

Charles River Endosafe PTS Cartridge 0.01-1 EU/ml
Lonza Pyrogene C Endpoint Endotoxin Assay

### Example 4: Prophylactic Tolerization of Mice to Adalimumab Antigen with Siglec Ligand-Adalimumab Conjugate Pre-Dosing

### Purpose

The purpose of this experiment was to test for prophylactic tolerization using Siglec-2 ligand conjugates with recombinant antigen. Mice were evaluated for tolerization to adalimumab after pre-dosing with adalimumab-Siglec-2 Ligand conjugates. Parental adalimumab hIgG1 is highly immunogenic in mice, with a strong immunoglobulin response after subcutaneous dosing at 4 mg/kg.

### Materials and Methods

Adalimumab hIgG1 and Adalimumab-Siglec Ligand conjugates were prepared as described in Example 3.

### Tolerization and Anti-Drug Antibody Production in C57BL6 Mice

To evaluate the production of antibodies specific to adalimumab, two control groups of C57BL6 mice were immunized with adalimumab or the individual SigL-conjugated adalimumab antibodies. Conjugates bear BPC-Neu5Gc-based Siglec Ligand-Linker structures with affinity for Siglec-2. On study day −1, animals were randomized into treatment groups based on body weight. On study day 0, animals were bled for baseline serum and then injected subcutaneously (s.q.) with adalimumab or the individual SigL-conjugated adalimumab antibodies once every seven days for a total of four injections over twenty-eight days. The individual antigens were prepared by making a 0.8 mg/ml antigen solution in sterile buffered saline. Animals were then injected with 0.1 ml (˜4 ml/kg) of the 0.8 mg/ml antigen on the rear flanks. The total dose based on a 20 g mouse would be 4 mg/kg per injection. Animals were bled via the retro-orbital sinus weekly throughout the study under inhaled isoflurane anesthesia. Whole blood was collected into Microvette EDTA capillary collection tube (Sarstedt Inc) and then further processed following manufacturer's instructions to collect serum. Samples were then stored at −80C until analysis was performed.

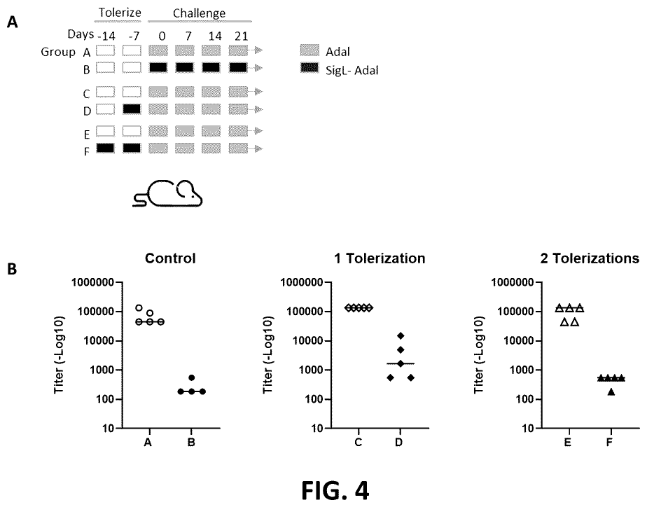

To evaluate if dosing with SigL-conjugated adalimumab induces tolerization to adalimumab, mice were primed with either a single s.q. dose of SigL-conjugated adalimumab (4 ml/kg), two s.q. doses of SigL-conjugated adalimumab (4 ml/kg) given seven days apart or control injections of PBS. Seven days after the last injection, animals were challenged with unconjugated adalimumab as described above. Briefly, animals were injected s.q. with adalimumab once every seven days for a total of four injections over twenty-eight days. FIG. 4A depicts a schematic of the dosing schedule. Animals were bled via the retro-orbital sinus weekly throughout the study under inhaled isoflurane anesthesia. Whole blood was collected into Microvette EDTA capillary collection tube (Sarstedt Inc.) and then further processed following manufacturer's instructions to collect serum. Samples where then stored at −80C until analysis was performed.

Anti-drug antibody (ADA) response assays were performed on 96-well assay plates (Nunc Plates, Black 96-Well Immuno Plates, Thermo Scientific, 437111) coated with antigen, as follows. A mixture of adalimumab and adalimumab conjugates was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

FIG. 4 depicts the evaluation of anti-drug antibody responses in mice for adalimumab and Siglec Ligand-adalimumab conjugates, with or without pre-dosing with Siglec Ligand-adalimumab conjugate or vehicle. Adalimumab-Siglec ligand conjugate is non-immunogenic (Group B) while adalimumab is highly immunogenic, driving a strong anti-drug antibody response (Group A). Comparison of groups C (vehicle) and D (1 adalimumab-SigL pre-dose) shows that pre-dosing mice with a single 4 mg/kg dose of adalimumab-Siglec ligand conjugate is sufficient to cause a strong suppression of ADA response to 4 subsequent subcutaneous doses with adalimumab. Groups E (vehicle) and F (2 adalimumab-SigL pre-doses) demonstrate that pre-dosing with two doses of adalimumab-Siglec Ligand drives an even stronger suppression of adalimumab ADA response to 4 subsequent subcutaneous doses with adalimumab. Thus, this study demonstrates that Siglec Ligand-conjugated drug exerts a strong, suppressive effect on antibody response to immunization with antigen, and shows immunological memory to exposure to Siglec Ligand-coengaging antigen.

### Example 5: Suppression of Immunogenicity Increase in the Presence of Pre-Existing Immunogenicity

### Purpose

The purpose of this experiment was to test for suppression of increased immunogenicity (to parental antigen) in the face of pre-existing antigen immunogenicity, using Siglec-2 ligand conjugates with recombinant antigen. Mice were evaluated for immunogenicity to adalimumab and/or adalimumab-SigL after pre-dosing with unmodified adalimumab.

### Materials and Methods

Adalimumab hIgG1 and Adalimumab-Siglec Ligand conjugates were prepared as described in Example 3.

### Anti-Antigen Antibody Production in C57BL6 Mice

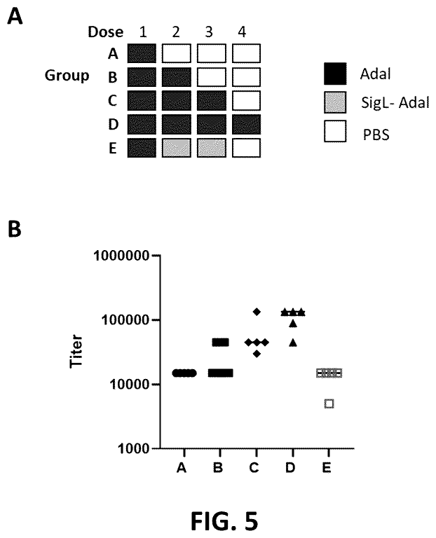

To evaluate the production of antibodies specific to adalimumab-SigL conjugates after pre-dosing with adalimumab, C57BL6 mice were immunized with adalimumab one, two, three, or four times (groups A to D), or once with adalimumab followed by two doses of adalimumab-SigL (Group E) (FIG. 5). Conjugates bear BPC-Neu5Gc-based Siglec Ligand-Linker structures with affinity for Siglec-2. On study day −1, animals were randomized into treatment groups based on body weight. On study day 0, animals were bled for baseline serum and then injected subcutaneously (s.q.) with adalimumab, SigL-conjugated adalimumab antibody, or PBS, once every seven days for a total of four injections over twenty-eight days. The individual antigens were prepared by making a 0.8 mg/ml antigen solution in sterile buffered saline. Animals were then injected with 0.1 ml (˜4 ml/kg) of the 0.8 mg/ml antigen on the rear flanks. The total dose based on a 20 g mouse would be 4 mg/kg per injection. Animals were bled via the retro-orbital sinus weekly throughout the study under inhaled isoflurane anesthesia. Whole blood was collected into Microvette EDTA capillary collection tube (Sarstedt Inc) and then further processed following manufacturer's instructions to collect serum. Samples were then stored at −80C until analysis was performed. FIG. 5A depicts a schematic of the dosing schedule.

Anti-drug antibody (ADA) response assays were performed on 96-well assay plates (Nunc Plates, Black 96-Well Immuno Plates, Thermo Scientific, 437111) coated with antigen, as follows. A mixture of adalimumab and adalimumab conjugates was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. Titers were determined by the dilution of serum that gives a 2× OD above background.

### Results and Conclusions

FIG. 5 depicts the evaluation of anti-drug antibody responses in mice for adalimumab and Siglec Ligand-adalimumab conjugates, where mice were first immunized with adalimumab prior to subsequent adalimumab or adalimumab-SigL dosing. All groups (A to E, FIG. 5A) received a single 4 mg/kg starting dose of adalimumab, following by three weekly doses of adalimumab, adalimumab-SigL, and/or PBS vehicle. From one to four doses of adalimumab (groups A to D), mice show progressively stronger anti-adalimumab titers (FIG. 5B). In contrast, mice in group E that received a single adalimumab dose followed by two doses of adalimumab-SigL show no greater titer than mice (group A) dosed once with adalimumab and two doses of vehicle (PBS). Thus, this study demonstrates that Siglec Ligand-conjugated immunogen is no more immunogenic than vehicle, even where animals are pre-dosed with the parental immunogen. “Adal” is adalimumab. “SigL-Adal” is adalimumab-CD22 ligand conjugate. Adalimumab-CD22 ligand conjugate bears BPC-Neu5Gc-based Siglec Ligand-Linker structures. Serum IgG levels against adalimumab/adalimumab conjugate were measured 27 days after the first adalimumab dose. FIG. 5, panel B shows individual animal serum IgG titer values (n=5 mice).

### Example 6: Concept for Class of Protein-Based CD22-Engaging Autoantigens that Suppress Existing, Autoantigen-Directed, B Cell-Driven Autoimmunity

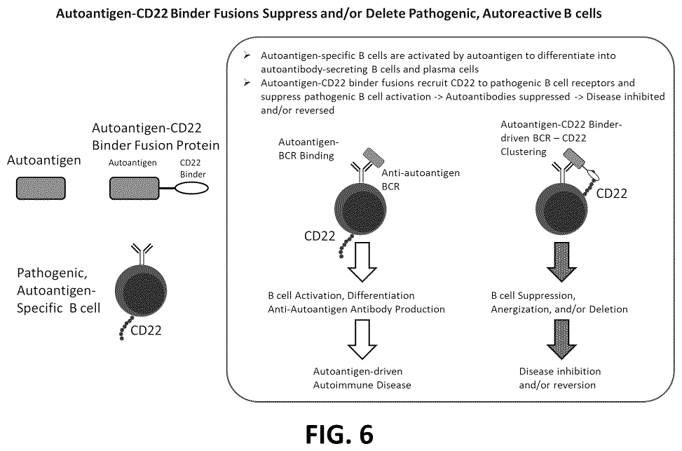

FIG. 6 depicts an aspect of a model for protein-based CD22-coengaging autoantigens that suppress and/or reverse autoantigen-specific, B cell-driven autoimmune disease: protein-based B cell receptor—CD22 co-engagers suppress or silence autoantigen-specific B cell activation by virtue of the physical recruitment of the inhibitory CD22 receptor to autoantigen-specific B cell receptor complexes after autoantigen interaction with the BCR on clonotypic, autogenic-specific B cells. Such deletion of autoantigen-specific B cells effects disease suppression and/or reversion.

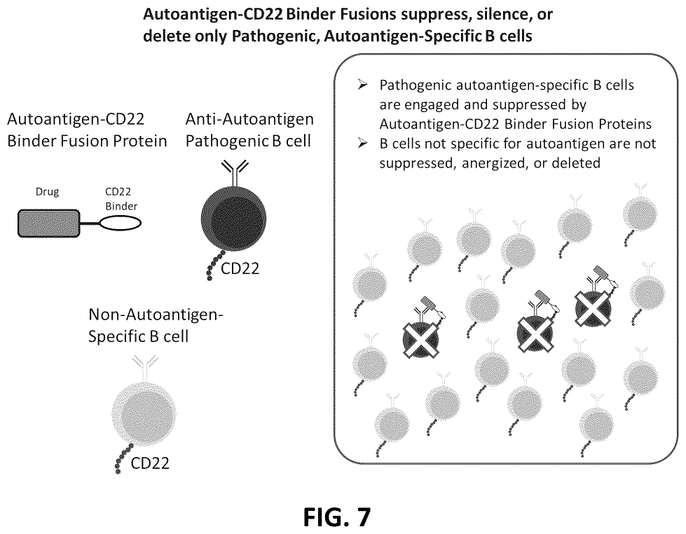

FIG. 7 depicts another aspect of the model for recombinant CD22-engaging autoantigens with suppressed anti-drug antibody responses: protein-based B cell receptor—CD22 co-engagers suppress, silence, and/or delete only autoantigen-specific B cells while leaving intact the portion of the B cell repertoire that is not specific for autoantigen.

### Example 7: Suppression of B Cell Receptor-Mediated Activation of Mouse Primary B Cells by Beads Co-Presenting Anti-IgD Antibody and Anti-CD22 Antibody

### Purpose and Introduction

The purpose of this experiment was to test for the ability of CD22 co-engagement with BCR using protein-based CD22 ligands to suppress B cell activation.

The platform technology described rests on the premise that activation of B cells through their clonotypic B cell receptor can be suppressed through physical recruitment of the CD22/Siglec-2 inhibitory coreceptor to co-engaged B cell receptor. CD22 recruited to the B cell receptor is phosphorylated on its ITIM cytoplasmic motif tyrosines by virtue of its proximity to the high local protein kinase activity at the B cell receptor. Phosphorylated CD22 then recruits phosphatases, such as SHP-1 and SHP-2, to the cell surface, in proximity of the B cell activation complex. Such elevated local phosphatase activity dephosphorylates components of the B cell activation complex necessary for B cell activation, thus shutting down responses to B cell receptor engagement. Under normal circumstances, the Siglec-2 immunoinhibitory mechanism acts as a check on aberrant B cell activation, safeguarding against autoreactive antibody production, hyperinflammation, and autoimmunity. The described platform technology exploits this natural phenomenon to cloak autoantigenic or foreign proteins as self, dampening B cell activation only on B cell clones that are specific for the given autoantigen or foreign protein and thus blocking immunoglobulin production against the autoantigen or foreign protein, while leaving B cell responses to other antigens intact.

The high diversity of primary B cell populations, and high diversity of B cell receptor sequences and clones (as high as 10¹² per human), presents a challenge for studying BCR agonism in vitro with a single, well-defined BCR antigen. For this reason, pan-BCR activators, such as anti-IgD or anti-IgM antibodies, that can bind, crosslink, and activate the BCR—regardless of B cell/BCR clonality—are used to evaluate BCR activation in vitro. Thus, in the context of recombinant autoantigens that engage CD22, the anti-IgD/IgM antibody acts as a model for a patient autoantigen. In the experiments described in this example, an anti-mouse IgD monoclonal antibody is coated on beads on its own or in combination with a mouse CD22 antibody to study the effects of Siglec-2-B cell receptor co-engagement on B cell activation with a protein-based CD22 ligand.

### Materials & Methods—In Vitro Murine B-Cell Activation Assay

In Vitro Murine B-Cell Activation with Anti-IgD/Anti-CD22 Beads

To generate antibody coated beads, anti-biotin microbeads (Miltenyi Biotech) were conjugated to the following biotinylated antibodies following manufacturer's protocols: biotinylated-anti-mouse-IgM (Southern Biotech), biotinylated anti-CD22 (Southern Biotech), biotinylated-rat-IgG1 isotype (Southern Biotech), or biotinylated-rat-IgG2a isotype (Southern Biotech).

To induce B cell activation, splenocytes from C57BL6 mice were harvested into single cell suspension, subjected to red cell lysis using ACK buffer, and plated at a concentration of 200,000 cells per well in round bottom 96 well plates in complete RPMI media. Cells were stimulated for 20 hours by the addition of the different antibody coated bead to cell ratio of 1:1. B cell activation was then assessed by flow cytometry.

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1× phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on a ZE5 cytometer (BioRad). Data analysis was performed using FlowJo (v10.8.0) software.

### Results and Conclusions

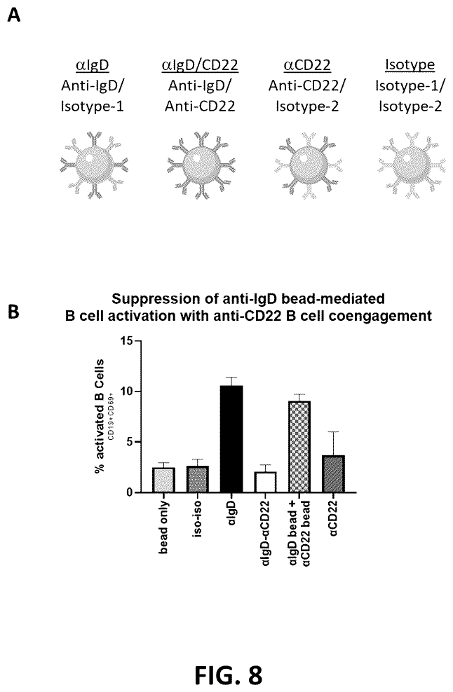

FIG. 8 depicts the in vitro B cell activation assay where mouse primary B cells were treated with beads coated with different combinations of B cell receptor agonizing anti-IgD antibody, anti-CD22 antibody, and/or isotype controls. The treatment groups, as shown in FIG. 8A, were: 1) beads alone, 2) isotype antibody-coated beads, 3) anti-IgD beads, 4) beads coated with both anti-IgD and anti-CD22, 5) anti-CD22 beads, and 6) pooled beads coated separately with anti-IgD and anti-CD22. Splenocytes from C57BL6 mice were incubated with coated beads and compared for activation, as measured by CD69 upregulation. CD69 levels on the different treatment groups were evaluated through the percentage of cells that are CD69-positive (FIG. 8B).

Where beads coated only with anti-IgD (FIG. 8B, black bar) trigger B cell activation, beads co-immobilized with anti-IgD and anti-CD22 antibodies show a complete lack of activation (FIG. 8B, white bar). Importantly, the suppressive effect of anti-CD22 must occur in cis and not in trans, as a mixture of beads individually coated with anti-IgM and anti-CD22 (FIG. 8B, hatched bar) does not suppress the agonist activity of anti-IgM beads. As expected, beads coated only with anti-CD22 (FIG. 8B, right gray bar) do not cause B cell activation. In summary, this experiment demonstrates that BCR—CD22 co-engagement by a protein-based CD22 binder is sufficient to block B cell activation where an agonist (anti-BCR) is otherwise present. Moreover, a protein-based CD22 binder can be used to suppress B cell activation through the B cell receptor. This result supports the concept of an autoantigen-CD22 binder construct where a set of autoantigen-specific B cell clones are engaged by their clonotypic B cell receptors and subsequently suppressed, anergized, and/or deleted as a consequence of CD22 recruitment to the B cell receptor by the protein-based CD22 binder.

### Example 8: Production and Characterization of Antibody-CD22 Binder Fusion Proteins

### Purpose

To produce anti-IgM×anti-human CD22 scFv fusion protein and anti-IgM antibody Siglec ligand-linker conjugates of proteins for evaluation in in vitro B cell signaling assays and in vivo immunogenicity experiments. Proteins were produced in-house as described below.

### Materials and Methods

Antibody (Anti-IgM Human IgG1 Chimeric Antibody, Anti-IgM×Anti-CD22 scFv IgG1 Chimeric Bispecific Antibody) Expression, Purification, and Analytics

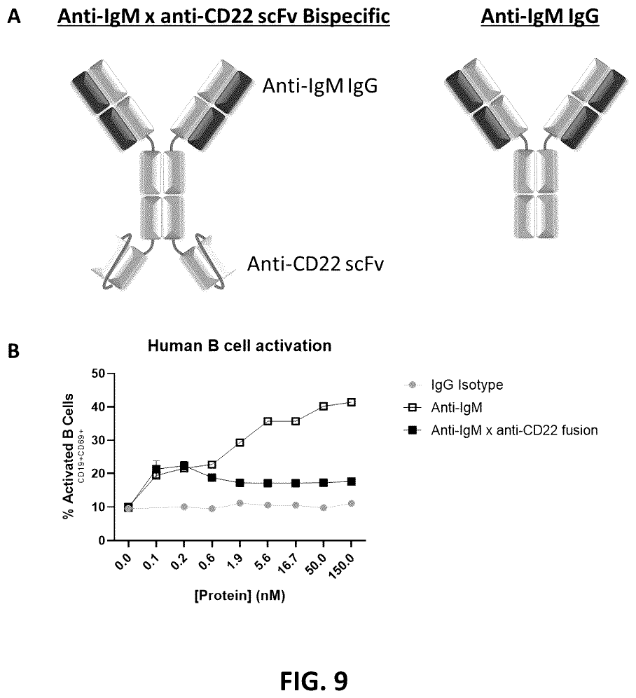

Schematics of the bispecific anti-IgM×anti-CD22 antibody and its parental IgM antibody are shown in FIG. 9A.

For antibody expression, the ExpiFectamine 293 Transfection kit (Life Technologies, A14524) was used to transfect suspension Expi293F cells (Life Technologies, A14527) with Heavy Chain and Light Chain plasmids (pTT5-based) at a 1:1 ratio. Media was harvested 3-6 days post-transfection by centrifugation and filtered using 0.2 μm PES vacuum sterile single-use filter unit (ThermoScientific, 5670020).

Purification was performed with MabSelect Sure resin (Cytiva/GE Cat #: 17-5438-03) for each culture supernatant. Briefly, each column was equilibrated with PBS pH 7.2 and loaded with culture supernatant. After the loading step, the column was washed with PBS pH 7.2 and eluted with 10 mL IgG Elution buffer (Thermo Scientific Ref 21004). The pH of the elution pool was adjusted with 1 mL 1 M Sodium Phosphate pH 6.5 for each 10 mL elution pool. Finally, buffer exchange was performed with PBS pH 7.2 using a 30 kDa Amicon Ultra-15 Centrifugal Filter Unit.

Analysis of endotoxin content was performed using the Charles River Endosafe PTS 0.01-1 EU/ml detection. Size exclusion chromatography was performed on an Agilent Chemstation HPLC-SEC with a Sepax-Zenix SEC-300, 200 mm×7.8 mm ID, 3 uM column. LC-MS analysis was performed on SciEX LC 5600⁺, ExionLC AD, Analyst TF 1.8.1 with an Agilent AdvanceBio Desalting-RP, Column 1000A, 10 um.

### Results

Bispecific anti-IgM×anti-CD22 and anti-IgM IgG were purified to homogeneity with >99% purity by analytical size exclusion chromatography, correct identity and high purity by SDS-PAGE, accurate heavy and light chain masses by LC/MS, and endotoxin levels less than 2.5 EU/mg. Final preps were evaluated for CD22 binding activity in Example 9, and for splenocyte B cell activation/suppression and binding activity in Example 10.

### Example 9: Binding Analysis of Antibody-CD22 Binder Fusion Proteins with Siglec-2/CD22 by Surface Plasmon Resonance

### Purpose

To determine the in vitro binding properties of Antibody-human CD22 Binder Fusion Protein for recombinant human CD22 ectodomain.

### Materials and Methods

Binding assays for synthetic CD22 ligand binding to CD22 receptor ectodomain were run on a Biacore 8K+ instrument (Cytiva). Surface preparation for CD22 conjugates binding to human CD22 protein consisted of two steps, covalent immobilization of streptavidin followed by capture of Avi-tagged and biotinylated human CD22 His-tag Avi-tag protein (R&D Systems, Cat #AVI10191. Using standard amine coupling protocols, Streptavidin (Invitrogen, Cat #: 434301) was immobilized to a Cytiva CM5 chip (Cytiva, Cat #BR100530) by injecting at 100 ug/mL in Sodium Acetate, pH 4.5 (Cytiva, Cat #BR100350) on both flow cells, yielding a final response of 2500 RU. Capture of biotinylated human CD22-Fc was performed on channels 1-8. 2 ug/ml Human CD22 was injected on the active flow cell (2) for 60 seconds at 10 μL/min with a running buffer of 10 mM HEPES, 150 mM NaCl, 0.05% T20, pH7.5, yielding ˜50 RU of captured CD22.

Binding experiments of Siglec-ligand conjugated proteins were performed on the surface prepared above in HBS-EP+ (10 mM HEPES, 150 mM NaCl, 0.05% Tween-20, pH 7.5) as the running buffer. Conjugates were serially diluted 1:1 in running buffer from 1 μM to 31.25 nM and injected over both the reference and active flow cells for 120 seconds at 30 μL/min. The conjugates were then allowed to dissociate from the surface for 240 seconds. Surfaces were regenerated with 30 section injections of 3M MgCl₂ at 30 ul/min.

### Results and Conclusions

Sensorgrams fit well to a 1:1 interaction model. The dissociation constant for anti-IgM×anti-CD22 scFv bispecific fusion interaction with the human CD22 ectodomain was measured at 3.0 uM from an association rate of 3.95×10⁵ M⁻¹s⁻¹ and a dissociation rate of 1.16 s⁻¹.

### Example 10: Suppression of B Cell Receptor-Mediated Activation of Human Primary B Cells by a Fusion Bispecific Binding Both Surface IgM and CD22

### Purpose

The purpose of the experiment described here is to evaluate a protein-based BCR×CD22 co-engager for suppressive activity on human B cell receptor-mediated B cell activation. This example follows on from the experiment described in example 8, using pan-B cell receptor agonism in a pool of B cell clones to study the effects of CD22-B cell receptor co-engagement on B cell activation. In this example, an engineered bispecific antibody with dual binding activity for membrane IgM (BCR) and CD22 was produced (Example 8) and compared for BCR agonist activity with the parental anti-IgM antibody. As in example 7, an anti-B cell receptor antibody, this time against human IgM, functions as a surrogate for recombinant autoantigen, where a recombinant autoantigen-CD22 binder construct would engage the clonotypic B cell receptors on autoantigen-specific B cells.

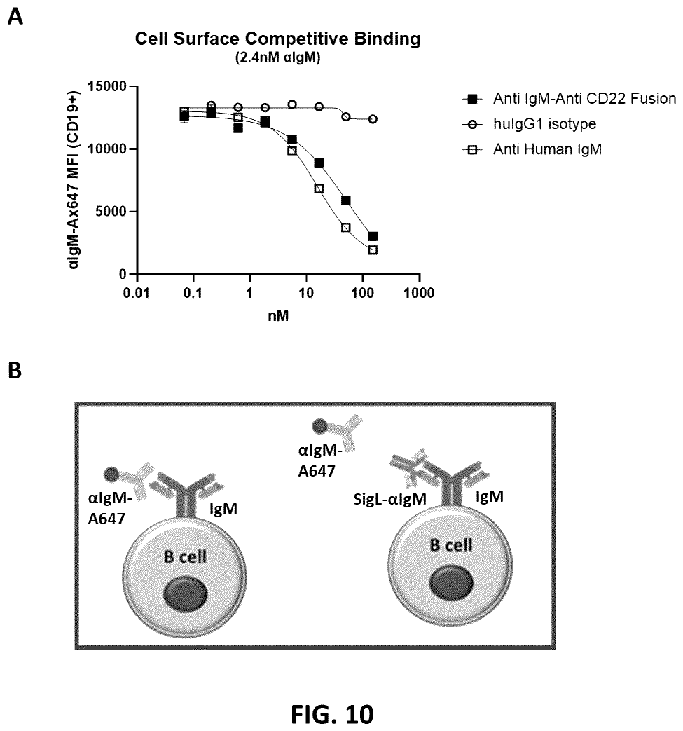

To control for impacts of scFv addition to the C-terminus of the anti-IgM heavy chain on BCR binding potency, a competition binding assay was used to compare the binding activities of the parental anti-IgM and the bispecific anti-IgM×anti-CD22 bispecific (FIG. 10). By eliminating the possibility of differences in IgM binding activity between the test articles, it was ensured that suppressive effects on B cell activation were due to Siglec-2× BCR co-engagement and not a general damaging of anti-IgM for BCR engagement.

### Materials and Methods

Anti-IgM human IgG1 chimeric antibody and anti-IgM×anti-CD22 scFv IgG1 chimeric bispecific antibody were prepared as described in Example 8.

### In Vitro Human B-Cell Activation and Surface IgM Competition Assays

Human PBMCs (StemExpress) were plated at a concentration of 200,000 cells per well in round bottom 96 well plates in complete RPMI media. Cells were stimulated for 18 hours by the addition of increasing concentration of anti-human IgM or anti-human IgM×anti-CD22 bispecific antibodies. B cell activation was then assessed by flow cytometry.

Competition of anti-human IgM binding was carried out on human PBMCs from the same donors used in assays described above. For this assay cells were seeded at a concentration of 200,000 cells per well in round bottom 96 well plates in complete RPMI media. Cells were then incubated with human Fc-block (BD Biosciences) for five minutes. Following this incubation period, anti-human-IgM-AlexaFluor647 at a fixed concentration of 2.4 nM was added to the cells along with RPMI alone or an increasing titration of anti-CD19 and either non-fluorescently labeled anti-human IgM or non-fluorescently labeled anti-human IgM×anti-CD22 bispecific antibodies. Cells were incubated at 4C for 30 minutes in the dark. Afterwards cells were washed twice by centrifugation with staining buffer (1% bovine serum albumin/0.1% sodium azide/1× phosphate buffered saline) and antibody binding was analyzed by flow cytometry (ZE5, BioRad) by determination of the mean fluorescence intensity (MFI) of at least 5,000 cells.

### Flow Cytometry and Analysis

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1× phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on ZE5 (BioRad). Data analysis was performed using FlowJo (v10.8.0) software.

### Results and Conclusions

FIG. 9B depicts an in vitro B cell activation assay, using human primary B cells and either a B cell receptor agonizing anti-IgM antibody or the anti-IgM×anti-CD22 bispecific described in examples 8 and 9. Anti-IgM shows strong, dose-dependent activation of human primary B cells, as measured by upregulation of cell-surface CD69. In contrast, anti-IgM×anti-CD22 bispecific is strongly suppressed for B cell activation, showing that a BCR-CD22 co-engager using a protein-based CD22 binder is sufficient to effect B cell receptor suppression.

A BCR competition assay was used to control for perturbations in bispecific antibody binding to the B cell receptor (FIG. 10). The test articles are identical to those used in the B cell activation experiment in FIG. 9. The anti-IgM×anti-CD22 bispecific and parental anti-IgM antibody show equivalent competition for the binding of a labeled anti-IgM antibody, thus demonstrating a lack of perturbation in BCR binding for Siglec Ligand conjugates. Importantly, this result demonstrates that the suppressive activity of the bispecific antibody is due to its CD22 binding activity and not due to trivial perturbations in IgM binding activity.

In summary, a protein-based binder of CD22 can be used to design a B cell receptor—CD22 coengager with B cell suppressive activity, including in cases where the B cell receptor engager is an autoantigen targeting autoantigen-specific B cells.

### Example 11: Prophylactic Tolerization of Mice to Adalimumab Antigen Through Siglec Ligand-Adalimumab Conjugate Pre-Dosing: Long-Term Analysis of Mouse Responses

### Purpose

The purpose of this experiment was to test for an active, tolerizing activity of adalimumab-Siglec Ligand conjugate against subsequent exposure to parental, immunogenic adalimumab. This example is a continuation of the experiment described in Example 4, with analysis of serum anti-drug titers at time points later than 28 days, out past day 100.

### Materials and Methods

Example 3 describes the materials and methods for the production of adalimumab and adalimumab-Siglec Ligand (Ligand-to-Drug Ratio, LDR, 6.3) test articles. The conjugate bears BPC-Neu5Gc-based Siglec Ligand-Linker structures with affinity for Siglec-2. Example 4 describes the initial analysis of conjugate-mediated tolerization.

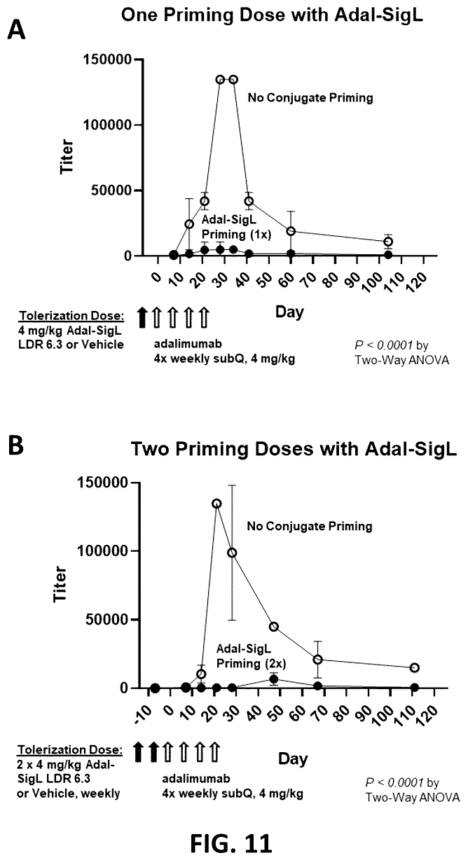

In summary, as described in Example 4, to evaluate if dosing with SigL-conjugated adalimumab induces tolerization to adalimumab, female C57Bl/6 mice (n=5 per group) were primed with either a single subcutaneous dose of SigL-conjugated adalimumab (4 mg/kg), two subcutaneous doses of SigL-conjugated adalimumab (4 mg/kg) given seven days apart, or one or two control injections of PBS. The individual antigens were prepared by making a 0.8 mg/ml antigen solution in sterile buffered saline. Animals were then injected with 0.1 ml (˜4 mg/kg) of the 0.8 mg/ml antigen on the rear flanks. The total dose based on a 20 g mouse would be 4 mg/kg per injection. Seven days after the last adalimumab conjugate injection, animals were challenged four times with unconjugated adalimumab as described in Example 4: animals were injected subcutaneously with adalimumab once every seven days for a total of four injections over twenty-eight days. FIG. 4, panel A depicts a schematic of the dosing schedule. Blood samples were collected over a period of more than 100 days (see FIG. 11). Animals were bled via the retro-orbital sinus throughout the study under inhaled isoflurane anesthesia. Whole blood was collected into Microvette EDTA capillary collection tube (Sarstedt Inc.) and then further processed following manufacturer's instructions to collect serum. Samples were then stored at −80C until analysis was performed.

Anti-drug antibody (ADA) response assays were performed on later time points exactly as described for earlier time points in Example 4.

### Results and Discussion

FIG. 11 depicts the evaluation of anti-drug antibody responses in mice for adalimumab and Siglec Ligand-adalimumab conjugates, with or without pre-dosing with Siglec Ligand-adalimumab conjugate or vehicle, over the duration of several months. Comparison of groups pre-dosed with vehicle vs those administered one (FIG. 11, panel A) or two (FIG. 11, panel B) pre-doses of adalimumab-SigL shows that pre-dosing mice with a single 4 mg/kg dose of adalimumab-Siglec ligand conjugate is sufficient to cause a strong suppression of ADA response to 4 subsequent subcutaneous doses with adalimumab. Analysis of the later time points sampled in this example demonstrates that such antigen tolerization is highly durable. No signs of ADA response or recovery were seen in tolerized animals even past day 100. Thus, this study demonstrates that Siglec Ligand-conjugated drug exerts not only a strong, but also stable, suppressive effect on antibody response to immunization with antigen, and shows immunological memory to exposure to Siglec Ligand-coengaging antigen. In addition, these results show that the B cell suppression mechanism of the protein-Siglec Ligand is an active and dominant mechanism that leads to specific functional deletion of antigen-specific B cells that drive the production of anti-antigen antibodies.

### Example 12: Evaluation of the Temporal Durability of Adalimumab-Siglec Ligand-Induced Tolerization of Mice to Subsequent Dosing with Standard Adalimumab Antibody

### Purpose

Having established the tolerogenic activity of immunogen-Siglec Ligand conjugates in Examples 4, 5, and 11, the goal of this study was to evaluate the duration of the tolerized state in mice administered adalimumab-Siglec Ligand conjugate. In Example 11, mice were dosed with immunogenic adalimumab during weeks immediately subsequent to pre-dosing with adalimumab-Siglec Ligand conjugate. In this example, mice were dosed several times with immunogenic adalimumab at much later time points, out to day 202 after the first dose of tolerogen.

### Materials and Methods

To evaluate the durability of the induced tolerization status established with the SigL-conjugated adalimumab (as exemplified in Examples 4 and 11), female C57Bl/6 mice (n=5 per group) were primed with four subcutaneous doses of either SigL-conjugated adalimumab (4 mg/kg) or unconjugated adalimumab (4 mg/kg) given seven days apart over a 28 day period. Animals in both groups were later challenged with single subcutaneous doses of unconjugated adalimumab (4 mg/kg) at days 91, 147, 174, and 202. Animals were injected with 0.1 ml (˜4 mg/kg) of the 0.8 mg/ml test article preparations on the rear flanks. The total dose based on a 20 g mouse would be 4 mg/kg per injection per dose. Bleeds were taken for anti-drug antibody analysis at days 0, 28, 112, 146, 168, 201, and 232. Anti-drug antibody (ADA) response assays were performed as described in Example 4.

### Results and Discussion

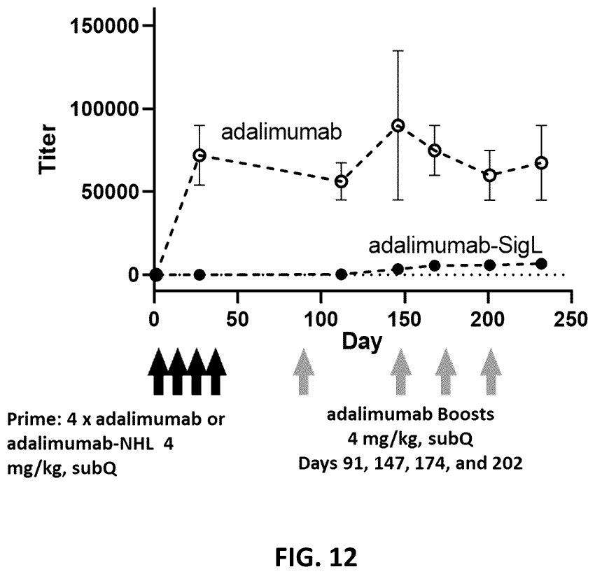

FIG. 12 depicts tolerization of mice to subsequent dosing with immunogenic adalimumab antibody. Mice were administered either 4 weekly doses of 4 mg/kg adalimumab-Siglec Ligand conjugate (solid circles) or 4 mg/kg adalimumab (open circles), followed by 4 doses of 4 mg/kg adalimumab at much later time points than in Example 11: days 91, 147, 174, and 202. As in the experiments depicted in FIG. 4 and FIG. 11, animals pre-treated with adalimumab-Siglec Ligand show a strong suppression of anti-drug antibody, while adalimumab pre-dosed animals establish a strong, saturating level of ADA. Despite repeated administration with immunogenic adalimumab, mice pre-dosed with adalimumab-Siglec Ligand conjugate never show the strong ADA levels seen in animals only dosed with adalimumab. In addition, mice dosed with adalimumab at much later time points continue to show resistance to adalimumab immunogenicity, demonstrating a strong and durable tolerization induced by the Siglec Ligand conjugate. The mice are specifically tolerized to subsequent dose with adalimumab immunogen even as late as day 202. The study was terminated before any signs of loss of tolerance could be detected, so the duration of tolerance may well be much longer than 200 days. As in Example 11, these results also show that the B cell suppression mechanism of the protein-Siglec Ligand is an active and dominant mechanism that leads to specific functional deletion of antigen-specific B cells that drive the production of anti-antigen antibodies.

### Example 13: Analysis of Antigen-Specific B Cells in Mice Administered Adalimumab and Adalimumab-Siglec Ligand

### Purpose

The purpose of this set of experiments was to evaluate the antigen-specific B cell responses to immunogenic adalimumab and hypoimmunogenic adalimumab-Siglec Ligand. Analysis of antigen-specific B cell responses is an orthogonal means of study of immunogen responses with respect to serum anti-drug antibody analysis and can be used to independently corroborate the ADA tolerization results described in the above examples. To study antigen-specific B cells and tolerization mechanisms, ex vivo cytometric analysis of splenic B cell populations was performed after sequences of immunogen and/or tolerogen administration.

### Materials and Methods

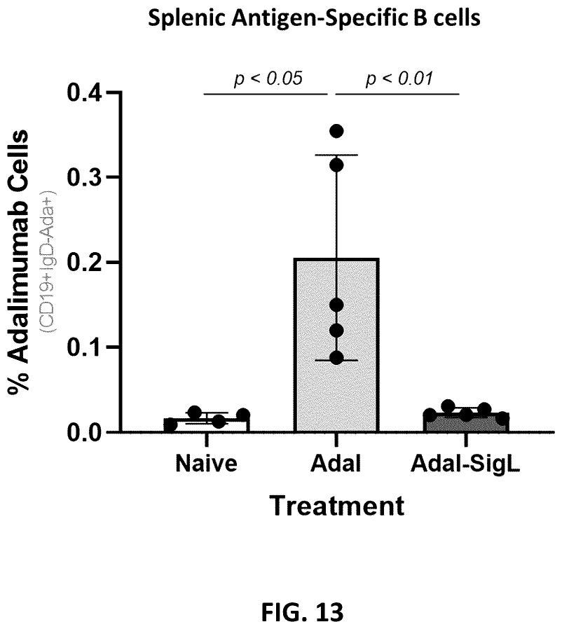

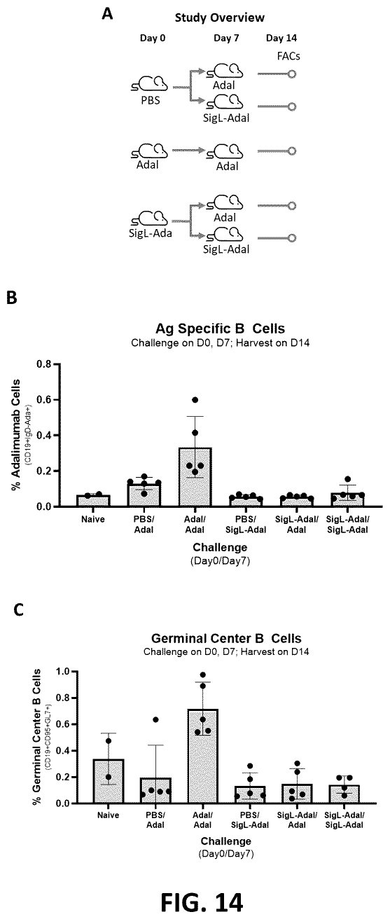

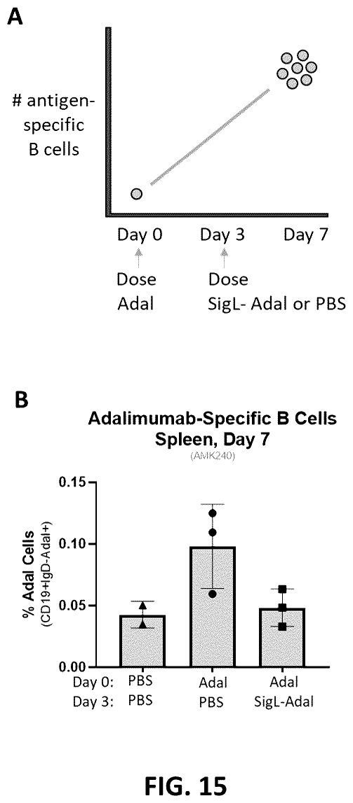

The production of adalimumab and adalimumab-Siglec Ligand (Ligand-to-Drug Ratio, LDR, 6.3) test articles was as described in Example 3. Conjugates used in these experiments bear BPC-Neu5Gc-based Siglec Ligand-Linker structures with affinity for Siglec-2. Ligand-to-Drug (LDR) ratios for the conjugates were as follows: 1) FIG. 13, LDR 6.4; 2) FIG. 14, LDR 8.4; 3) FIG. 15, LDR 9.2.

Flow cytometry and Analysis

To measure B cell activation following the described stimulation, cells were washed twice by spinning cells at 1200 rpm for 5 minutes and rinsing with PBS. Cells were then resuspended in staining buffer (1% bovine serum albumin/0.1% sodium azide/1× phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were then incubated in the dark for an additional 30 minutes at room temperature. Cells were then washed three times with staining buffer and then analyzed on a ZE5 Cell Analyzer (BioRad). Data analysis was performed using FlowJo (v10.8.0) software.

Ex Vivo Analysis of Adalimumab-Specific B cells

For studies evaluating the generation of adalimumab-specific B cells, C57Bl/6 mice were dosed with a single subcutaneous dose of either SigL-adalimumab (4 mg/kg) or unconjugated adalimumab (4 mg/kg). Splenocytes were then isolated from animals seven days after dosing. In the experiment depicted in FIG. 15, mice that had been dosed with unconjugated adalimumab (4 mg/kg) on Day 0 were dosed again on Day 3 with SigL-adalimumab (4 mg/kg, subcutaneous) or vehicle (PBS) prior to splenocyte isolation on Day 7 of the study.

For the evaluation of germinal center formation (FIG. 14), C57Bl/6 mice were dosed with a single subcutaneous dose of either SigL-adalimumab (4 mg/kg), unconjugated adalimumab (4 mg/kg) or vehicle (PBS) on Day 0. Animals in each arm of this study were then dosed on Day 7 with either a single subcutaneous dose of SigL-adalimumab (4 mg/kg) or unconjugated adalimumab (4 mg/kg). Formation of adalimumab-specific B cells and germinal center B cells were then analyzed on Day 14 of the study as described below.

At the specified time points described above, splenocytes were harvested from individual animals and made into single cell suspension, subjected to red cell lysis using ACK buffer, and plated at a concentration of 200,000 cells per well in round bottom 96 well plates in complete RPMI media. Cells were then processed for flow cytometry as described above. For adalimumab-specific B cells, splenocytes were stained with anti-CD4, ant-CD8a, anti-F4/80, anti-LY6G, anti-CD19, anti-IgD (BD Biosciences, Biolegend, Fisher, eBioscience) and adalimumab-alexa468 conjugate (Invitrogen). For germinal center B cells, splenocytes were stained with anti-CD45, anti-CD19, anti-GL7, and anti-CD95. Cells were then incubated in the dark for an additional 30 minutes at room temperature. Cells were then washed three times with staining buffer and then analyzed on a ZE5 Cell Analyzer (BioRad). Data analysis was performed using FlowJo (v10.8.0) software.

### Results and Discussion

FIG. 13 depicts the cytometric analysis of splenic, antigen-specific B cells from mice dosed subcutaneously with either vehicle, 4 mg/kg adalimumab, or 4 mg/kg adalimumab-Siglec Ligand LDR 6.4. Adalimumab-specific B cells (CD45⁺CD19⁺IgD⁻Adal⁺) were evaluated by flow cytometry 7 days post adalimumab or SigL-adalimumab challenge. Antigen-specific B cells were detected in the spleens in all mice dosed with adalimumab at a mean abundance of 0.2% of splenic IgD⁻ B cells. Adalimumab-specific B cells were absent in the spleens of mice dosed with SigL-adalimumab. We conclude that, where adalimumab administration drives the establishment of splenic adalimumab-specific B cells and the subsequent presence of anti-drug antibodies in blood, adalimumab-Siglec Ligand treatment does not trigger such establishment of drug-specific B cells. The tolerization results presented in the above examples indicate that adalimumab-Siglec Ligand is not only preventing expansion of adalimumab-specific B cell clones but also deleting anti-adalimumab B cells from the B cell repertoire.

FIG. 14 depicts cytometric analysis of splenic antigen-specific B cells and germinal center B cells after dosing with sequences of adalimumab, adalimumab-Siglec Ligand, and/or drug vehicle. Mice were dosed subcutaneously on either day 0 or day 7 with either vehicle (PBS), 4 mg/kg adalimumab, or 4 mg/kg adalimumab-Siglec Ligand LDR 8.4. Dosing sequences for the different animal groups are shown in FIG. 14, panel A. Adalimumab-specific B cells (CD45+CD19+IgD−Adal+) were evaluated by flow cytometry at day 14 (FIG. 14, panel B). Germinal center B cells (CD19+CD95+GL7+) were harvested on day 14 after adalimumab oradalimumab-Siglec Ligand/vehicle challenges on days 0 and 7 (FIG. 14, panel C). The data in FIG. 14 demonstrate that adalimumab dosing induces both splenic adalimumab-specific B cell clones and increase in the abundance of germinal center B cells. Dosing animals with adalimumab-Siglec Ligand at day 0 followed by adalimumab dosing at day 7 shows a complete lack of adalimumab-specific B cells (FIG. 14, panel B), corroborating the tolerizing B cell-clone specific activity of adalimumab-Siglec Ligand conjugate.

FIG. 15 depicts cytometric analysis of splenic antigen-specific B cells after dosing with sequences of adalimumab, adalimumab-Siglec Ligand, and/or drug vehicle. Mice were dosed subcutaneously on days 0 and 3 with either vehicle (PBS), 4 mg/kg adalimumab, or 4 mg/kg adalimumab-Siglec Ligand LDR 9.2 as shown in FIG. 15, panel A. Adalimumab-specific B cells (CD45+CD19+IgD−Adal+) were evaluated by flow cytometry at day 7 (FIG. 15, panel B). These data show that dosing adalimumab-Siglec Ligand after adalimumab administration is sufficient to eliminate pre-existing adalimumab-specific B cells, thus demonstrating the therapeutic potential of drug-Siglec Ligand conjugates for the elimination of antigen-specific B cells from the B cell repertoire.

### Example 14: Production and Characterization of Adalimumab and Adalimumab-Anti-Mouse CD22 scFv Fusion Protein

### Purpose

To produce adalimumab and adalimumab-anti-mouse CD22 scFv fusion protein for evaluation in in vivo immunogenicity experiments. Proteins were produced in-house as described below. The mouse CD22 scFv binder used in these studies was separately measured by surface plasmon resonance to have a dissociation constant of 18.3 nM for mouse CD22 and is thus a strong binder for CD22.

### Materials and Methods

Adalimumab Anti-TNFα Antibody Adalimumab-Anti-CD22 Fusion Protein (Anti-TNFα×Anti-CD22 scFv IgG1 Chimeric Bispecific Antibody) Expression, Purification, and Analytics

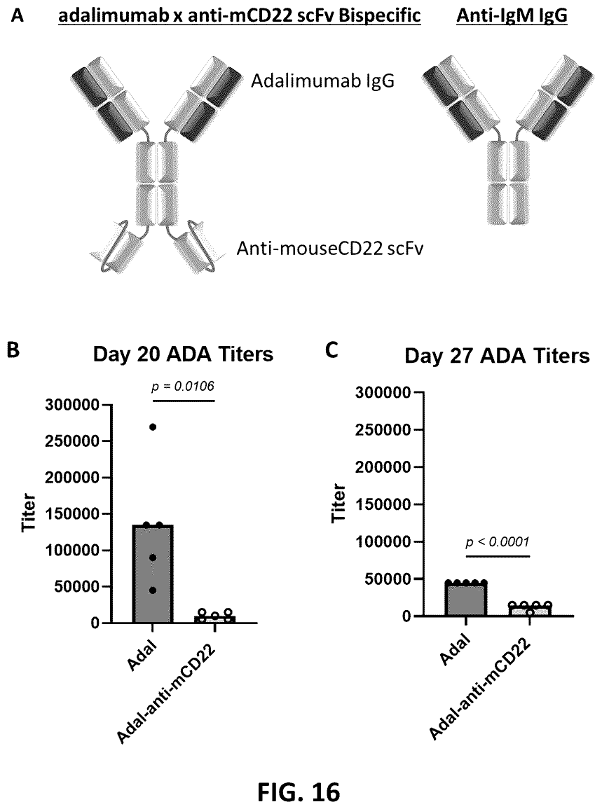

A fusion protein of the adalimumab heavy chain with an anti-mouse CD22 scFv clone (depicted in FIG. 16, panel A) was designed and cloned for expression in a mammalian expression vector. This adalimumab heavy chain-anti-mCD22 fusion construct was cotransfected with an expression vector for adalimumab light chain. Adalimumab was similarly expressed using cotransfection of separate constructs encoding the adalimumab heavy and light chains. The ExpiFectamine 293 Transfection kit (Life Technologies, A14524) was used to transfect suspension Expi293F cells (Life Technologies, A14527) with both plasmids (pTT5-based) at a 1:1 ratio. Media was harvested 3-6 days post-transfection by centrifugation and filtered using 0.2 μm PES vacuum sterile single-use filter unit (ThermoScientific, 5670020).

Purification was performed with MabSelect Sure resin (Cytiva/GE Cat #: 17-5438-03). Briefly, a MabSelect Sure column was equilibrated with PBS pH 7.2 and loaded with culture supernatant. After the loading step, the column was washed with PBS pH 7.2 and eluted with IgG Elution buffer (Thermo Scientific Ref 21004). The pH of the elution pool was adjusted with 1 M Sodium Phosphate pH 7. Finally, buffer exchange was performed with PBS pH 7.2 using a Sartorius Vivacell 100 30 kD ultrafiltration unit. MabSelect eluate was further purified by preparative size exclusion chromatography (Superdec 10/300 GL 200 Increase).

Analysis of endotoxin content was performed using the Charles River Endosafe PTS 0.01-1 EU/ml detection. Analytical size exclusion chromatography was performed on an Agilent Chemstation HPLC-SEC with a Sepax-Zenix SEC-300, 200 mm×7.8 mm ID, 3 uM column. SDS-PAGE analysis was performed on a 4-12% Bis Tris SDS gel. LC-MS analysis was performed on SciEX LC 5600+, ExionLC AD, Analyst TF 1.8.1 with an Agilent AdvanceBio Desalting-RP, Column 1000A, 10 um.

### Results

Bispecific adalimumab×anti-CD22 and adalimumab IgG were purified to homogeneity with >95% purity by analytical size exclusion chromatography, correct identity and high purity by SDS-PAGE, accurate heavy and light chain masses by LC/MS, and endotoxin levels less than 2.5 EU/mg. Final preps were evaluated for in vivo immunogenicity suppression in Example 15.

### Example 15: In Vivo Suppression of Anti-Drug Antibody in Mice Treated with an Antibody-Anti-Mouse CD22 Single Chain Fv Fusion Protein

### Purpose

The purpose of this experiment was to test for hypo-immunogenicity of adalimumab-anti-mouse CD22 scFv fusion protein relative to parental adalimumab monoclonal antibody. Parental adalimumab hIgG1 is highly immunogenic in mice, with a strong immunoglobulin response after a single 4 mg/kg dose, while the addition of CD22 binding functionality into the adalimumab antibody should enable tolerization to adalimumab. This example set out to corroborate the in vitro B cell suppressive effects shown in Examples 7 (B cell suppression with bead-coimmobilized anti-BCR and anti-CD22) and 10 (suppression of human primary B cell activation with anti-IgM-anti-human CD22 fusion protein) with in vivo assessment of effects on immunogenicity in mice for a biotherapeutic fused to a protein-based CD22 binder.

### Materials and Methods

Adalimumab and adalimumab-anti-mouse CD22 scFv fusion protein were prepared as described in Example 14.

To evaluate the production of antibodies specific to adalimumab and/or adalimumab-anti-mouse CD22 scFv fusion protein, 8 week old female C57BL/6 mice (n=5 per group) were immunized through intravenous injection with adalimumab or adalimumab-anti-mouse CD22 scFv fusion protein. On study day −1, animals were randomized into treatment groups based on body weight. On study day 0, animals were bled for baseline serum and then injected IV with adalimumab or adalimumab-anti-mouse CD22 scFv fusion protein. Animals were dosed subcutaneously with 4 mg/kg of adalimumab or adalimumab-anti-mouse CD22 scFv fusion protein. Animals were bled via the retro-orbital sinus weekly throughout the study under inhaled isoflurane anesthesia. On study day, 28 animals were anesthetized with inhaled isoflurane anesthesia and then bled via cardiac puncture and then sacrificed by cervical dislocation. Whole blood was collected into Microvette EDTA capillary collection tubes (Sarstedt Inc) and then further processed following the manufacturer's instructions for serum collection. Samples were stored at −80C until analysis was performed.

ADA assays were performed on 96-well assay plates (Nunc Plates, Black 96-Well Immuno Plates, Thermo Scientific, 437111) coated with antigen, as follows. A mixture of adalimumab and adalimumab-anti-mouse CD22 scFv fusion protein was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS pH 7.2 for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, 6411-05) was added and incubated for 1 hour at room temperature. After washing the assay plates, 100 μL of QuantaBlu Substrate Solution (Thermo Scientific, 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 21 and 28. Titers were determined by the dilution of serum that gives a 2× OD above background.

### Results and Conclusions

FIG. 16 depicts evaluations of anti-drug antibody responses in mice for adalimumab and adalimumab-anti-mouse CD22 scFv fusion protein. Mice received a single 4 mg/kg i.v. dose of adalimumab or adalimumab-anti-mouse CD22 scFv fusion protein. Serum IgG levels against adalimumab or adalimumab-anti-mCD22 scFv fusion protein were measured at days 20 and 27. Adalimumab-anti-mouse CD22 scFv fusion protein, which is capable of recruiting CD22 to the B cell receptor on adalimumab-specific B cells, is strongly suppressed for anti-drug titer on both day 20 (FIG. 16, panel A) and day 27 (FIG. 16, panel B).

These results for suppression of immunogenicity in mice correlate well with the in vitro B cell activation results in FIG. 8 and FIG. 9 and Examples 7 and 10. As shown previously with antibody conjugates with small molecule Siglec-2 ligands, fusion of an immunogenic protein with a proteinaceous binder of Siglec-2 is also sufficient to strongly suppress immunogenicity.

### Example 16: Synthesis of Compounds

### (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Compound 16-1)

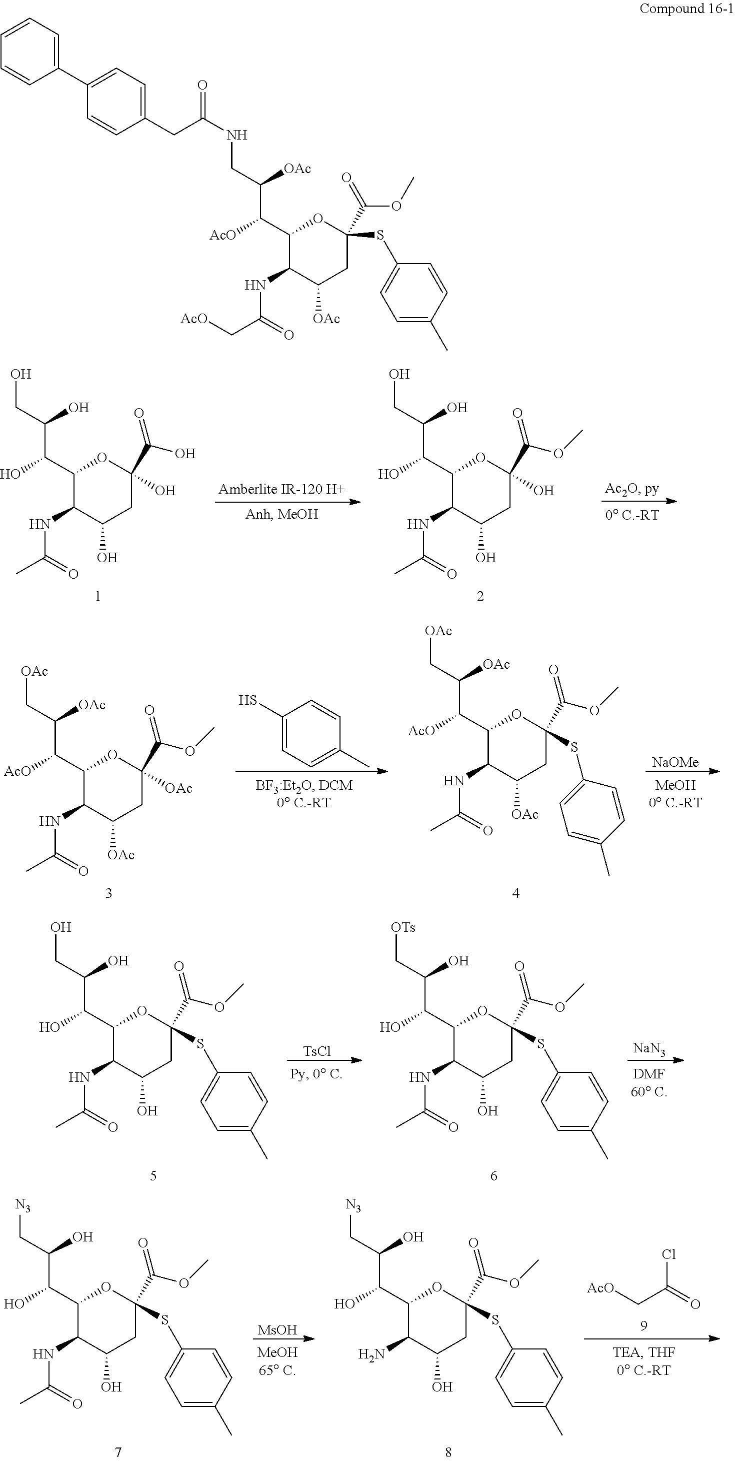

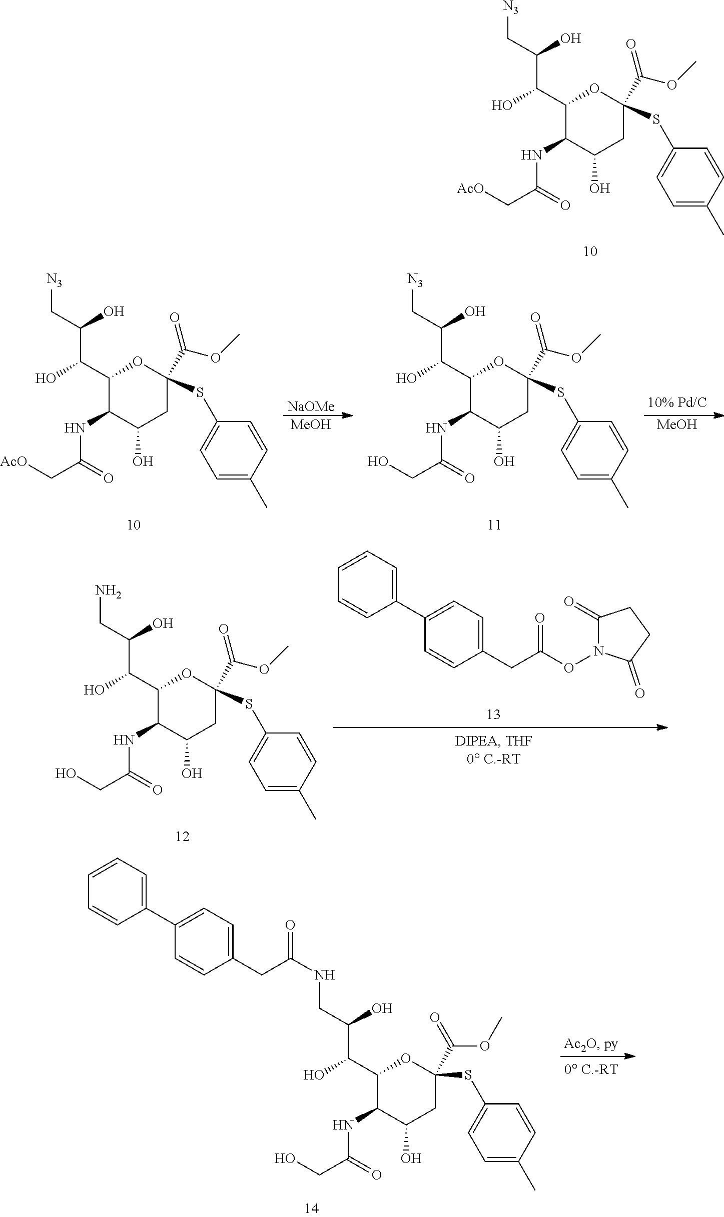

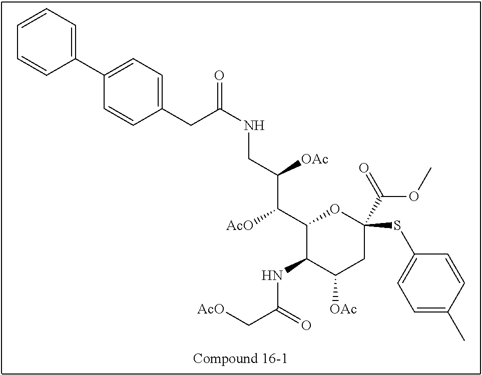 [SMILES: COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(-c3ccccc3)cc2)OC(C)=O)O1]

### Synthesis of methyl (2R,4S,5R,6R)-5-acetamido-2,4-dihydroxy-6-((1R,2R)-1,2,3-trihydroxypropyl)tetrahydro-2H-pyran-2-carboxylate (2)

To a stirred suspension of (2R,4S,5R,6R)-5-acetamido-2,4-dihydroxy-6-((1R,2R)-1,2,3-trihydroxypropyl)tetrahydro-2H-pyran-2-carboxylic acid (1, 100.0 g, 323.3 mmol) in anhydrous methanol (2500 mL) was added Amberlite IR-120 (H+) resin (80.0 g) at room temperature under argon atmosphere. The reaction mixture was stirred under inert atmosphere until the suspension became a clear solution. The resin was removed by filtration and the filtrate was concentrated under reduced pressure to obtain a residue. The residue was triturated with diethyl ether and filtered to afford methyl (2R,4S,5R,6R)-5-acetamido-2,4-dihydroxy-6-((1R,2R)-1,2,3-trihydroxypropyl)tetrahydro-2H-pyran-2-carboxylate (2) as a light pink solid. Yield: 104.0 g, 99.49%; LCMS (ESI) m/z 324.2 [M+H]⁺.

### Synthesis of (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3)

In a 2000 mL round bottom flask, methyl (2R,4S,5R,6R)-5-acetamido-2,4-dihydroxy-6-((1R,2R)-1,2,3-trihydroxypropyl)tetrahydro-2H-pyran-2-carboxylate (2, 102.0 g, 315 mmol) was dissolved with stirring in pyridine (600 mL) under argon atmosphere. To this solution was added acetic anhydride (298 mL, 3.15 mmol) dropwise at 0° C. over 30 min under stirring. The mixture was stirred overnight from 0° C. to room temperature. After completion, the reaction mixture was directly concentrated under reduced pressure on a rotary evaporator. The obtained thick syrup was then poured into a separatory funnel with ethyl acetate (500 mL) and washed with aqueous 1N HCl solution (200 mL) followed by saturated sodium bicarbonate (200 mL) solution and DM water (2×200 mL). The organic layer was separated, dried over anhydrous sodium sulfate, and concentrated under reduced pressure to obtain a thick syrup. The syrup was triturated with diethyl ether and filtered to afford (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3) as a white solid. Yield: 130.0 g, 71.83%; LCMS (ESI) m/z 534.2 [M+H]⁺.

### Synthesis of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4)

Under argon atmosphere, (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3, 130.0 g, 243.68 mmol) was dissolved in anhydrous dichloromethane (1300.0 mL) with stirring. To this solution was added activated powdered 4A molecular sieves (40.0 g). The reaction mixture was stirred at room temperature for 30 min and cooled to 0° C. followed by the dropwise addition of boron trifluoride diethyl etherate (111.0 mL, 365.5 mmol) over 30 min. The mixture was stirred at room temperature. After completion, the reaction mixture was quenched with triethylamine up to neutral pH, filtered over celite, and washed with dichloromethane (100 mL). To the filtrate was added aqueous sodium bicarbonate (300 mL) with stirring. After 10 min, the organic layer was separated, washed with water (2×300 mL), dried over anhydrous sodium sulfate, and concentrated on a rotary evaporator to obtain a crude residue. The obtained crude residue was purified via column chromatography (60-90% ethyl acetate in hexanes) to afford (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4) as a white solid. Yield: 125.0 g, 85.83%; LCMS (ESI) m/z 598.32 [M+H]⁺.

### Synthesis of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5)

To a stirred solution of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4, 100.0 g, 167 mmol) in methanol (800 mL) was slowly added sodium methoxide (25% in MeOH) solution (3.58 mL, 16.7 mmol) at 0° C. The reaction mixture was stirred for 2 h at room temperature. After completion, the reaction mixture was cooled to 0° C. and quenched with DOWEX hydrogen form to maintain pH 6. The mixture was filtered through celite and concentrated under reduced pressure to obtain solids that were then triturated with diethyl ether and filtered to afford methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5) as an off white solid. Yield: 71.0 g, 98.80%; LCMS (ESI) m/z 430.10 [M+H]⁺.

### Synthesis of methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6)

To a stirred solution of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5, 40.0 g, 93.1 mmol) in pyridine (300 mL) was dropwise added a solution of 4-methylbenzene-1-sulfonyl chloride (30.2 g, 158 mmol) in pyridine (100 mL) at 0° C. The resulting reaction solution was stirred overnight. After completion, the reaction mixture was directly concentrated under reduced pressure to obtain a thick syrup. The thick syrup was purified via flash column chromatography (80-95% ethyl acetate in hexanes) to afford methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6) as a white solid. Yield: 21.2 g, 39.0%. LC-MS (ESI) m/z 584.05 [M+H]⁺.

### Synthesis of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7)

In an inert atmosphere, methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6, 21.0 g, 35.98 mmol) was dissolved under stirring in anhydrous N,N-dimethylformamide (210.0 mL).

To this solution was added sodium azide (7.80 g, 120 mmol) at room temperature. The resulting reaction mixture was stirred at 60° C. for 16 h. After completion, the reaction mixture was directly concentrated on a rotary evaporator to obtain a crude solid. The crude solid was purified via column chromatography (80-95% ethyl acetate in hexanes) to afford methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7) as an off white solid. Yield: 11.7 g, 71.55%; LCMS (ESI) m/z 453.19 [M−H]⁻.

### Synthesis of methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8)

To a stirred solution of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7, 21.0 g, 46.20 mmol) in methanol (210.0 mL) was added methane sulfonic acid (18.0 mL, 277.2 mmol) dropwise at 0° C. The resulting reaction mixture was stirred at 65° C. for 30 h. After completion, the reaction mixture was cooled to 0° C. and quenched with triethylamine (˜15.0 mL, pH 7). The mixture was concentrated under reduced pressure to afford crude methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8) as a light brown gel. Yield: 19.0 g, 99.68%; LC-MS (ESI) m/z 413.57 [M+H]⁺.

### Synthesis of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (10)

In an inert atmosphere, crude methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8, 19.0 g, 46.06 mmol) was dissolved under stirring in dry tetrahydrofuran (200.0 mL) and cooled to 0° C. To this solution was slowly added triethylamine (17.70 mL, 138.2 mmol) followed by 2-chloro-2-oxoethyl acetate (9, 4.95 mL, 46.06 mmol) at 0° C. The reaction was stirred at 0° C. for 3 h. The mixture was concentrated under reduced pressure to obtain a crude residue which was then purified via column chromatography (60-75% ethyl acetate in hexanes) to afford methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (10) as a white solid. Yield: 15.2 g, 64.38%; LCMS (ESI) m/z 513.42 [M+H]⁺.

### Synthesis of methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (11)

To a stirred solution of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (10, 15.0 g, 29.27 mmol) in methanol (150.0 mL) at 0° C. was slowly added sodium methoxide solution (25% in methanol, 0.061 ml, 2.93 mmol). The reaction mixture was stirred for 1 h at room temperature. The reaction mixture was cooled to 0° C. and quenched with DOWEX hydrogen form to maintain pH 6. The mixture was filtered through celite and concentrated under reduced pressure to obtain solids that were triturated with diethyl ether and filtered on a centered funnel to afford methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (11) as an off white solid. Yield: 13.0 g, 94.41%; LCMS (ESI) m/z, 471.15 [M+H]⁺.

### Synthesis of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (12)

To a stirred solution of methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (11, 13.0 g, 27.63 mmol) in methanol (130 mL) was added 10% Pd/C (13.0 g, 100% w/w) at room temperature. The reaction was then hydrogenated using balloon pressure of H₂ gas for 12 h. After completion, the reaction was filtered through celite and the filtrate was concentrated. The obtained residue was then dried under high vacuum to afford crude methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (12) as a thick syrup. Yield: 12.2 g, 99.41%; LCMS (ESI) m/z 445.16 [M+H]⁺.

### Synthesis of methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (14)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (12, 12.2 g, 27.45 mmol) and 2,5-dioxocyclopentyl 2-([1,1′-biphenyl]-4-yl)acetate (13, 10.19 g, 32.94 mmol) in tetrahydrofuran (40.0 mL) was added ethylbis(propan-2-yl)amine (22.4 mL, 137.23 mmol) at 0° C. The resulting reaction mixture was stirred at room temperature for 12 h.

After completion, the mixture was concentrated under reduced pressure to obtain a crude residue. The crude residue was purified via column chromatography (80-90% ethyl acetate in hexanes) to afford methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (14) as a white solid. Yield: 8.0 g, 45.63%; LCMS (ESI) m/z 639.23 [M+H]⁺.

### Synthesis of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Compound 16-1)

To a stirred solution of methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (14, 8.0 g, 12.52 mmol) in pyridine (80.0 mL) was dropwise added acetic anhydride (11.61 mL, 125.2 mmol) at 0° C. over 30 min. The reaction mixture was stirred overnight from 0° C. to room temperature. After completion, volatiles were removed under vacuum to obtain a crude thick syrup. The crude thick syrup was then poured into a separatory funnel with ethyl acetate (240.0 mL) and washed with 1N HCl solution followed by saturated sodium sulfate solution. The organic layer was dried over anhydrous sodium sulfate and concentrated under reduced pressure to obtain a crude thick syrup. The crude thick syrup was purified via column chromatography (60-70% ethyl acetate in hexanes) to afford (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Compound 16-1) as a white solid. Yield: 5.40 g, 53.43%; LCMS (ESI) m/z 807.2 [M+H]⁺. 1H NMR (400 MHz, methanol-d₄) δ 7.27 (d, J=8.4 Hz, 2H), 7.14 (d, J=8.4 Hz, 2H), 4.03 (t, J=8.4 Hz, 1H), 3.77 (d, J=7.6 Hz, 2H), 3.51-3.47 (m, 2H), 3.20 (t, J=6.4 Hz, 2H), 2.91 (dd, J=9.6, 14.4 Hz, 1H), 2.82 (dd, J=6, 14 Hz, 1H), 2.24-2.20 (m, 3H), 2.07 (d, J=9.6 Hz, 1H), 1.75 (d, J=12.8 Hz, 2H), 1.68-1.62 (m, 2H), 1.60-1.57 (m, 2H), 1.56-1.47 (m, 1H).

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-2) and (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-3)

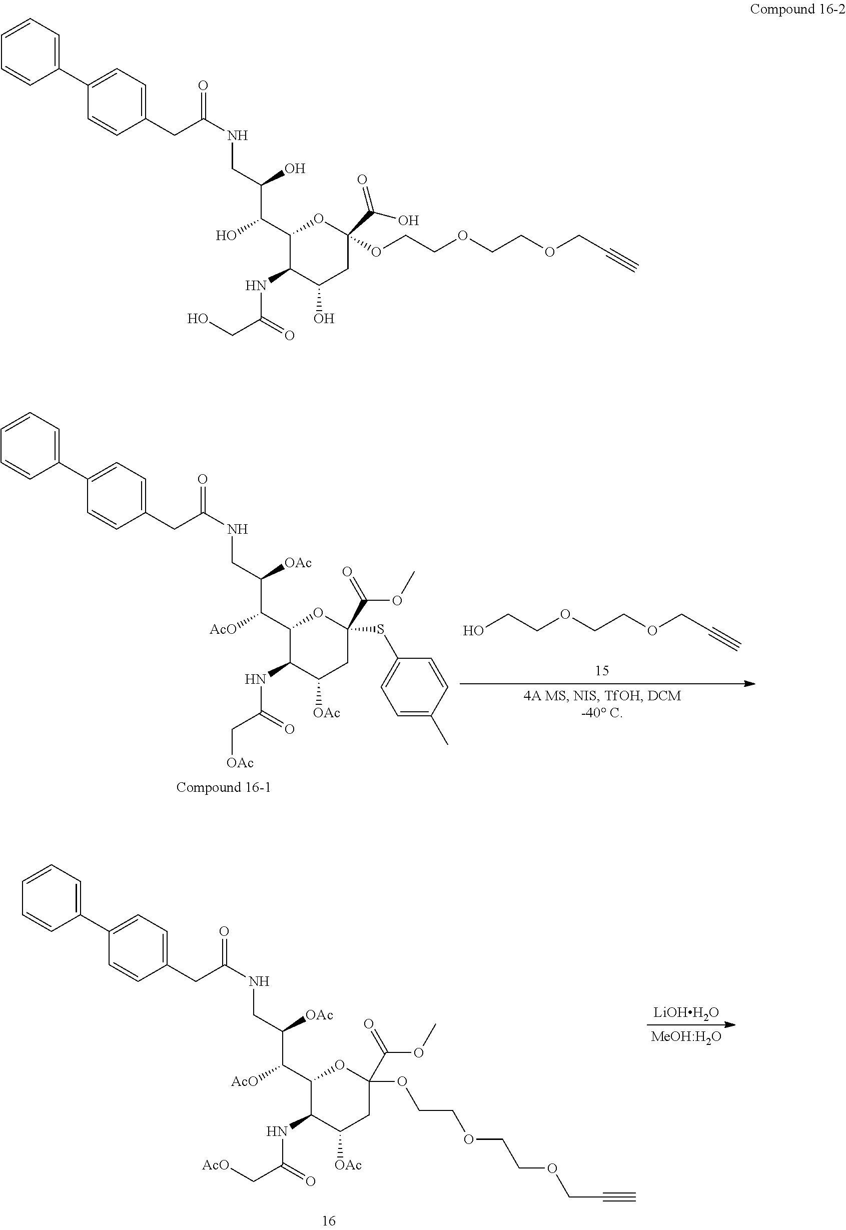

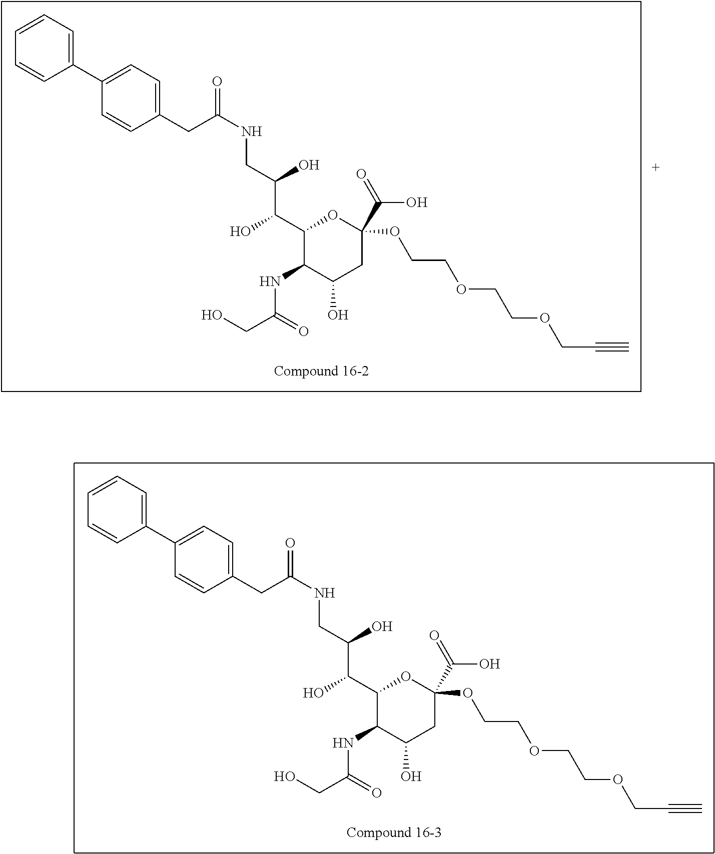 [SMILES: C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; C#CCOCCOCCO[C@@]1(C(=O)O)C[C@H](O)[C@@H](NC(=O)CO)[C@H]([C@H](O)[C@H](O)CNC(=O)Cc2ccc(-c3ccccc3)cc2)O1]

### Synthesis of (1R,2R)-3-(2-([1,1′-biphenyl]-4-yl)acetamido)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (16)

To a stirred solution of (1R,2R)-3-(2-([1,1′-biphenyl]-4-yl)acetamido)-1-((2R,3R,4S,6S)-4-acetoxy-3-(2-acetoxyacetamido)-6-(methoxycarbonyl)-6-(p-tolylthio)tetrahydro-2H-pyran-2-yl)propane-1,2-diyl diacetate (Compound 16-1, 1.0 g, 1.240 mmol) in anhydrous dichloromethane (20.0 mL) was added 2-(2-(prop-2-yn-1-yloxy)ethoxy)ethan-1-ol (15, 0.893 g, 6.20 mmol) and activated 4A powdered molecular sieves (1.0 g, 100% w/w). The resulting reaction solution was stirred at 15 h at room temperature under nitrogen. To the solution was added 1-iodopyrrolidine-2,5-dione (0.697 g, 3.10 mmol) and trifluoromethanesulfonic acid (0.109 mL, 1.240 mmol) at −40° C. The resulting reaction solution was stirred at −40° C. for 1 h. After completion, the reaction mixture was quenched with triethyl amine (0.5 mL) and warmed to room temperature. The reaction mixture was filtered through a sintered funnel and washed with dichloromethane. The filtrate was washed with saturated sodium bicarbonate (aq), dried over sodium sulfate, filtered, and concentrated under reduced pressure to obtain a crude residue. The residue was purified via column chromatography (60-80% ethyl acetate in hexanes) to afford (1R,2R)-3-(2-([1,1′-biphenyl]-4-yl)acetamido)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (16) as a white solid as an anomeric mixture. Yield: 0.80 g, 78.07%; LCMS (ESI) m/z 827.30 [M+H]⁺.

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-2) and (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-3)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3, 0.450 g, 0.544 mmol) in methanol (5.0 mL) was added a solution of Lithium hydroxide monohydrate (0.137 g, 3.27 mmol) in water (0.50 mL). The resulting reaction mixture was stirred at room temperature for 6 h. After completion, the reaction mixture was treated with Dowex 50, H+) up to pH˜6 and the suspension was filtered and washed with methanol. The filtrate was concentrated under reduced pressure to obtain a crude residue. The residue was purified via preparatory HPLC to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-2) as a white solid. Yield: 0.316 g, 90%; LCMS (ESI) m/z 645.45 [M+H]⁺. 1H NMR (400 MHz, Methanol-d₄) δ 7.87 (d, J=7.6 Hz, 1H), 7.61-7.56 (m, 4H), 7.43-7.37 (m, 4H), 7.31 (t, J=7.2 Hz, 1H), 4.17 (d, J=2.4 Hz, 2H), 3.90 (s, 2H), 3.89-3.81 (m, 4H), 3.74-3.59 (m, 11H), 3.39-3.25 (m, 2H), 2.82 (t, J=0.8 Hz, 1H), 2.71 (dd, J=8.8 & 4.0 Hz, 1H), 1.75 (t, J=12.4 Hz, 1H); (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-3) as a white solid. Yield: 0.028 g, 8.0%; LCMS (ESI) m/z 645.42 [M+H]⁺ 0.1H NMR (400 MHz, Methanol-d₄) δ 7.82 (d, J=8.8 Hz, 1H), 7.61-7.57 (m, 4H), 7.44-7.36 (m, 4H), 7.31 (t, J=7.2 Hz, 1H), 4.17 (d, J=2.4 Hz, 2H), 4.15-4.119 (m, 1H), 4.03-3.96 (m, 3H), 3.94-3.86 (m, 1H), 3.86-3.78 (m, 2H), 3.36-3.62 (m, 2H) 3.59 (s, 2H), 3.51-3.46 (m, 1H), 3.42-3.35 (m, 2H), 2.84 (t, J=2.4 Hz, 1H), 2.38 (dd, J=12.8 & 3.8 Hz, 1H), 1.66 (t, J=11.6 Hz, 1H).

### (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Compound 16-4)

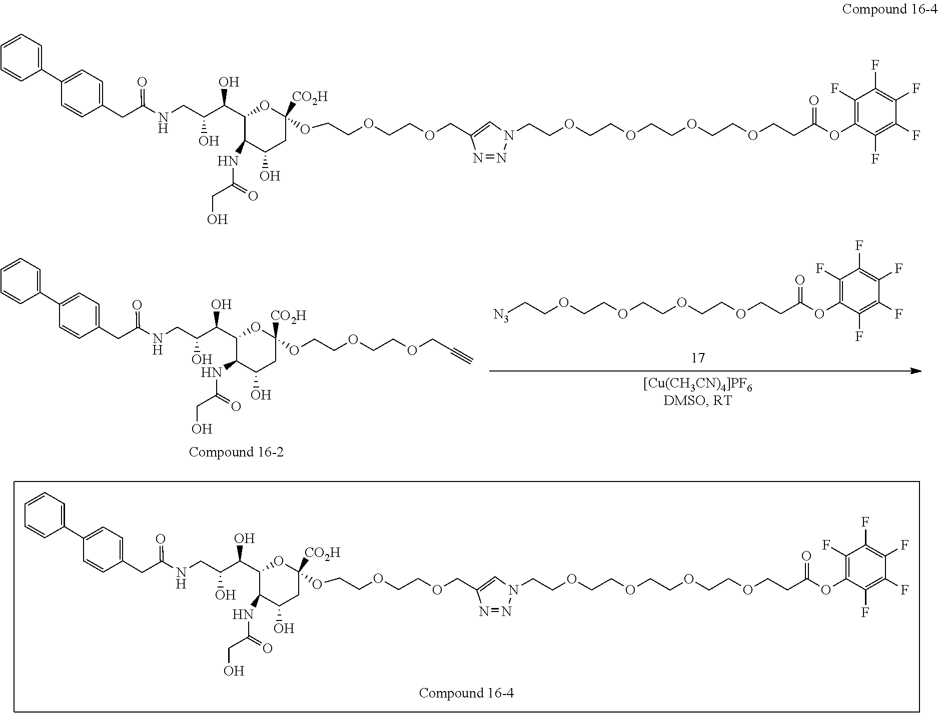 [SMILES: O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; [N-]=[N+]=NCCOCCOCCOCCOCCC(=O)Oc1c(F)c(F)c(F)c(F)c1F ; O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO]

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Compound 16-4)

To a stirred solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 16-2, 0.035 g, 0.054 mmol) and perfluorophenyl 1-azido-3,6,9,12-tetraoxapentadecan-15-oate (17, 0.025 g, 0.054 mmol) in dimethyl sulfoxide (0.5 mL) was added tetrakis(acetonitrile) copper(I) hexafluorophosphate (0.050 g, 0.135 mmol). The resulting reaction mixture was stirred at room temperature for 30 min. After completion, acetic acid (0.3 mL) was added. The resulting solution was diluted with acetonitrile and purified via preparatory HPLC (19-35% acetonitrile in water with 0.1% TFA). Fractions containing the desired product were combined and lyophilized to dryness to afford (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Compound 16-4) as the TFA salt as a white solid. Yield: 0.022 g, 36.77%; LCMS (ESI) m/z 1102.72 [M+H]⁺; 1H NMR (400 MHz, DMSO-d₆ with D₂O exchange) δ 7.98 (d, J=3.6 Hz, 2H), 7.59 (d, J=7.2 Hz, 2H), 7.56-7.52 (m, 2H), 7.42 (t, J=8.0 Hz, 2H), 7.33-7.31 (m, 3H), 4.46 (s, 4H), 4.11 (m, 2H), 3.55-3.40 (m, 22H), 3.19 (d, J=9.2 Hz, 2H), 2.99-2.92 (m, 5H), 2.45-2.43 (m, 6H), 2.50 (m, 1H), 1.20 (s, 1H).

### Embodiments of the Invention

Embodiments of the present invention include but are not limited to the following clauses.

1. An engineered autoantigen comprising an autoantigen which has been engineered to comprise a modified Sialic acid-binding immunoglobulin-type lectin (Siglec) ligand profile relative to a corresponding unengineered autoantigen.

2. The engineered autoantigen according to clause 1, wherein the Siglec ligand profile comprises an elevated amount of one or more Siglec ligands covalently bound to the engineered autoantigen relative to the corresponding unengineered autoantigen.

3. The engineered autoantigen according to clause 1 or 2, wherein one or more of the Siglec ligands is a ligand for a B cell-associated Siglec.

4. The engineered autoantigen according to clause 3, wherein the B-cell associated Siglec is selected from the group consisting of Siglec-2 (CD22), Siglec-5 (CD170), Siglec-6, Siglec-9 (CD329) and Siglec-10 (Siglec G).

5. The engineered autoantigen according to clause 4, wherein the sialic acid is a naturally occurring sialic acid.

6. The engineered autoantigen according to clause 5, wherein the Siglec ligand is a naturally occurring Siglec ligand.

7. The engineered autoantigen according to clause 5, wherein the Siglec ligand is a non-naturally occurring Siglec ligand.

8. The engineered autoantigen according to clause 7, wherein the non-naturally occurring Siglec ligand further comprises a non-naturally occurring linker.

9. The engineered autoantigen according to clause 8, wherein the non-naturally occurring Siglec ligand consists essentially of the naturally occurring sialic acid bound to the non-naturally occurring linker.

10. The engineered autoantigen according to clause 8 or 9, wherein the linker does not comprise a saccharide.

11. The engineered autoantigen according to clause 4, wherein the Siglec ligand is a non-naturally occurring Siglec ligand that comprises a non-naturally occurring sialic acid.

12. The engineered autoantigen according to clause 11, wherein the non-naturally occurring sialic acid is covalently bound to the autoantigen.

13. The engineered autoantigen according to clause 11, wherein the non-naturally occurring Siglec ligand further comprises a non-naturally occurring linker.

14. The engineered autoantigen according to clause 13, wherein the non-naturally occurring Siglec ligand consists essentially of the non-naturally occurring sialic acid covalently bound to the non-naturally occurring linker.

15. The engineered autoantigen according to clause 13 or 14, wherein the linker does not comprise a saccharide.

16. The engineered autoantigen according to clause 4, wherein the Siglec ligand comprises two sialic acids and a linker, wherein the linker is a branched linker and the two sialic acids are attached to the linker.

17. The engineered autoantigen according to clause 4, wherein the Siglec ligand comprises three sialic acids and a linker, wherein the linker is a branched linker and the three sialic acids are attached to the linker.

18. The engineered autoantigen according to clause 16 or 17, wherein the linker does not comprise a natural saccharide.

19. The engineered autoantigen according to any one of clauses 1-18, wherein the autoantigen comprises a polypeptide or a nucleic acid.

20. The engineered autoantigen according to any one of clauses 1-4, wherein the autoantigen comprises a polypeptide and the Siglec ligand comprises a Siglec binding polypeptide.

21. The engineered autoantigen according to clause 20, wherein the Siglec binding polypeptide comprises the amino acid sequence RNDYTE (SEQ ID NO:3).

22. The engineered autoantigen according to any one of clauses 1-21, wherein the autoantigen comprises Siglec ligands for both Siglec-2 and Siglec-10.

23. The engineered autoantigen according to any one of clauses 1-22, wherein the engineered autoantigen comprises one or more Siglec ligands and the corresponding unengineered immunogenic autoantigen comprises less Siglec ligands than the engineered autoantigen.

24. The engineered autoantigen according to clause 23, wherein the engineered autoantigen comprises one or more Siglec ligands and the corresponding unengineered immunogenic autoantigen comprises no Siglec ligands.

25. The engineered autoantigen according to any one of clauses 1-24, wherein the autoantigen comprises 2-fold more Siglec ligand than a corresponding unengineered immunogenic autoantigen present in an individual having an autoimmune disorder and to which the individual produces an immune response.

26. The engineered autoantigen according to any one of clauses 1-24, wherein the autoantigen comprises 3-fold more Siglec ligand than a corresponding unengineered autoantigen present in an individual having an autoimmune disorder and to which the individual produces an immune response.

27. The engineered autoantigen according to any one of clauses 1-23, wherein the autoantigen comprises 5-fold more Siglec ligand than a corresponding unengineered autoantigen present in an individual having an autoimmune disorder and to which the individual produces an immune response.

28. The engineered autoantigen according to any one of clauses 1-24, wherein the autoantigen comprises 10-fold more Siglec ligand than a corresponding unengineered autoantigen that induces an antibody response in an individual against the autoantigen.

29. The engineered autoantigen according to any one of clauses 1-28, wherein the engineered autoantigen elicits an autoantigen-specific antibody titer that is 50% or less of the autoantigen-specific antibody titer that would be elicited by a corresponding unengineered autoantigen in an individual comprising the autoantigen.

30. A method of making a autoantigen, the method comprising covalently attaching a sialic acid to an autoantigen to create an engineered autoantigen.

31. The method according to clause 30, wherein the covalently attaching comprises sialylation by engineered biosynthesis.

32. The method according to clause 30, wherein the covalently attaching comprises sialylation by chemical conjugation.

33. The method according to clause 32, wherein the chemical conjugation of the sialic acid is to a glycan of the autoantigen.

34. The method according to clause 33, wherein the chemical conjugation of the sialic acid to the glycan of the autoantigen results in a covalent bond between the sialic acid and the glycan.

35. The method according to clause 33 or 34, wherein the chemical conjugation of the sialic acid to the glycan of the biotherapeutic incorporates a linker between the sialic acid and the glycan.

36. The method according to clause 32, wherein the chemical conjugation of the sialic acid is to an amino acid of the autoantigen.

37. The method according to clause 36, wherein the chemical conjugation of the sialic acid to the amino acid of the autoantigen results in a covalent bond between the sialic acid and the amino acid.

38. The method according to clause 37, wherein the chemical conjugation of the sialic acid to the amino acid of the autoantigen incorporates a linker between the sialic acid and the amino acid.

39. The method according to any one of clauses 30-38, wherein the sialic acid is a naturally occurring sialic acid.

40. The method according to any one of clauses 30-38, wherein the sialic acid is a non-naturally occurring sialic acid.

41. A method for generating an engineered autoantigen comprising a polypeptide autoantigen and a siglec binding polypeptide according to any one of clauses 20-29, wherein the method comprises expressing the engineered autoantigen in vivo or in vitro from a nucleic acid encoding the engineered autoantigen, wherein the polypeptide autoantigen and the siglec binding polypeptide retain ability to bind to a B-cell receptor and a Siglec, respectively, expressed on a B-cell.

42. The method according to clause 41, wherein the Siglec binding polypeptide comprises or consists of the sequence RNDYTE (SEQ ID NO:3).

43. The method according to clause 41 or 42, wherein the Siglec is selected from the group consisting of Siglec-2 (CD22), Siglec-5 (CD170), Siglec-6, Siglec-9 (CD329) and Siglec-10 (Siglec G).

44. The method according to any one of clauses 41-43, wherein the engineered autoantigen comprises a single siglec binding polypeptide.

45. The method according to any one of clauses 41-43, wherein the engineered autoantigen comprises two siglec binding polypeptides.

46. The method according to clause 45, wherein the two siglec binding polypeptides are the same.

47. The method according to clause 45, wherein the two siglec binding polypeptides are different.

48. The method according to any one of clauses 41-47, wherein the siglec binding polypeptide is fused to the N-terminus or the C-terminus of the autoantigen.

49. A nucleic acid encoding the engineered autoantigen according to any one of clauses 20-29.

50. A host cell comprising the nucleic acid according to clause 49.

51. The host cell of clause 50, wherein the host cell comprises a vector comprising the nucleic acid according to clause 49.

52. The host cell of clause 51, wherein the vector is a viral vector.

53. The host cell of clause 52, wherein the host cell is capable of producing infectious recombinant adenoassociated virus (rAAV) virions comprising the nucleic acid.

54. A pharmaceutical composition, comprising:
an engineered autoantigen according to any one of clauses 1-29 or a composition manufactured according to any one of clauses 30-48; or
a nucleic acid encoding the engineered autoantigen according to any one of clauses 20-29 or a composition manufactured according to any one of clauses 41-48;
rAAV virions comprising the nucleic acid encoding the engineered autoantigen according to any one of clauses 20-29;
and
a pharmaceutical excipient.

55. A method of treating an individual having an autoimmune disease comprising an immune response to an antigen, the method comprising administering to the individual the engineered autoantigen according to any one of clauses 1-29, the nucleic acid of clause 49, or the pharmaceutical composition according to clause 54 in an amount effective to treat the autoimmune disorder or disease, wherein the administering results in reduction of antibodies against the antigen in the individual.

56. The method according to clause 55, wherein the autoimmune disease or disorder is selected from the group consisting of Pemphigus vulgaris, celiac disease, primary biliary cholangitis, membranous nephropathy, rheumatoid arthritis, Systemic lupus erythematosus rheumatoid arthritis, psoriatic arthritis, ankylosing spondylitis, Crohn's disease, ulcerative colitis, psoriasis, hidradenitis suppurativa, uveitis, and juvenile idiopathic arthritis.

57. The method according to clause 55 or 56, wherein
the autoimmune disorder is Pemphigus vulgaris and the autoantigen is Desmoglein (Dsg) 1 and/or Dsg 3,
the autoimmune disorder is celiac disease and the autoantigen is Gliadin and/or type 2 transglutaminase,
the autoimmune disorder is primary biliary cholangitis and the autoantigen is Pyruvate dehydrogenase complex component E2 (PDC-E2),
the autoimmune disorder is membranous neuropathy and the autoantigen is PLA2R (Phospholipase A2 Receptor),
the autoimmune disorder is myasthenia gravis and the autoantigen is muscle-specific kinase (MuSK), low-density lipoprotein receptor-related protein 4 (LRP4), and/or Acetylcholine Receptor (AChR),
the autoimmune disorder is Graves' disease and the autoantigen is thyroid-stimulating hormone receptor (TSHR),
the autoimmune disorder is rheumatoid arthritis and the autoantigen is citrullinated autoantigen, carbamylated autoantigen, and/or acetylated autoantigen, or
the autoimmune disorder is Systemic lupus erythematosus and the autoantigen is dsDNA, nucleosomes, ribonucleoprotein, and/or Sm antigen.

58. The method according to any one of clauses 55-57, wherein the method further comprises:
drawing serum from the individual 8 weeks after administering the engineered autoantigen and assessing the serum for autoantigen-specific antibodies.

59. The method according to any one of clauses 55-58, wherein the autoantigen is administered to an individual for 1 month or more.

60. The method according to any one of clauses 55-58, wherein the autoantigen is administered to an individual for 3 months or more.

61. The method according to any one of clauses 55-58, wherein the autoantigen is administered to an individual for 6 months or more.

62. The method according to any one of clauses 55-58, wherein the autoantigen is administered to an individual for 1 year or more.

63. The method according to any one of clauses 55-62, wherein the administration to the individual is weekly.

64. The method according to any one of clauses 55-62, wherein the administration to the individual is biweekly.

65. The method according to any one of clauses 55-62, wherein the administration to the individual is monthly.

66. The method according to any one of clauses 55-62, wherein the administration to the individual is quarterly or semi-annually.

67. The method according to any one of clauses 55-58, wherein the administration to the individual is annually or bi-annually.

68. The method according to any one of clauses 55-67, wherein the autoantigen-specific antibody titer is measured 8 weeks after the last administration of the autoantigen.

69. An engineered autoantigen, comprising an autoantigen covalently bound to a nonnaturally occurring Siglec ligand, wherein the Siglec ligand comprises
a non-naturally occurring sialic acid selected from the group consisting of

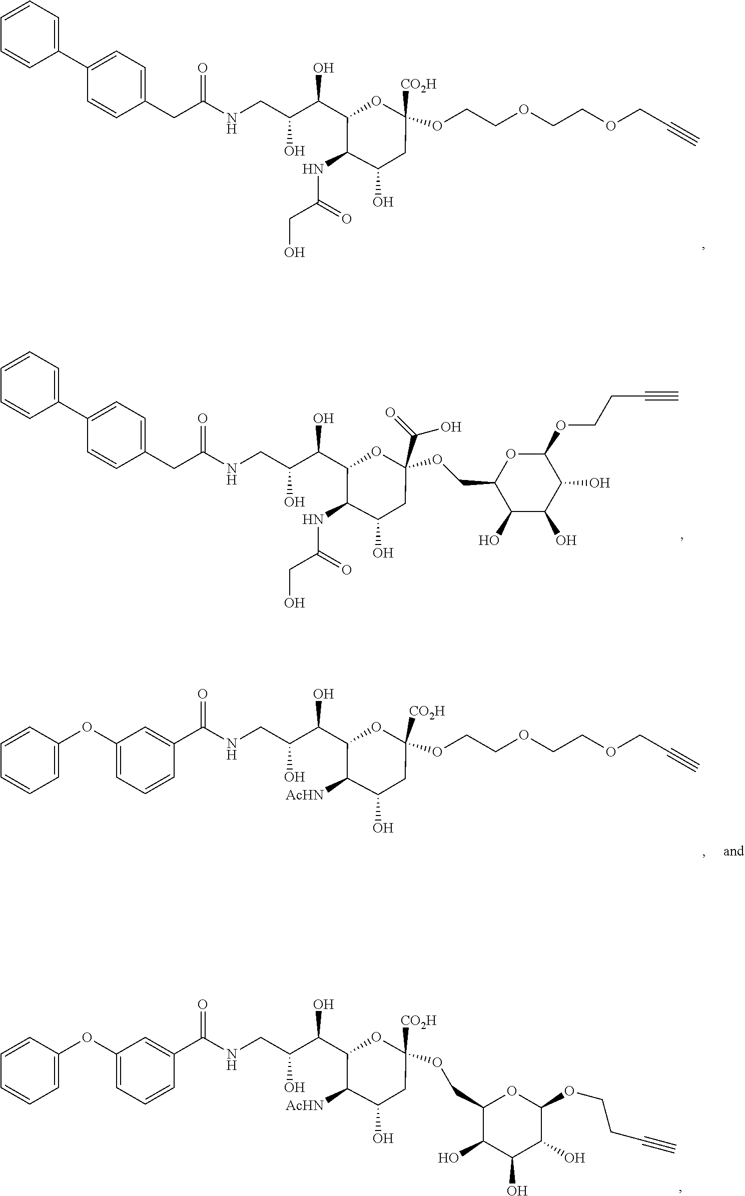 [SMILES: C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)cc2)O1 ; C#CCCO[C@@H]1O[C@H](CO[C@]2(C(=O)O)C[C@H](O)[C@@H](NC(=O)CO)[C@H]([C@H](O)[C@H](O)CNC(=O)Cc3ccc(-c4ccccc4)cc3)O2)[C@H](O)[C@H](O)[C@H]1O ; C#CCOCCOCCO[C@]1(C(=O)O)C[C@H](O)[C@@H](NC(C)=O)[C@H]([C@H](O)[C@H](O)CNC(=O)c2cccc(Oc3ccccc3)c2)O1 ; C#CCCO[C@@H]1O[C@H](CO[C@]2(C(=O)O)C[C@H](O)[C@@H](NC(C)=O)[C@H]([C@H](O)[C@H](O)CNC(=O)c3cccc(Oc4ccccc4)c3)O2)[C@H](O)[C@H](O)[C@H]1O]

and a linker selected from the group consisting of

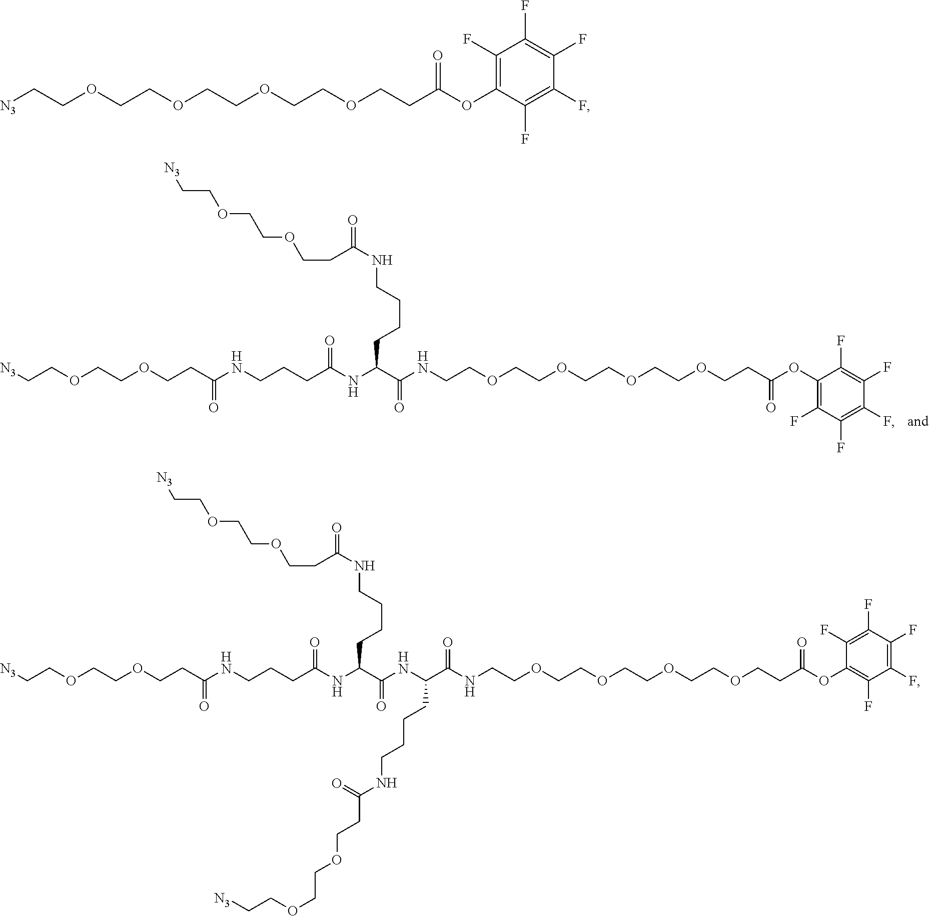 [SMILES: [N-]=[N+]=NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F]

wherein the linker attaches the sialic acid to the autoantigen.

70. The engineered autoantigen according to clause 69, wherein the nonnaturally occurring Siglec ligand is selected from the group consisting of:

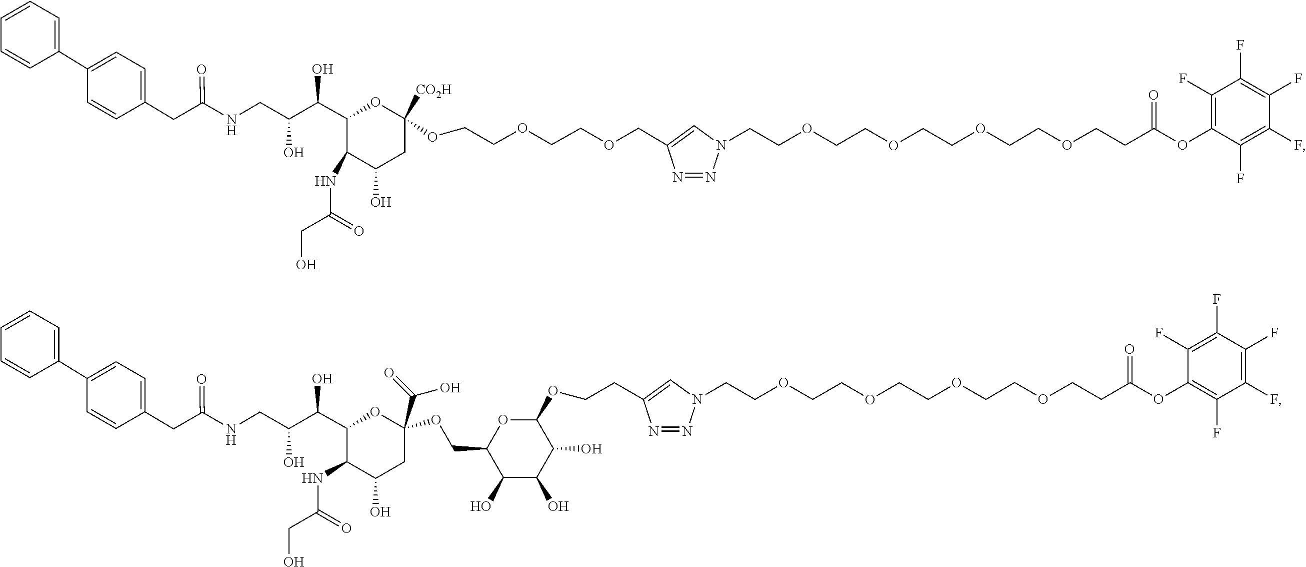 [SMILES: O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; O=C(Cc1ccc(-c2ccccc2)cc1)NC[C@@H](O)[C@@H](O)[C@@H]1O[C@@](OC[C@H]2O[C@@H](OCCc3cn(CCOCCOCCOCCOCCC(=O)Oc4c(F)c(F)c(F)c(F)c4F)nn3)[C@H](O)[C@@H](O)[C@H]2O)(C(=O)O)C[C@H](O)[C@H]1NC(=O)CO]

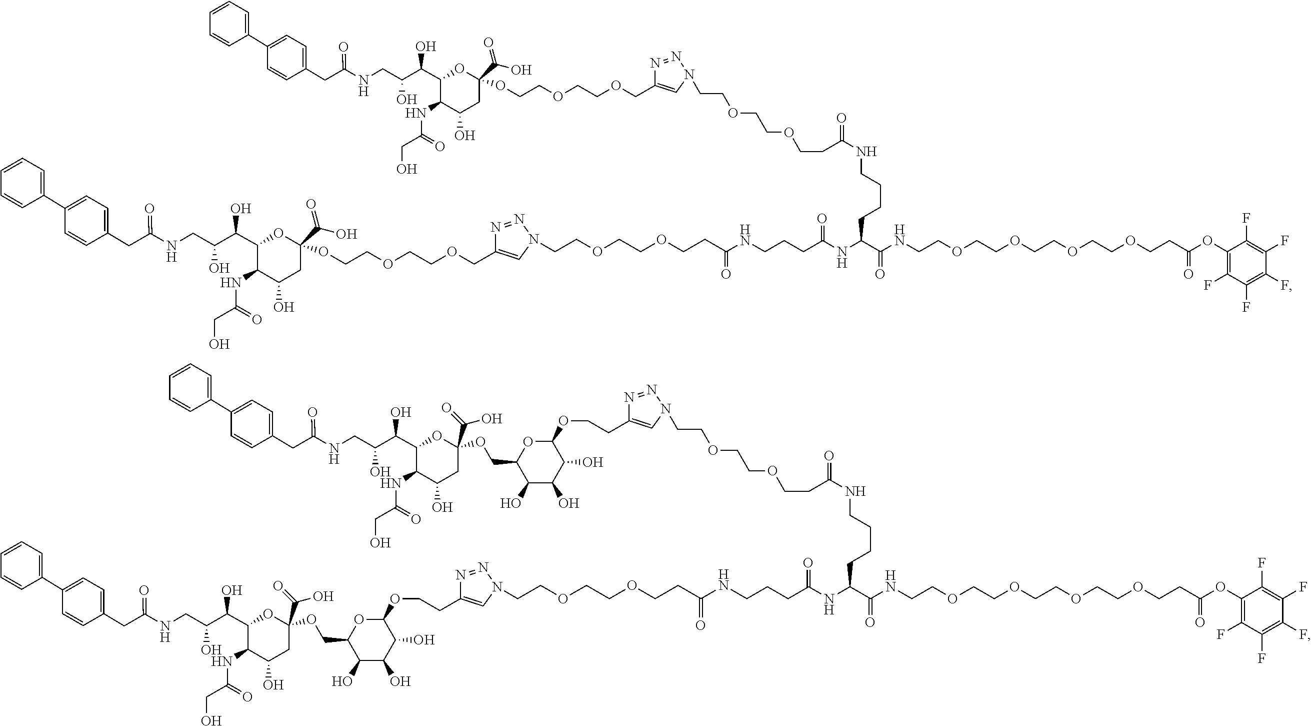

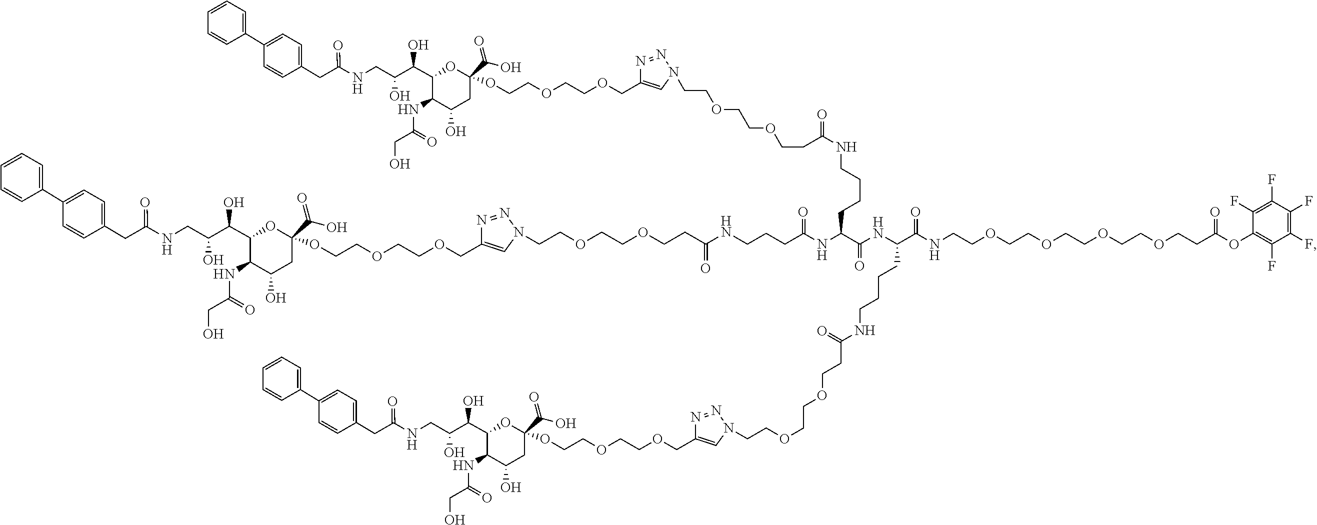 [SMILES: O=C(CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC[C@H](NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

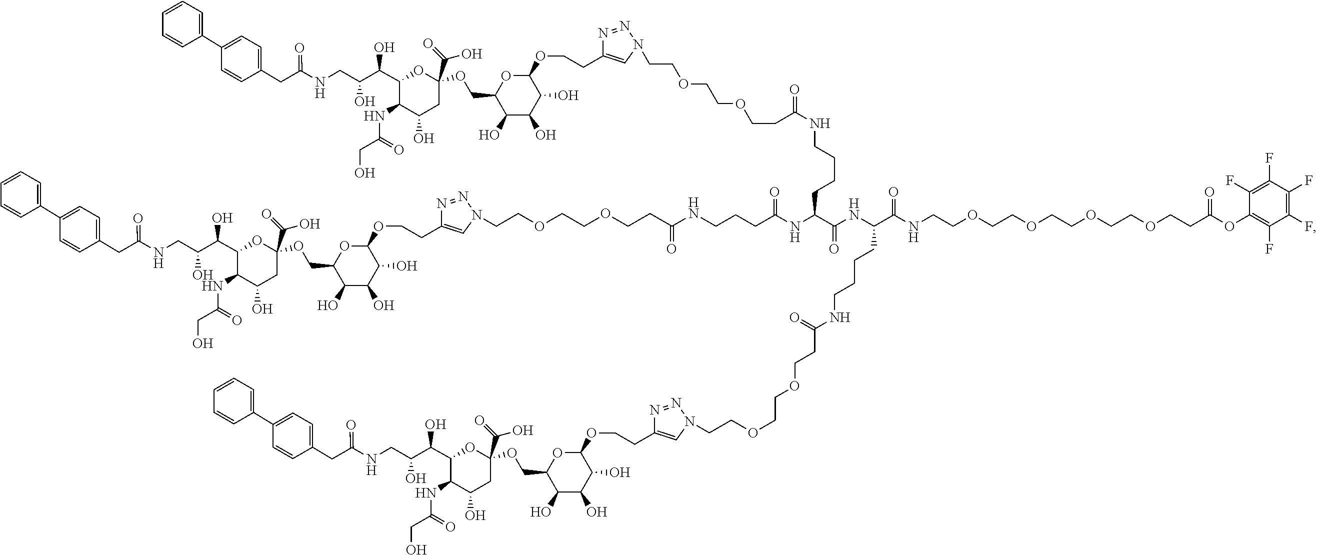

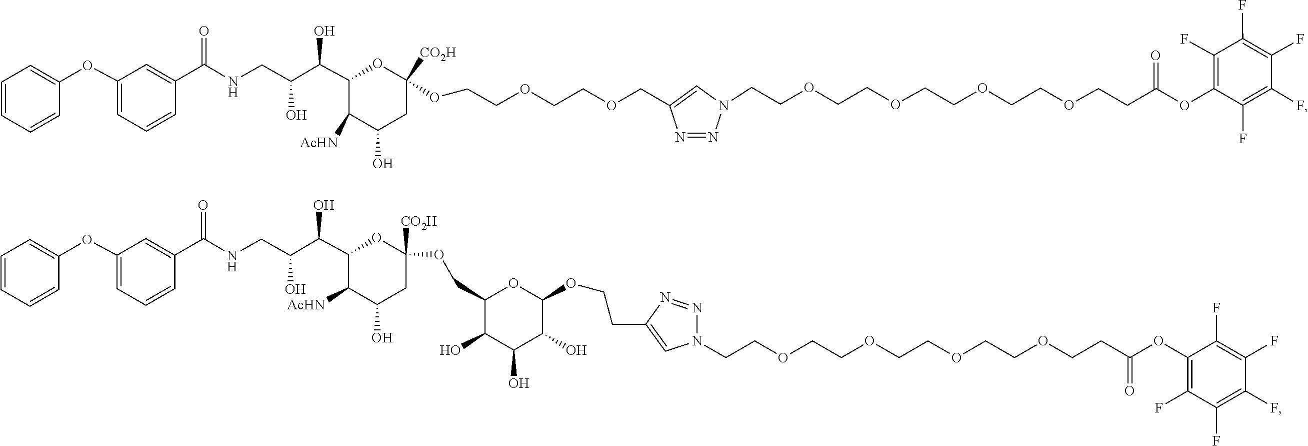 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCOCCOCCC(=O)Oc3c(F)c(F)c(F)c(F)c3F)nn2)(C(=O)O)C[C@@H]1O ; CC(=O)N[C@H]1[C@H]([C@H](O)[C@H](O)CNC(=O)c2cccc(Oc3ccccc3)c2)O[C@@](OC[C@H]2O[C@@H](OCCc3cn(CCOCCOCCOCCOCCC(=O)Oc4c(F)c(F)c(F)c(F)c4F)nn3)[C@H](O)[C@@H](O)[C@H]2O)(C(=O)O)C[C@@H]1O]

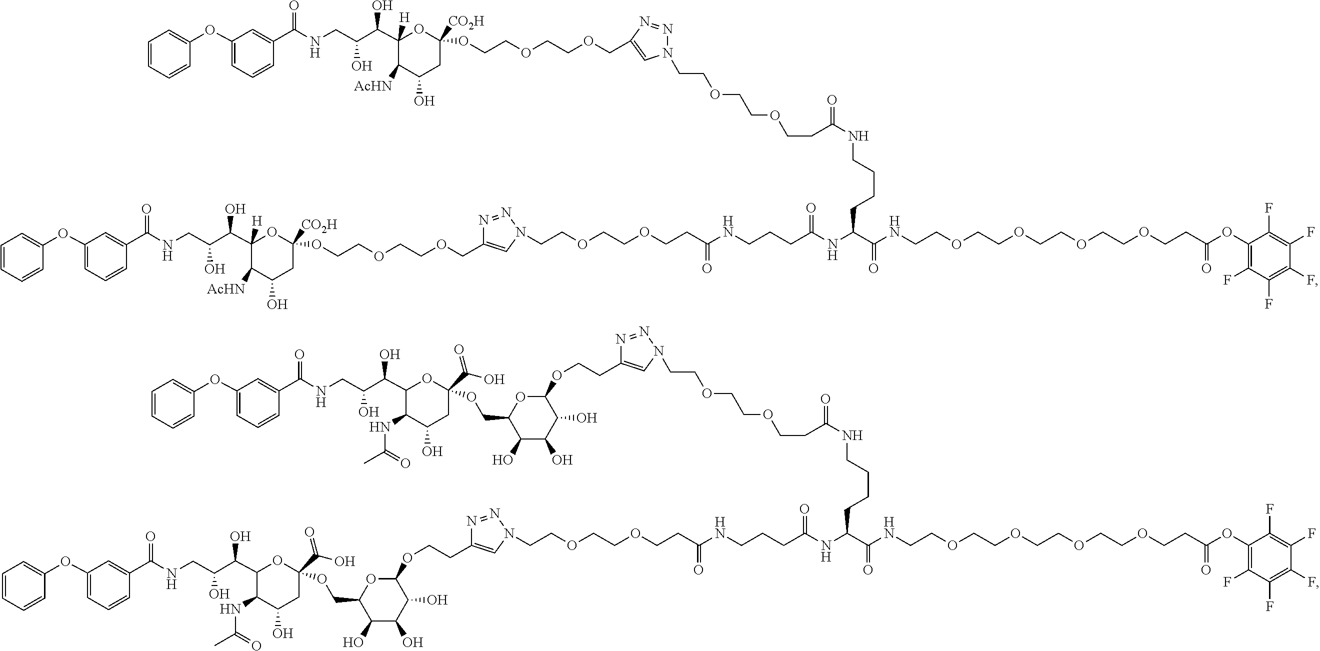

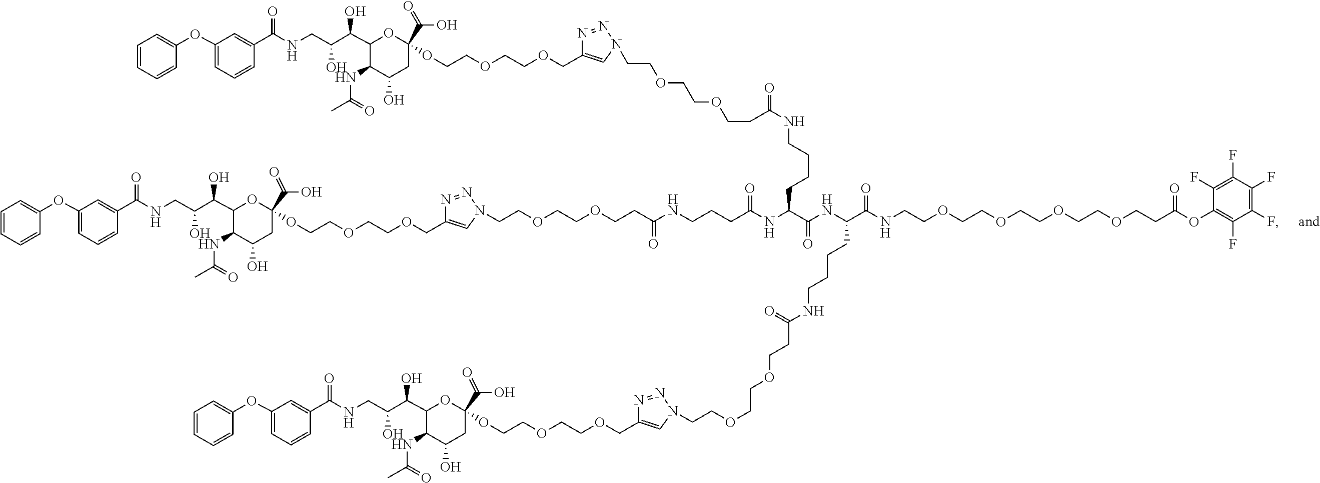 [SMILES: CC(=O)N[C@H]1C([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)C([C@H](O)[C@H](O)CNC(=O)c5cccc(Oc6ccccc6)c5)O4)nn3)C(=O)N[C@@H](CCCCNC(=O)CCOCCOCCn3cc(COCCOCCO[C@]4(C(=O)O)C[C@H](O)[C@@H](NC(C)=O)C([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O]

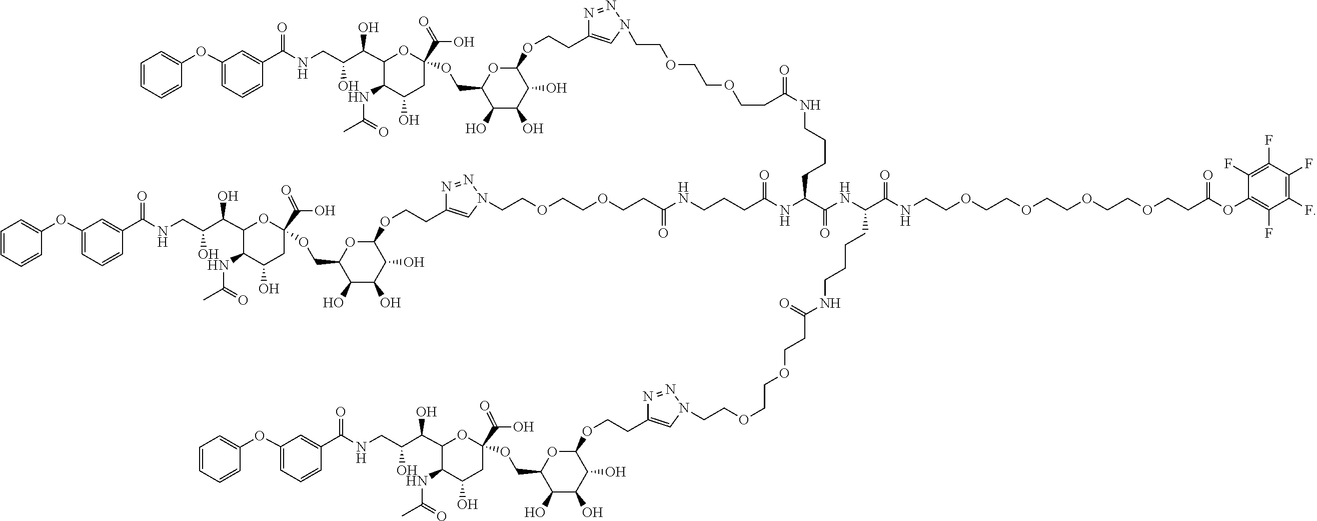

71. The engineered autoantigen according to clause 69 or 70, wherein the nonnaturally occurring Siglec ligand does not comprise a saccharide between the linker and the sialic acid.

72. An engineered autoantigen of formula (I):

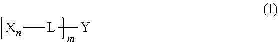

wherein X is a sialic acid group, L is an optional linker, Y is an autoantigen, n is an integer of 1 or more, and m is an integer of 1 or more.

73. A nucleic acid encoding a hypoimmunogenic biotherapeutic, wherein the hypoimmunogenic biotherapeutic is a fusion protein comprising a therapeutically active polypeptide and a siglec binding protein or peptide, wherein the siglec binding protein or peptide binds to a B-cell associated siglec and wherein binding of the siglec binding protein to the siglec suppresses B-cell activation.

74. The nucleic acid according to clause 73, wherein the nucleic acid is present in a vector.

75. The nucleic acid according to clause 74, wherein the vector is a viral vector.

76. The nucleic acid according to any one of clauses 73-75, wherein the siglec binding protein binds to a Siglec is selected from the group consisting of Siglec-2 (CD22), Siglec-5 (CD170), Siglec-6, Siglec-9 (CD329) and Siglec-10 (Siglec G).

78. The nucleic acid according to clause 76, wherein the siglec binding protein binds to CD22 and comprises the amino acid sequence RNDYTE (SEQ ID NO:3).

79. The nucleic acid according to any one of clauses 73-78, wherein the biotherapeutic is an antibody is selected from the group consisting of a monoclonal antibody, a bispecific antibody, an scFv, a Fab, a camelid, or a nanobody, optionally, wherein the antibody is selected from the group consisting of adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, and tocilizumab..

80. The nucleic acid according to any one of clauses 73-78, wherein the biotherapeutic is selected from the group consisting of erythropoietin, thrombopoietin, human growth hormone, tissue factor, IFNβ-1b, IFNβ3-1a, IL-2 or the IL-2 mimetic aldesleukin, exenatide, albiglutide, alefacept, palifermin, and belatacept.

81. The nucleic acid according to any one of clauses 73-78, wherein the biotherapeutic is an enzyme selected from the group consisting of asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), Tripeptidyl peptidase (TPP1), Pseudomonas elastase (PaE), Pseudomonas alkaline protease (PaAP), and Streptococcal pyrogenic exotoxin B (SpeB).

82. A recombinant AAV virion comprising the nucleic acid of any one of clauses 73-81.

83. The rAAV virion according to clause 82, comprising a capsid VP1 protein selected from the group consisting of an AAV1, AAV2, AAV3b, AAV4, AAV5, AAV6, AAV7, AAV8, AAV9, AAVrh10, AAV11, AAV12, and AAV13 VP1 protein, or a variant thereof.

84. A pharmaceutical composition comprising the nucleic acid according to any one of clauses 73-81 or the rAAV virion according to clause 82 or 83.

85. A method for suppressing an immune response to an antigen in a subject having the immune response, the method comprising:
administering to the subject an engineered antigen comprising the antigen that has been engineered to comprise a modified Sialic acid-binding immunoglobulin-type lectin (Siglec) ligand profile relative to a corresponding unengineered antigen.

86. The method of clause 85, wherein suppressing an immune response comprises preventing an increase in immune response as compared to the immune response in absence of the administering.

87. The method of clause 85, wherein suppressing an immune response comprises decreasing the immune response to the antigen as compared to the immune response in absence of the administering.

88. The method of any one of clauses 85-87, wherein the immune response comprises a B-cell mediated immune response.

89. The method of clause 88, wherein the B-cell mediated immune response comprises IgG antibodies that bind to the antigen.

90. The method of any one of clauses 85-89, wherein the antigen in an autoantigen, wherein the autoantigen is produced by the subject from an endogenous gene and wherein the subject comprises antibodies against the autoantigen.

91. The method of any one of clauses 85-89, wherein the antigen in biotherapeutic, wherein the biotherapeutic is not encoded by the subject from an endogenous gene, wherein the subject has been previously administered the biotherapeutic as a replacement therapy and comprises antibodies against the biotherapeutic.

92. The method of any one of clauses 85-91, wherein antigen portion of the engineered antigen comprises a fragment of the antigen, wherein the fragment includes an epitope bound by an antibody produced by the subject.

93. The method of clause 92, wherein the fragment comprises an amino acid sequence of an immunodominant region of the antigen.

94. The method of clause 92 or 93, wherein the fragment is a fusion of two separate regions of the antigen.

95. The method according to any one of clauses 85-94, wherein the antigen is administered to an individual for 1 month or more, 3 months or more, 6 months or more, or 1 year or more.

96. The method according to any one of clauses 85-95, wherein the administration to the individual is weekly, biweekly, monthly, quarterly or semi-annually, or annually or bi-annually.

97. The method according to any one of clauses 85-96, wherein the Siglec ligand profile comprises an elevated amount of one or more Siglec ligands covalently bound to the engineered antigen relative to the corresponding unengineered antigen.

98. The method according to any one of clauses 85-97, wherein one or more of the Siglec ligands is a ligand for a B cell-associated Siglec.

99. The method according to any one of clauses 85-98, wherein the B-cell associated Siglec is selected from the group consisting of Siglec-2 (CD22), Siglec-5 (CD170), Siglec-6, Siglec-9 (CD329) and Siglec-10 (Siglec G).

100. The method according to clause 99, wherein the sialic acid is a naturally occurring sialic acid.

101. The method according to clause 100, wherein the Siglec ligand is a naturally occurring Siglec ligand.

102. The method according to clause 100, wherein the Siglec ligand is a non-naturally occurring Siglec ligand.

103. The method according to clause 102, wherein the non-naturally occurring Siglec ligand further comprises a non-naturally occurring linker.

104. The method according to clause 103, wherein the non-naturally occurring Siglec ligand consists essentially of the naturally occurring sialic acid bound to the non-naturally occurring linker.

105. The method according to clause 103 or 104, wherein the linker does not comprise a saccharide.

106. The method according to clause 99, wherein the Siglec ligand is a non-naturally occurring Siglec ligand that comprises a non-naturally occurring sialic acid.

107. The method according to clause 106, wherein the non-naturally occurring sialic acid is covalently bound to the antigen.

108. The method according to clause 106, wherein the non-naturally occurring Siglec ligand further comprises a non-naturally occurring linker.

109. The method according to clause 108, wherein the non-naturally occurring Siglec ligand consists essentially of the non-naturally occurring sialic acid covalently bound to the non-naturally occurring linker.

110. The method according to clause 107 or 108, wherein the linker does not comprise a saccharide.

111. The method according to clause 99, wherein the Siglec ligand comprises two sialic acids and a linker, wherein the linker is a branched linker and the two sialic acids are attached to the linker.

112. The method according to clause 99, wherein the Siglec ligand comprises three sialic acids and a linker, wherein the linker is a branched linker and the three sialic acids are attached to the linker.

113. The method according to clause 111 or 112, wherein the linker does not comprise a natural saccharide.

114. The method according to any one of clauses 85-113, wherein the antigen comprises a polypeptide or a nucleic acid.

115. The method according to any one of clauses 85-99, wherein the antigen comprises a polypeptide and the Siglec ligand comprises a Siglec binding polypeptide.

116. The method according to clause 115, wherein the Siglec binding polypeptide comprises the amino acid sequence RNDYTE (SEQ ID NO:3).

117. The method according to any one of clauses 85-116, wherein the antigen comprises Siglec ligands for both Siglec-2 and Siglec-10.

118. The method according to any one of clauses 85-117, wherein the engineered antigen comprises one or more Siglec ligands and the corresponding unengineered antigen comprises less Siglec ligands than the engineered antigen.

119. The method according to clause 118, wherein the engineered antigen comprises one or more Siglec ligands and the corresponding unengineered antigen comprises no Siglec ligands.

120. The method according to any one of clauses 85-119, wherein the antigen comprises 2-fold more, 3-fold more, 5-fold more, or 10-fold more Siglec ligand than a corresponding unengineered antigen present in an individual having an autoimmune disorder and to which the individual produces an immune response.

121. The method according to any one of clauses 85-120, wherein the engineered antigen elicits an antigen-specific antibody titer that is 50% or less of the antigen-specific antibody titer that would be elicited by a corresponding unengineered antigen in the individual.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

The scope of the present invention, therefore, is not intended to be limited to the exemplary embodiments shown and described herein. Rather, the scope and spirit of the present invention is embodied by the appended claims.

1.-120. (canceled)

121. An engineered autoantigen comprising an autoantigen which has been engineered to comprise an elevated amount of one or more Sialic acid-binding immunoglobulin-type lectin (Siglec) ligand relative to a corresponding unengineered autoantigen.

122. The engineered autoantigen of claim 121, wherein the engineered autoantigen is of formula (I): 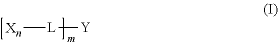
wherein X is a sialic acid group, L is an optional linker, Y is an autoantigen, n is an integer of 1 or more, and m is an integer of 2 or more.

123. The engineered autoantigen of claim 122, wherein m is an integer of 3 or more.

124. The engineered autoantigen of claim 121, wherein the Siglec ligand comprises a linker that does not include a saccharide.

125. The engineered autoantigen of claim 121, wherein the Siglec ligand comprises a sialic acid and a linker and wherein the Siglec ligand does not comprise a saccharide between the linker and the sialic acid.

126. The engineered autoantigen of claim 122, wherein X has the structure of formula (II): 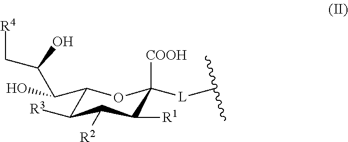
wherein
R¹, R², R³ and R⁴ are each independently selected from hydrogen, alkyl, substituted alkyl, alkenyl, substituted alkenyl, alkynyl, substituted alkynyl, hydroxyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, cycloalkyl, substituted cycloalkyl, heterocyclyl, and substituted heterocyclyl, aryl, substituted aryl, heteroaryl, and substituted heteroaryl.

127. The engineered autoantigen of claim 122, wherein L comprises a polyethylene glycol group.

128. The engineered autoantigen of claim 122, wherein L is a branched linker comprising one or more branching groups.

129. The engineered autoantigen of claim 121, wherein the one or more Siglec ligands comprise:

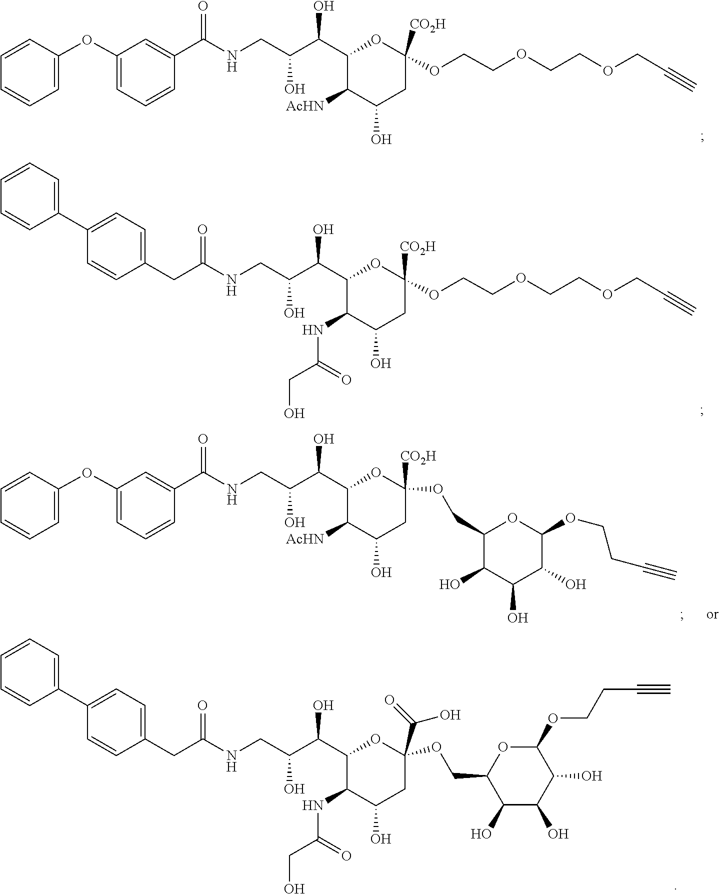

130. The engineered autoantigen of claim 121, wherein the Siglec ligand comprises a linker comprising: 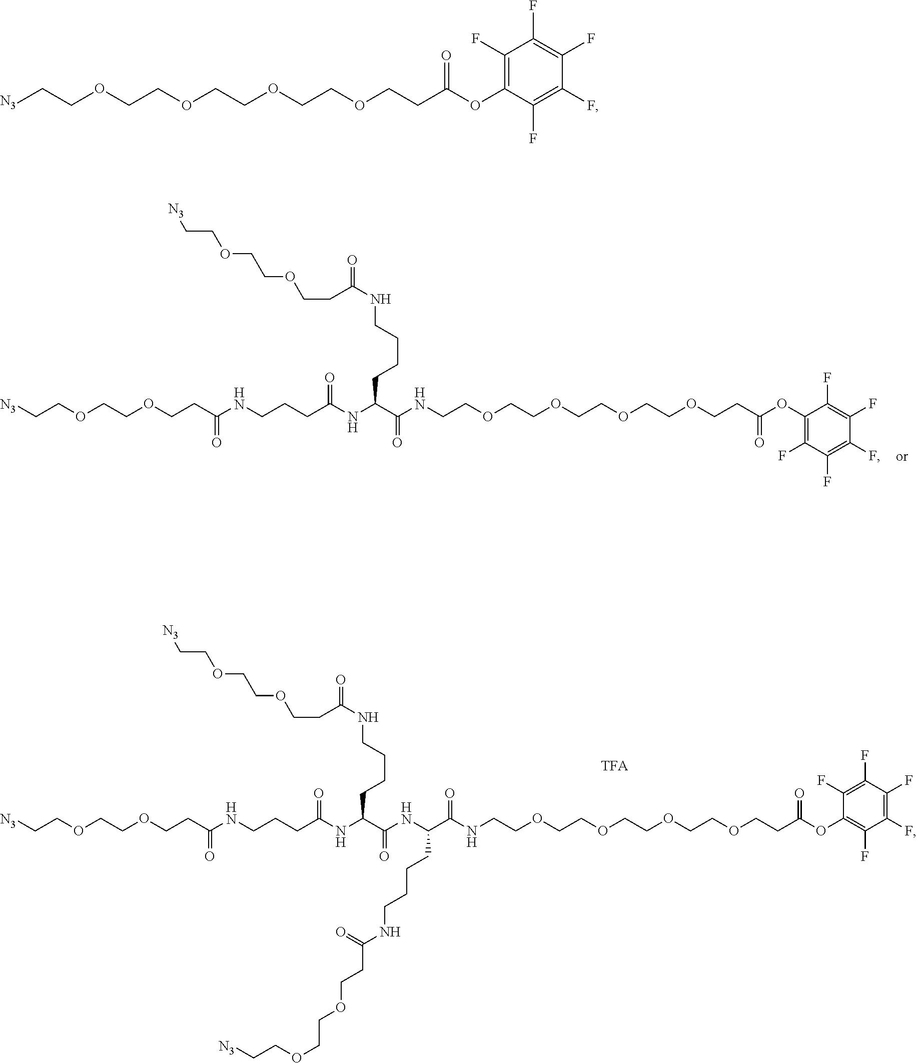 [SMILES: [N-]=[N+]=NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)NCCOCCOCCOCCOCCC(=O)Oc1c(F)c(F)c(F)c(F)c1F ; [N-]=[N+]=NCCOCCOCCC(=O)NCCCC[C@H](NC(=O)CCCNC(=O)CCOCCOCCN=[N+]=[N-])C(=O)N[C@@H](CCCCNC(=O)CCOCCOCCN=[N+]=[N-])C(=O)NCCOCCOCCOCCOCCC(=O)Oc1c(F)c(F)c(F)c(F)c1F]
wherein the linker attaches the sialic acid to the biotherapeutic.

131. The engineered autoantigen of claim 121, wherein the Siglec ligand comprises:

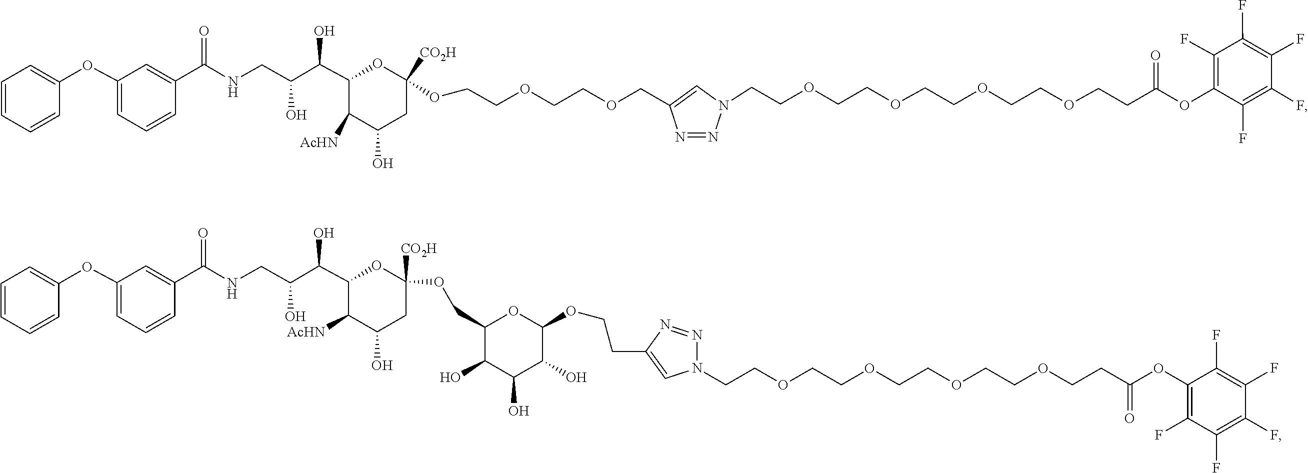 [SMILES: CC(=O)N[C@H]1[C@H]([C@H](O)[C@H](O)CNC(=O)c2cccc(Oc3ccccc3)c2)O[C@@](OCCOCCOCc2cn(CCOCCOCCOCCOCCC(=O)Oc3c(F)c(F)c(F)c(F)c3F)nn2)(C(=O)O)C[C@@H]1O ; CC(=O)N[C@H]1[C@H]([C@H](O)[C@H](O)CNC(=O)c2cccc(Oc3ccccc3)c2)O[C@@](OC[C@H]2O[C@@H](OCCc3cn(CCOCCOCCOCCOCCC(=O)Oc4c(F)c(F)c(F)c(F)c4F)nn3)[C@H](O)[C@@H](O)[C@H]2O)(C(=O)O)C[C@@H]1O]

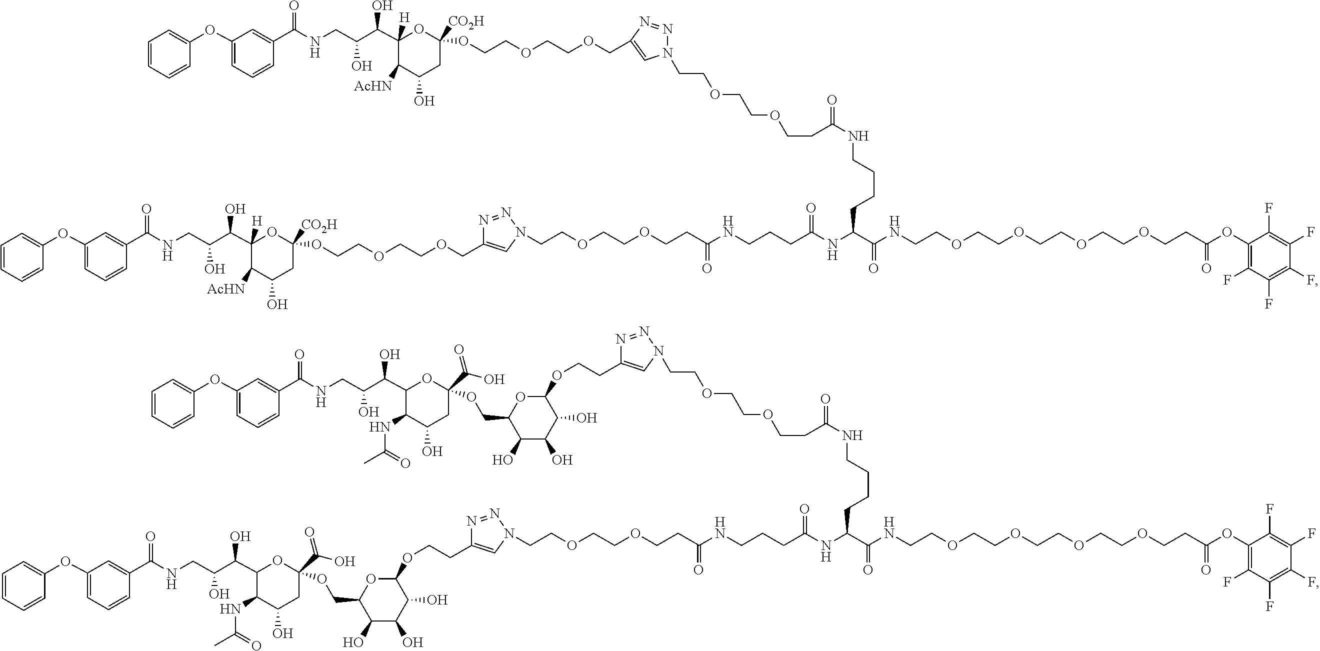

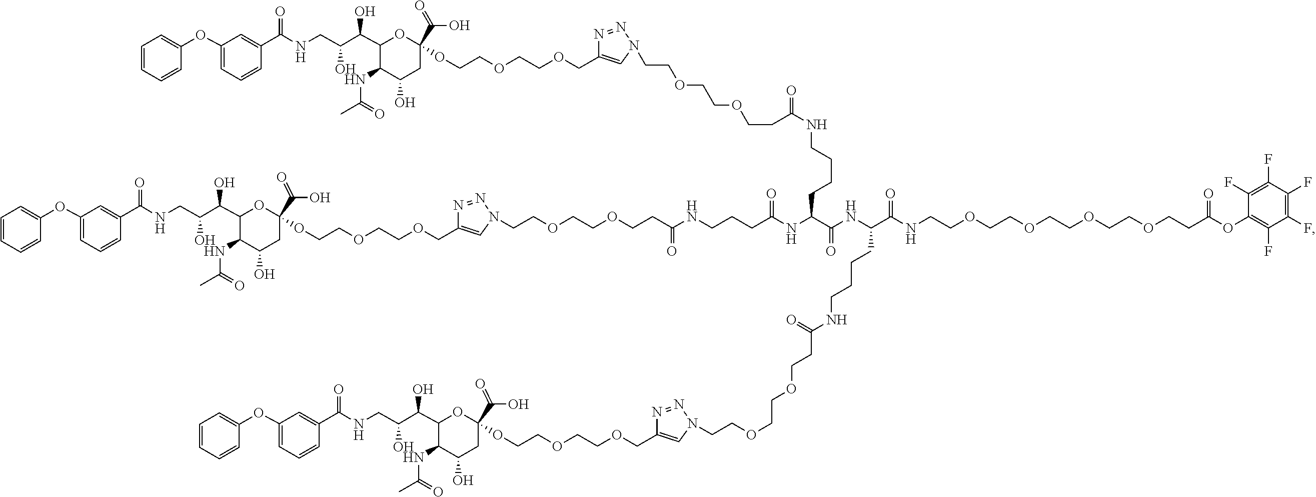 [SMILES: CC(=O)N[C@H]1C([C@H](O)[C@H](O)CNC(=O)c2cccc(Oc3ccccc3)c2)O[C@@](OCCOCCOCc2cn(CCOCCOCCC(=O)NCCCC[C@H](NC(=O)CCCNC(=O)CCOCCOCCn3cc(COCCOCCO[C@]4(C(=O)O)C[C@H](O)[C@@H](NC(C)=O)C([C@H](O)[C@H](O)CNC(=O)c5cccc(Oc6ccccc6)c5)O4)nn3)C(=O)N[C@@H](CCCCNC(=O)CCOCCOCCn3cc(COCCOCCO[C@]4(C(=O)O)C[C@H](O)[C@@H](NC(C)=O)C([C@H](O)[C@H](O)CNC(=O)c5cccc(Oc6ccccc6)c5)O4)nn3)C(=O)NCCOCCOCCOCCOCCC(=O)Oc3c(F)c(F)c(F)c(F)c3F)nn2)(C(=O)O)C[C@@H]1O]

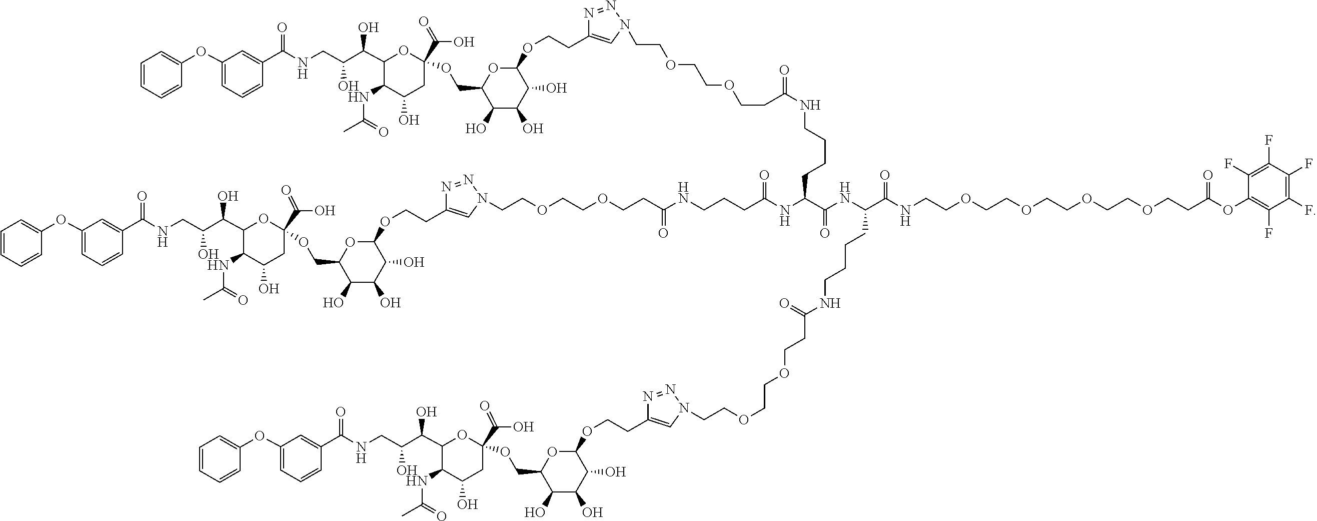

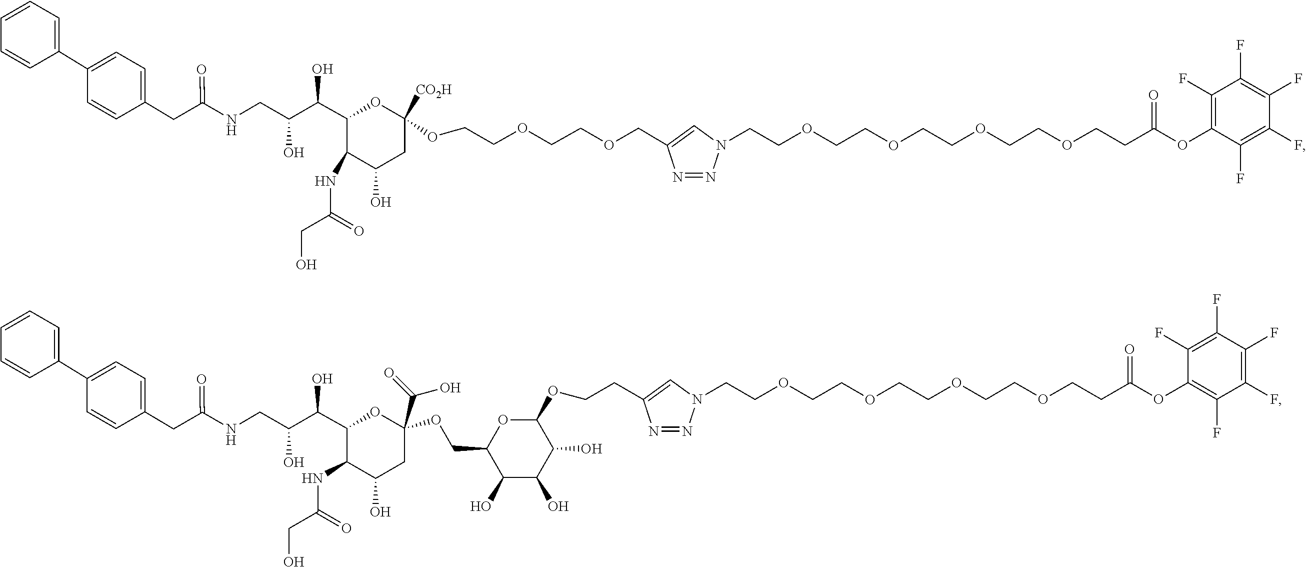 [SMILES: O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; O=C(Cc1ccc(-c2ccccc2)cc1)NC[C@@H](O)[C@@H](O)[C@@H]1O[C@@](OC[C@H]2O[C@@H](OCCc3cn(CCOCCOCCOCCOCCC(=O)Oc4c(F)c(F)c(F)c(F)c4F)nn3)[C@H](O)[C@@H](O)[C@H]2O)(C(=O)O)C[C@H](O)[C@H]1NC(=O)CO]

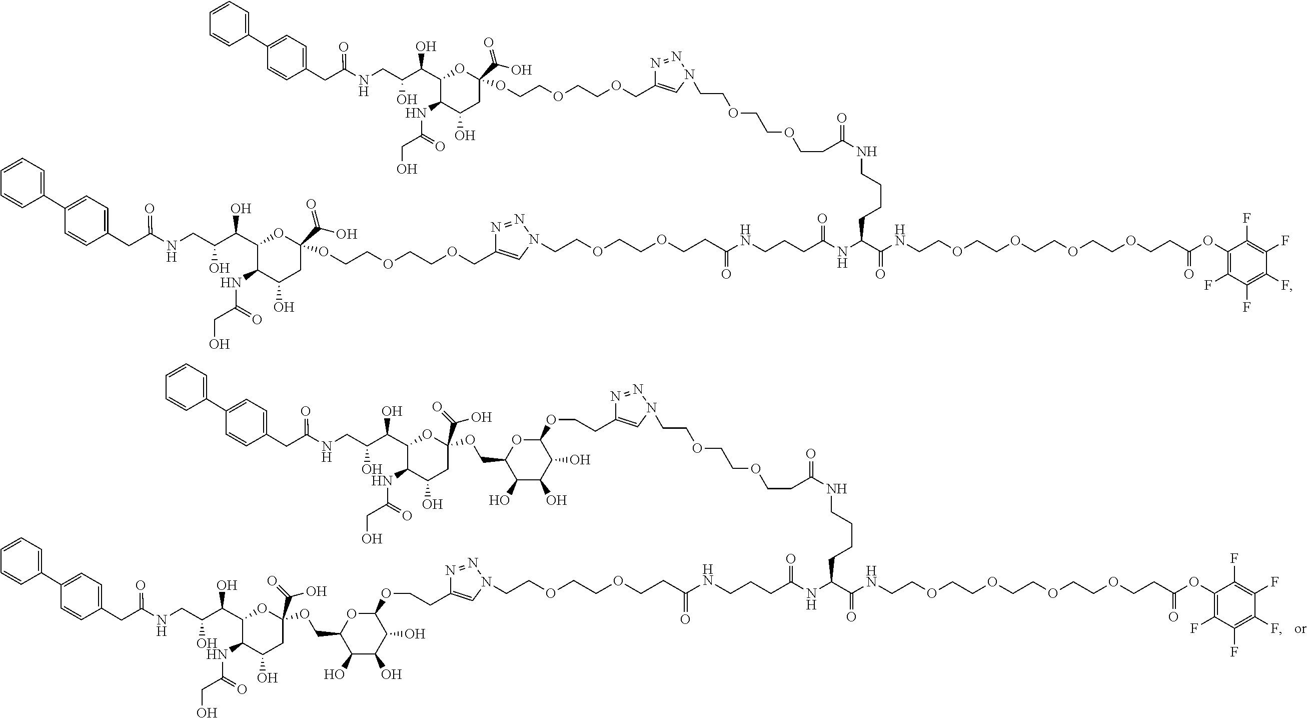

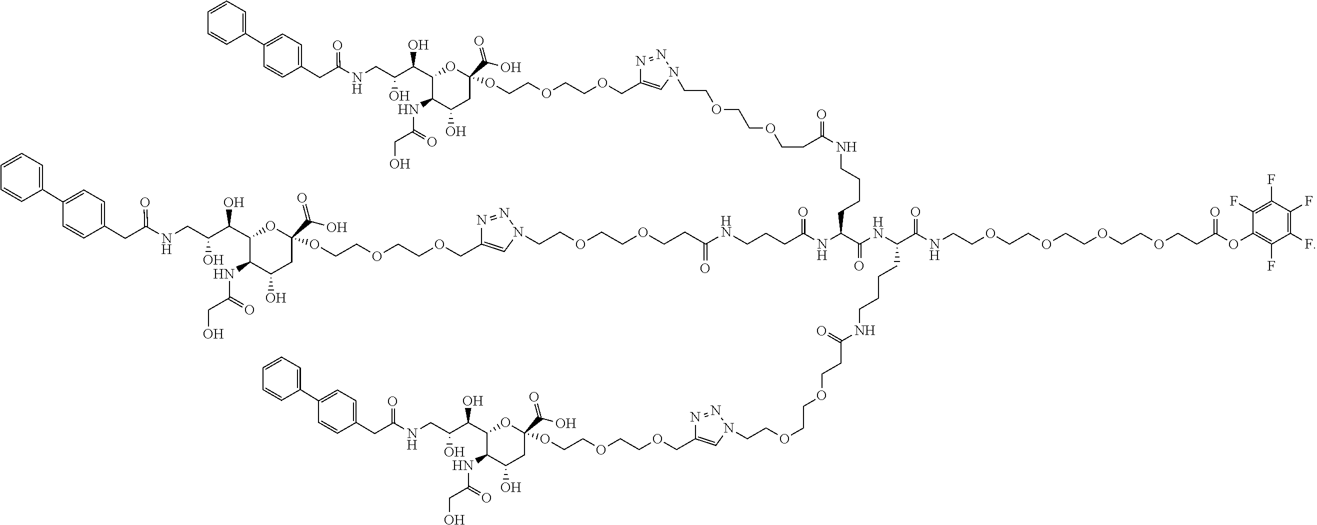 [SMILES: O=C(CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCCC[C@H](NC(=O)CCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)N[C@@H](CCCCNC(=O)CCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)C(=O)NCCOCCOCCOCCOCCC(=O)Oc1c(F)c(F)c(F)c(F)c1F]

132. The engineered autoantigen of claim 121, wherein the autoantigen comprises a polypeptide or a nucleic acid.

133. The engineered autoantigen of claim 132, wherein the autoantigen is a polypeptide and the polypeptide is a PLA2R (Phospholipase A2 Receptor), a Desmoglein (Dsg) DSG-3 or DSG-1, a Gliadin, a type 2 transglutaminase, PDC-E2, Thyroid-stimulating hormone receptor (TSHR), acetylcholine receptor (AChR), muscle-specific tyrosine kinase (MuSK), or LRP4.

134. A pharmaceutical composition, comprising:
the engineered autoantigen of claim 121; and
a pharmaceutical excipient.

135. A method of treating an individual having an autoimmune disease comprising an immune response to an antigen, the method comprising administering to the individual the pharmaceutical composition of claim 134 in an amount effective to treat the autoimmune disorder or disease, wherein the administering results in reduction of antibodies against the antigen in the individual.

136. The method of claim 135, wherein the autoimmune disease or disorder is selected from the group consisting of Pemphigus vulgaris, celiac disease, primary biliary cholangitis, membranous nephropathy, rheumatoid arthritis, Systemic lupus erythematosus, rheumatoid arthritis, psoriatic arthritis, ankylosing spondylitis, Crohn's disease, ulcerative colitis, psoriasis, hidradenitis suppurativa, uveitis, myasthenia gravis, Graves' disease, and juvenile idiopathic arthritis.

137. The method of claim 136, wherein
the autoimmune disorder is Pemphigus vulgaris and the autoantigen is Desmoglein (Dsg) 1 and/or Dsg 3,
the autoimmune disorder is celiac disease and the autoantigen is Gliadin and/or type 2 transglutaminase,
the autoimmune disorder is primary biliary cholangitis and the autoantigen is Pyruvate dehydrogenase complex component E2 (PDC-E2),
the autoimmune disorder is membranous neuropathy and the autoantigen is PLA2R (Phospholipase A2 Receptor),
the autoimmune disorder is myasthenia gravis and the autoantigen is muscle-specific kinase (MuSK), low-density lipoprotein receptor-related protein 4 (LRP4), and/or Acetylcholine Receptor (AChR),
the autoimmune disorder is Graves' disease and the autoantigen is thyroid-stimulating hormone receptor (TSHR),
the autoimmune disorder is rheumatoid arthritis and the autoantigen is citrullinated autoantigen, carbamoylated autoantigen, and/or acetylated autoantigen, or the autoimmune disorder is Systemic lupus erythematosus and the autoantigen is dsDNA, nucleosomes, ribonucleoprotein, and/or Sm antigen.

138. The method of claim 135, wherein the autoantigen is administered to an individual for 1 month or more; 3 months or more; 6 months or more; or 1 year or more.

139. The method of claim 135, wherein the administration to the individual is weekly;
biweekly; monthly; quarterly or semi-annually; annually or bi-annually.

140. An engineered autoantigen comprising an autoantigen fused to a Siglec binding polypeptide or peptide.

141. The engineered autoantigen of claim 140, wherein the Siglec binding polypeptide comprises an antibody that binds to Siglec.

142. A nucleic acid encoding a hypoimmunogenic biotherapeutic, wherein the hypoimmunogenic biotherapeutic is a fusion protein comprising a therapeutically active polypeptide and a siglec binding protein or peptide, wherein the siglec binding protein or peptide binds to a B-cell associated siglec and wherein binding of the siglec binding protein to the siglec suppresses B-cell activation.
